(19) United States
(12) Patent Application Publication
(10) Pub. No.: US 2006/0275283 A1
(43) Pub. Date: Dec. 7, 2006

(54) Fcgamma receptor-binding polypeptide variants and methods related thereto

(75) Inventors: Herman van Vlijmen, Mechelen (BE); Frederick R. Taylor, Milton, MA (US); Ellen Garber, Cambridge, MA (US)

Correspondence Address: LAHIVE & COCKFIELD, 28 STATE STREET, BOSTON, MA, 02109, US

(73) Assignee: Biogen Idec MA Inc., Cambridge, MA (US)

(21) Appl. No.: 11/433,313

(22) Filed: May 11, 2006

Related U.S. Application Data
(63) Continuation of application No. PCT/US04/37948, filed on Nov. 12, 2004.
(60) Provisional application No. 60/519,735, filed on Nov. 12, 2003.
(60) Provisional application No. 60/519,747, filed on Nov. 12, 2003.
(60) Provisional application No. 60/519,734, filed on Nov. 12, 2003.
(60) Provisional application No. 60/519,746, filed on Nov. 12, 2003.

(51) Int. Cl. A61K 39/395; C07K 16/18; G06F 19/00; C07H 21/04; C12P 21/06; C12N 5/06

(52) U.S. Cl. 424/130.1; 435/69.1; 435/320.1; 435/326; 530/387.1; 536/23.53; 702/19

(57) ABSTRACT

The compositions and methods of the present invention are based, in part, on our discovery that an effector function mediated by an Fc-containing polypeptide can be altered by modifying one or more amino acid residues within the polypeptide (by, for example, electrostatic optimization). The polypeptides that can be generated according to the methods of the invention are highly variable, and they can include antibodies and fusion proteins that contain an Fc region or a biologically active portion thereof.

### BACKGROUND OF THE INVENTION

Many biological processes are mediated by the specific interaction of one protein with another. For example, enzymes are proteins that specifically bind their substrates, and substantial information is transmitted from cell to cell when ligands (such as neurotransmitters and hormones) bind their cognate receptors. Among the most fascinating interactions are those that occur in the context of an immune response in which antibodies (also known as immunoglobulins) are produced to defend the body against foreign substances that can cause infection or disease.

Antibodies contain distinct domains that specifically interact with antigens and with receptors on “effector” cells, such as phagocytes. For example, the Fc region mediates effector functions that have been divided into two categories. In the first are the functions that occur independently of antigen binding; these functions confer persistence in the circulation and the ability to be transferred across cellular barriers by transcytosis (see Ward and Ghetie, Therapeutic Immunology 2:77-94, 1995). In the second are the functions that operate after an antibody binds an antigen; these functions involve the participation of the complement cascade or Fc receptor (FcR)-bearing cells.

FcRs are defined by their specificity for immunoglobulin isotypes. For example, Fc receptors for IgG antibodies are referred to as FcγR. FcRs are specialized cell surface receptors on hematopoietic cells that mediate both the removal of antibody-coated pathogens by phagocytosis of immune complexes, and the lysis of erythrocytes and various other cellular targets (e.g. tumor cells) coated with the corresponding antibody. Lysis occurs via antibody dependent cell mediated cytotoxicity (ADCC; see Van de Winkel and Anderson, J Leuk. Biol. 49:511-24, 1991).

Certain Fc receptors, the Fc gamma receptors (FcγRs), play a critical role in either abrogating or enhancing immune recruitment. FcγRs are expressed on leukocytes and are composed of three distinct classes: FcγRI, FcγRII, and FcγRIII. (Gessner et al., Ann. Hematol., (1998), 76: 231-48). Structurally, the FcγRs are all members of the immunoglobulin superfamily, having an IgG-binding α-chain with an extracellular portion composed of either two or three Ig-like domains. Human FcγRI (CD64) is expressed on human monocytes, exhibits high affinity binding (Ka=10⁸-10⁹ M⁻¹) to monomeric IgG1, IgG3, and IgG4. Human FcγRII (CD32) and FcγRII (CD16) have low affinity for IgG1 and IgG3 (Ka<10⁷ M⁻¹), and can bind only complexed or polymeric forms of these IgG isotypes. Furthermore, the FcγRII and FcγTIII classes comprise both “A” and “B” forms.

Mice have the equivalent of FcγRI, FcγRIIb and FcγRIIIa, refered to as FcγRI, II and III. FcγRI and FcγRIIIa are bound by a transmembrane domain and also through association with gamma chain. FcγRIIa and FcγRIIb also have transmembrane domains, but do not associate with gamma chain. FcγRIIIb is the only receptor that associated with cell memebranes via a phosphatidyl inositol glycan (GPI). Human FcγRIIIa, is the only receptor found on NK cells and there is genetic proof of its involvement in ADCC in vivo.

Binding of the Fc portion of an antibody to an Fc receptor causes certain immune effects, for example, endocytosis of immune complexes, engulfment and destruction of antibody-coated particles or microorganisms (also called antibody-dependent phagocytosis, or ADCP), clearance of immune complexes, lysis of antibody-coated target cells by killer cells (called antibody-dependent cell-mediated cytotoxicity, or ADCC), release of inflammatory mediators, regulation of immune system cell activation, and regulation of antibody production.

Monoclonal antibodies (mAbs) have now been used to treat disease in human patients (King and Adair, Curr. Opin. Drug Discovery Dev. 2:110-117, 1999; Vaswani and Hamilton, Ann. Allergy Asthma Immunol. 81:105-119, 1998; and Hollinger and Hoogenboom, Nature Biotechnol. 16:1015-1016, 1998). Although some mAbs may function effectively without utilizing antibody effector functions (e.g. neutralizing antibodies), in many cases it may be desirable to engineer the Fc portion of the antibody to recruit the immune system to elicit an immune response.

In clinical applications where destruction of a target cell is desired, antigen-dependent effector responses may be required for therapeutic antibodies to be effective. For example, antigen-dependent effector responses are necessary to eliminate tumor cells or to deplete the immune cells involved in inflammation and autoimmunity. Antibodies provided as cancer or autoimmune therapeutics should, therefore, evoke these antigen-dependent effector functions.

Alternatively, antibody therapeutics with reduced or eliminated effector function may be desired, e.g., in situations where activation of effector function may provoke unwanted side effects. One example of an effector-mediated side effect is the release of inflammatory cytokines causing an acute fever reaction. In addition, depletion of certain cell populations may be undesirable. For example, in the case of therapeutic antibodies (e.g. anti-inflammatory blocking antibodies) whose mechanism of action invoves blocking or antagonism but not killing of the cells bearing the target antigen, e.g. T cells.

The effector function of an antibody can be avoided by using antibody fragments lacking the Fc region (e.g., such as a Fab, Fab′2, or single chain antibody (sFv)) however these fragments have a reduced half-life, only one antigen binding site instead of two (e.g., in the case of Fab antibody fragments and single chain antibodies (sFv)), and are more difficult to purify. Accordingly, there is a need for antibodies (and other Fc-containing polypeptides such as fusion proteins) where the antigen-independent effector finctions are tailored for the intended use of the antibody. Similarly, there is a need for methods that would allow for prediction of changes in antibody sequence which will alter the antigen-independent effector functions (thus obviating the need to rely on laborious trial-and-error processes). Such therapeutics and methods or making them would be of great benefit.

### SUMMARY OF THE INVENTION

The present invention features altered polypeptides having specific amino acid substitutions within, for example, an Fc region or an FcR binding fragment thereof (e.g. polypeptides having amino acid substitutions within an IgG constant domain), that confer alterations in antigen-independent effector function (e.g. ADCC or complement activation). Methods for producing the altered polypeptides and utilizing them as protein-based therapeutics are also provided.

The present invention is based, at least in part, on the identification of particular amino acid residues within the constant domain (Fc) of human Fc region (specifically, Fc region derived from the IgG antibodies) that, when altered by one or more amino acid mutation, alter the antigen-dependent effector functions of the antibody. Accordingly, the invention features polypeptides, e.g., antibodies and fusion proteins that contain all or part of an Fc region, that have been mutated at one or more amino acid residues to increase or decrease the antigen-dependent effector functions of the polypeptide.

The instant invention further provides techniques for identifying desirable amino acid mutations and methods for producing the polypeptides comprising such mutations. The methods include molecular modeling, which can be used to predict amino acid alterations in an amino acid sequence to alter (e.g., enhance or reduce) binding to an Fc receptor, e.g. a human Fcγ receptor. Generally, the methods begin with a “starting” or “target” polypeptide, or a complex (e.g. crystal strucuture or homology model) containing the first polypeptide bound to FcR, and modification of the first polypeptide results in a “second” or “altered” polypeptide, which differs from the first polypeptide in a way that allows the altered polypeptide to perform better in a particulartherapeutic or diagnostic application. For example, the second polypeptide may more efficiently carry out one or more antigen-dependent effector functions (e.g. ADCC or complement activation). The modeling can be carried out in silico. In one aspect, the invention pertains to an altered polypeptide comprising at least an FcγR binding portion of an Fc region wherein the polypeptide comprises at least one mutation compared to a starting polypeptide and wherein the at least one mutation is selected from the group consisting of:

a substitution at EU amino acid position 236;

a substitution at EU amino acid position 239 with proline;

a substitution at EU amino acid position 241 with glutamine or histidine;

a substitution at EU amino acid position 251 with a non-polar amino acid or serine;

a substitution at EU amino acid position 265 with a negatively charged amino acid;

a substitution at EU amino acid position 268 with proline or a negatively charged amino acid;

a substitution at EU amino acid position 294 with serine, threonine, or asparagine;

a substitution at EU amino acid position 301 with serine, threonine, asparagine, glutamine or a charged amino acid;

a substitution at EU amino acid position 328 with lysine;

a substitution at EU amino acid position 332 with lysine;

a substitution at EU amino acid position 376 with a polar amino acid or a charged amino acid;

a substitution at EU amino acid position 378 with a charged amino acid, phenylalanine, glutamine, arginine, tyrosine, or tryptophan;

a substitution at EU amino acid position 388; and

a substitution at EU amino acid position 435 with a polar amino acid or glycine.

In another aspect, the invention pertains to an altered polypeptide comprising at least an FcγR binding portion of an Fc region wherein the polypeptide comprises at least one mutation compared to a starting polypeptide and wherein the at least one mutation is selected from the group consisting of:

a substitution of glycine at EU amino acid position 236;

a substitution of serine at EU amino acid position 239 with proline;

a substitution of phenylalanine at EU amino acid position 241 with glutamine or histidine;

a substitution of leucine at EU amino acid position 251 with a non-polar amino acid or serine;

a substitution of aspartate at EU amino acid position 265 with a negatively charged amino acid;

a substitution of histidine at EU amino acid position 268 with proline or a negatively charged amino acid;

a substitution of glutamine or glutamate at EU amino acid position 294 with serine, threonine, or asparagine;

a substitution of arginine at EU amino acid position 301 with serine, threonine, asparagine, glutamine or a charged amino acid;

a substitution of leucine at EU amino acid position 328 with lysine;

a substitution of isoleucine at EU amino acid position 332 with lysine;

a substitution of asparagine at EU amino acid position 376 with a polar amino acid or a charged amino acid;

a substitution of alanine at EU amino acid position 378 with a charged amino acid, phenylalanine, glutamine, arginine, tyrosine, or tryptophan;

a substitution of glutamate at EU amino acid position 388; and

a substitution of histidine at EU amino acid position 435 with a polar amino acid or glycine.

In one embodiment, the amino acid at any of EU amino acid positions 236 or 388 is replaced with a non-polar amino acid, a charged amino acid, or a polar amino acid.

In another embodiment, the charged amino acid is a negatively charged amino acid.

In one embodiment, the negatively charged amino acid is selected from the group consisting of aspartate and glutamate.

In another embodiment, the charged amino acid is a positively charged amino acid.

In yet another embodiment, the positively charged amino acid is selected from the group consisting of arginine, histidine, and lysine.

In one embodiment, the polar amino acid is selected from the group consisting of methionine, phenylalanine, tryptophan, serine, tyrosine, asparagine, glutamine, and cysteine.

In one embodiment, the non-polar amino acid is selected from the group consisting of alanine, leucine, isoleucine, valine, glycine, and proline.

In one embodiment, a polypeptide further comprises a mutation selected from the group consisting of:

a substitution at EU amino acid position 234 with aspartate or glutamine;

a substitution at EU amino acid position 239 with aspartate, glutamate, or histidine;

a substitution at EU amino acid position 270 with glutamate;

a substitution at EU amino acid position 292 with alanine;

a substitution at EU amino acid position 293 with aspartate;

a substitution at EU amino acid position 294 with alanine or asparagine;

a substitution at EU amino acid position 296 with alanine, serine, asparagine, glutamine, threonine, histidine, or phenylalanine;

a substitution at EU amino acid position 298 with alanine or asparagine;

a substitution at EU amino acid position 301 with alanine;

a substitution at EU amino acid position 326 with aspartate, glutamate, asparagine, or glutamine;

a substitution at EU amino acid position 328 with asparagine, aspartate, glutamate, glutamine, or threonine;

a substitution at EU amino acid position 330 with histidine or leucine;

a substitution at EU amino acid position 332 with aspartate, glutamate, glutamine, or histidine;

a substitution at EU amino acid position 333 with aspartate;

a substitution at EU amino acid position 334 with asparagine, aspartate, glutamine, glutamate, valine, or arginine; and

a substitution at EU amino acid position 338 with methionine.

In another aspect, the invention pertains to an altered polypeptide comprising at least an FcγR binding portion of an Fc region wherein the polypeptide comprises at least two mutations compared to a starting polypeptide and wherein the at least two mutations are selected from the group consisting of:

a substitution at EU position 239 with glutamate or asparate and a substitution of EU position 378 with phenylalanine, tryptophan, tyrosine, glycine, or serine;

a substitution at EU position 332 with aspartate and a substitution of EU position 378 with phenylalanine, lysine, tryptophan, or tyrosine;

a substitution at EU position 332 with aspartate and a substitution of EU position 435 with glycine or serine; and

a substitution at EU position 332 with aspartate and a substitution of EU position 261 with alanine.

In one embodiment, the altered polypeptide is an antibody or fragment thereof.

In another embodiment, the altered polypeptide is a fusion protein.

In one embodiment, the FcγR binding portion or the Fc region is derived from a human antibody.

In another embodiment, the FcγR binding portion comprises a complete Fc region.

In one embodiment, the starting polypeptide comprises the amino acid sequence of SEQ ID NO. 2.

In another embodiment, the antibody is of the IgG isotype.

In another embodiment, the IgG isotype is of the IgG1 subclass.

In one embodiment, the polypeptide comprises one or more non-human amino acids residues in a complementarity determining region (CDR) of V_{L} or V_{H}.

In one embodiment, the polypeptide binds (a) an antigen and (b) an FcR.

In another embodiment, the antigen is a tumor-associated antigen.

In one embodiment, the polypeptide binds (a) a ligand and (b) an FcR.

In one embodiment, the FcR is an FcγR.

In another embodiment, the polypeptide binds the FcR with different binding affinity than the starting polypeptide that does not contain the mutation.

In yet another embodiment. the binding affinity of the altered polypeptide is about 1.5-fold to about 100-fold greater.

In another embodiment, the binding affinity of the altered polypeptide is about 1.5-fold to about 100-fold lower.

In one embodiment, the altered polypeptide, when administered to a patient, exhibits an antigen-dependent effector function that is different from the starting polypeptide that does not contain the mutation.

In one embodiment, the altered polypeptide binds to Protein A or G.

In another aspect, the invention pertains to a pharmaceutical composition comprising the altered polypeptide of claim 1 or 2.

In another embodiment, the invention pertains to a nucleic acid molecule comprising a sequence encoding the polypeptide of of the invention.

In one embodiment, the nucleic acid molecule is in an expression vector. In one embodiment, the invention pertains to a host cell comprising the expression vector of claim 31.

In another aspect, the invention pertains to a method for treating a patient suffering from a disorder, the method comprising administering to the patient an altered polypeptide comprising at least an FcγR binding portion of an Fc region which comprises at least one mutation selected from the group consisting of:

a substitution of leucine at EU amino acid position 251 with alanine or glycine;

a substitution of histidine at EU amino acid position 268 with aspartate;

a substitution of alanine at EU amino acid position 330 with leucine or histidine;

a substitution of isoleucine at EU amino acid position 332 with aspartate, glutamate, or glutamine;

a substitution of lysine at EU amino acid position 334 with arginine;

a substitution of alanine at EU amino acid position 378 with phenylalanine, lysine, tryptophan, or tyrosine; and

a substitution of histidine at EU amino acid position 435 with glycine or serine wherein the altered polypeptide exhibits an antigen-dependent effector function that is enhanced relative to the starting polypeptide that does not contain the mutation.

In one embodiment, the altered polypeptide further comprises of a serine at EU amino acid position 239 with aspartate or glutamate.

In another embodiment, the altered polypeptide comprises two mutations, wherein the two mutations are selected from the group consisting of: S239E/1332D, S239E/I332E, S239D/I332D, S239D/I332E, S239D/A378F, S239D/A378K, S239D/A378F, S239D/A378W, S239D/A378Y, S239D/A378G, S239D/A378S, 1332D/A378F, 1332D/A378W, or I332D/A378Y.

In another aspect, the invention pertains to a method for treating a patient suffering from a disorder, the method comprising administering to the patient an an altered polypeptide comprising at least an FcγR binding portion of an Fc region which comprises at least one mutation selected from the group consisting of:

a substitution of glycine at EU amino acid position 236 with alanine;

a substitution of serine at EU amino acid position 239 with proline;

a substitution of phenylalanine at EU amino acid position 241 with glutamine or histidine;

a substitution of leucine at EU amino acid position 251 with glycine;

a substitution of leucine at EU amino acid position 261 with alanine;

a substitution of aspartate at EU amino acid position 265 with glutamate;

a substitution of leucine at EU amino acid position 268 with proline;

a substitution of glutamate at EU amino acid position 293 with aspartate;

a substitution of glutamate at EU amino acid position 294 with serine or threonine;

a substitution of arginine at EU amino acid position 301 with lysine, asparagine, glutamine, serine, or threonine;

a substitution of leucine at EU amino acid position 328 with glutamine, aspartate, lysine, or threonine;

a substitution of isoleucine at EU amino acid position 332 with lysine;

a substitution of asparagine at EU amino acid position 376 with arginine, lysine, histidine, phenylalanine, or tryptophan;

a substitution of alanine at EU amino acid position 378 with histidine; and

a substitution of histidine at EU amino acid position 435 with alanine, serine, or glycine

wherein the altered polypeptide exhibits an antigen-dependent effector function that is reduced relative to the starting polypeptide that does not contain the mutation.

In yet another aspect, the invention pertains to a method of producing the altered polypeptide of claim 1 or 2, the method comprising:

(a) transfecting a cell with the nucleic acid molecule comprising a nucleotide sequence that encodes the altered polypeptide; and

(b) purifying the altered polypeptide from the cell or cell supernatant.

In yet another aspect, the invention pertains to a method of producing the antibody of claim 16 or 17, the method comprising:

(a) providing a first nucleic acid molecule comprising a nucleotide sequence that encodes the variable (V_{L}) and constant regions (C_{L}) of the antibody's light chain;

(b) providing a second nucleic acid molecule comprising a nucleotide sequence that encodes the variable (V_{H}) and constant regions (CH₁, CH₂, and CH₃) of the antibody's heavy chain;

(c) transfecting a cell with the first and second nucleic acid molecules under conditions that permit expression of the altered antibody comprising the encoded light and heavy chains; and

(d) purifying the antibody from the cell or cell supernatant.

In one embodiment, the cell is a 293 cell.

In yet another aspect, the invention pertains to a method for identifying a polypeptide with an altered binding affinity for a FcγR compared to a starting polypeptide, the method comprising:

(a) determining a spatial representation of an optimal charge distribution of the amino acids of the starting polypeptide and an associated change in binding free energy of the starting polypeptide when bound to the FcγR in a solvent;

(b) identifying at least one candidate amino acid residue position of the starting polypeptide to be modified to alter the binding free energy of the starting polypeptide when bound to the FcγR; and

(c) identifying an elected amino acid at the amino acid position, such that substitution of the elected amino acid into the starting polypeptide results in an altered polypeptide with an altered binding affinity for the FcγR.

In one embodiment, the method further comprises incorporating the elected amino acid in the starting polypeptide to form an altered polypeptide.

In another embodiment, the method further comprises calculating the change in the free energy of binding of the altered Fc-containing polypeptide when bound to the FcγR, as compared to the starting polypeptide when bound to the FcγR.

In another embodiment, the calculating step first comprises modeling the mutation in the starting polypeptide in silico, and then calculating the change in free energy of binding.

In one embodiment, the calculating step uses at least one determination selected from the group consisting of a determination of the electrostatic binding energy using a method based on the Poisson-Boltzmann equation, a determination of the van der Waals binding energy, and a determination of the binding energy using a method based on solvent accessible surface area.

In one embodiment, the amino acid substitution results in incorporation of an elected amino acid with a different charge than the candidate amino acid.

In another embodiment, an elected amino acid with a different solvation effect than the candidate amino acid. the amino acid substitution results in incorporation of an elected amino acid with a different dielectric constant than the candidate amino acid.

In one embodiment, the substitution increases the free energy of binding between altered Fc-containing polypeptide and FcγR when bound in a solvent, thereby decreasing binding affinity of the altered Fc-containing polypeptide for FcγR.

In another embodiment, the substitution decreases the free energy of binding between altered Fc-containing polypeptide and FcγR when bound in a solvent, thereby increasing binding affinity of the altered Fc-containing polypeptide for FcγR.

In yet another aspect, the invention pertains to an altered polypeptide comprising at least one amino acid mutation not found in a starting polypeptide, wherein the altered polypeptide exhibits a different binding affinity for an FcR as compared to the starting polypeptide, and wherein the altered polypeptide comprises an amino acid sequence predicted by the method of claim 40.

In another aspect, the invention pertains to a pharmaceutical composition comprising a polypeptide of the invention.

In another embodiment, the invention pertains to a nucleic acid molecule comprising a nucleotide sequence encoding a polypeptide of the invention.

In one embodiment, the polypeptide exhibits at least one altered antigen dependent effector function selected from the group consisting of: opsonization, phagocytosis, complement dependent cytotoxicity, antigen-dependent cellular cytotoxicity (ADCC), or effector cell modulation.

In one embodiment, the FcγR is an activating FcγR.

In one embodiment, the activating FcγR is an FcγRI, FcγRIIa, or FcγRIIIa.

In another embodiment, the FcγR is an inhibitory FcγR.

In another embodiment, the inhibitory FcγR is FcγRIIb.

The details of one or more embodiments of the invention are set forth in the accompanying drawings and the description below. Other features, objects, and advantages of the invention will be aparent from the description and drawings, and from the claims. The contents of any patents, patent applications, and other references cited in our specification are hereby incorporated by reference in their entirety.

### BRIEF DESCRIPTION OF THE FIGURES

FIG. 1A shows the DNA sequence of a mature murine/human chimeric heavy chain of the chimeric antibody chCB6-huIgG1, which was utilized as a starting polypeptide in the methods of the invention. FIG. 1B shows the predicted amino acid sequence of the mature chCB6-huIgG1 heavy chain.

FIG. 2 shows the amino acid sequence of the Fc region of the chCB6-huIgG1 heavy chain used as a starting polypeptide in the methods of the invention. Amino acid positions are indicated by EU numbering.

FIG. 3A shows the DNA sequence of the kappa light chain of the chCB6-huIgG1 chimeric antibody. FIG. 3B shows the amino acid sequence of the chCB6-huIgG1 kappa light chain.

FIGS. 4A, B, and C show the results obtained using cell-based bridging assays for evaluation of the FcγR binding affinity of select altered antibodies of the invention in comparison with the starting (wild-type) antibody chCB6-huIgG1. FIG. 4A illustrates results obtained in with altered antibodies containing mutations at EU positions 328 and 332 (L328N, I332H, I332E) in a bridging assay with a human FcγRIII (CD16). FIG. 4B illustrates results obtained in with altered antibodies containing mutations at EU positions 299 and 334 (T299C, K334Q, K334V) in a bridging assay with human FcγRIIb (CD32b). FIG. 4C illustrates results obtained in with altered antibodies containing mutations at EU positions 299 and 334 (T299C, K334V, and the triple mutant S298A/E333A/K334A as described by Shields et al (JBC 276, 6591-6604 (2001) in a bridging assay with a human FcγRI (CD64).

FIG. 5 shows the results obtained using ELISA binding assay for evaluation of the C1q binding affinity of select altered antibodies (containing the mutations D376W and H435G) of the invention in comparison with the starting (wild-type or “WTCB6”) antibody chCB6-huIgG1.

FIG. 6 shows the results obtained using an AlphaScreen assay for evaluation of the relative FcγRIII (CD 16) binding affinity of select altered polypeptides (those containing mutations I332E, I332D, S239D, S239E, T299C, and the triple mutant S298A/E333A/K334A) of the invention in comparison with the starting (wild-type or) “WTCB6”) antibody chCB6-huIgG1

FIG. 7 shows the results obtained using a T cell and NK cell cytolysis assay for evaluation of the relative antibody-dependent cell-mediated cytotoxicity (ADCC) effectors functions of select altered antibodies (those containing mutations I332E, T299C, and the triple mutant S298A/E333A/K334A) of the invention in comparison with the starting (wild-type or “CB6”) antibody chCB6-huIgG1.

### DETAILED DESCRIPTION

The instant invention is based, at least in part, on the identification of polypeptides (such as antibodies and fusion proteins) that include at least a portion of a Fc region (e.g., a constant domain of an immunoglobulin such as IgG1) which exhibit altered binding to an Fc receptor (e.g., CD16). Such altered polypeptides exhibit either increased or decreased binding to FcR when compared to wild-type polypeptides and, therefore, mediate enhanced or reduced effector function, respectively. Fc region variants with improved affinity for FcR are anticipated to enhance effector finction, and such molecules have useful applications in methods of treating mammals where target molecule destruction is desired, e.g., in tumor therapy. In contrast, Fc region variants with decreased FcR binding affinity are expected to reduce effector function, and such molecules are also useful, for example, for treatment of conditions in which target cell destruction is undesirable, e.g., where normal cells may express target molecules, or where chronic administration of the polypeptide might result in unwanted immune system activation.

The invention also pertains to methods of making such altered polypeptides and to methods of using such polypeptides.

Various aspects of the invention are described in further detail in the following subsections:

I. Definitions

The terms “protein,” “polypeptide,” and “peptide” are used interchangeably herein. A protein may comprise one or more of the natural amino acids or non-natural amino acids.

A “starting polypeptide” or “first polypeptide” is a polypeptide comprising an amino acid sequence which lacks one or more of the Fc region modifications disclosed herein and which differs in effector function compared to an altered or modified polypeptide. A starting polypeptide is a naturally occurring or artificially-derived polypeptide containing an Fc region, or FcR binding portion thereof. The starting polypeptide may comprise a naturally occurring Fc region sequence or an Fc region with pre-existing amino acid sequence modifications (such as additions, deletions and/or substitutions). The starting polypeptides of the invention are modified as disclosed herein to to modulate (either to increase or decrease) binding affinity toFcR.

As used herein, the term “altered polypeptide” or “second polypeptide” refers to a polypeptide comprising a non-naturally occurring Fc binding portion which comprises at least one mutation in the Fc region. When we say that an altered polypeptide exhibits an “altered effector function”, we mean that the altered polypeptide facilitates one or more (and possibily, but not necessarily, all) of its effector functions to a greater or lesser extent than the starting polypeptide.

As used herein, the term “Fc region” includes amino acid sequences derived from the constant region of an antibody heavy chain. The Fc region is the portion of a heavy chain constant region of an antibody beginning N-terminal of the hinge region at the papain cleavage site, at about position 216 according to the EU index and including the hinge, CH2, and CH3 domains.

The starting polypeptide can comprise at least a portion of an Fc region that mediates binding to FcR. For example, in one embodiment, a starting polypeptide is an antibody or an Fc fusion protein. As used herein, the term “fusion protein” refers to a chimeric polypeptide which comprises a first amino acid sequence linked to a second amino acid sequence with which it is not naturally linked in nature. For example, a fusion protein may comprise an amino acid sequence encoding least a portion of an Fc region (e.g., the portion of the Fc region that confers binding to FcR) and an amino acid sequence encoding a non-immunoglobulin polypeptide, e.g., a ligand binding domain of a receptor or a receptor binding domain of a ligand. The amino acid sequences may normally exist in separate proteins that are brought together in the fusion polypeptide or they may normally exist in the same protein but are placed in a new arrangement in the fusion polypeptide. A fusion protein may be created, for example, by chemical synthesis, or by creating and translating a polynucleotide in which the peptide regions are encoded in the desired relationship.

As used herein, the terms “linked,” “fused” or “fusion” are used interchangeably. These terms refer to the joining together of two more elements or components, by whatever means including chemical conjugation or recombinant means. An “in-frame fusion” or “operably linked” refers to the joining of two or more open reading frames (ORFs) to form a continuous longer ORF, in a manner that maintains the correct reading frame of the original ORFs. Thus, the resulting recombinant fusion protein is a single protein containing two ore more segments that correspond to polypeptides encoded by the original ORFs (which segments are not normally so joined in nature.) Although the reading frame is thus made continuous throughout the fused segments, the segments may be physically or spatially separated by, for example, an in-frame linker sequence.

In one embodiment, a polypeptide of the invention comprises an immunoglobulin antigen binding site or the portion of a receptor molecule responsible for ligand binding or the portion of a ligand molecule that is responsible for receptor binding.

As used herein, the term “effector function” refers to the functional ability of the Fc region or portion thereof to bind proteins and/or cells of the immune system and mediate various biological effects. Effector functions may be antigen-dependent or antigen-independent.

As used herein, the term “antigen-dependent effector function” refers to an effector function which is normally induced following the binding of an antibody to a corresponding antigen. Typical antigen-dependent effector functions include the ability to bind a complement protein (e.g. C1q). For example, binding of the C1 component of complement to the Fc region can activate the classical complement system leading to the opsonisation and lysis of cell pathogens, a process referred to as complement-dependent cytotoxicity (CDCC). The activation of complement also stimulates the inflammatory response and may also be involved in autoimmune hypersensitivity.

Other antigen-dependent effector functions are mediated by the binding of antibodies, via their Fc region, to certain Fc receptors (“FcRs”) on cells. There are a number of Fc receptors which are specific for different classes of antibody, including IgG (gamma receptors, or IgγRs), IgE (epsilon receptors, or IgεRs), IgA (alpha receptors, or IgαRs) and IgM (mu receptors, or IgμRs). Binding of antibody to Fc receptors on cell surfaces triggers a number of important and diverse biological responses including endocytosis of immune complexes, engulfment and destruction of antibody-coated particles or microorganisms (also called antibody-dependent phagocytosis, or ADCP), clearance of immune complexes, lysis of antibody-coated target cells by killer cells (called antibody-dependent cell-mediated cytotoxicity, or ADCC), release of inflammatory mediators, regulation of immune system cell activation,placental transfer and control of immunoglobulin production.

Certain Fc receptors, the Fc gamma receptors (FcγRs), play a critical role in either abrogating or enhancing immune recruitment. FcγRs are expressed on leukocytes and are composed of three distinct classes: FcγRI, FcγRII, and FcγRIII. the Fc region of the IgG immunoglobulin isotype (Gessner et al., Ann. Hematol., (1998), 76: 231-48). Structurally, the FcγRs are all members of the immunoglobulin superfamily, having an IgG-binding α-chain with an extracellular portion composed of either two or three Ig-like domains. Human FcγRI (CD64) is expressed on human monocytes, exhibits high affinity binding (Ka=10⁸-10⁹ M⁻¹) to monomeric IgG1, IgG3, and IgG4. Human FcγRII (CD32) and FcryRII (CD16) have low affinity for IgG1 and IgG3 (Ka<107 M⁻¹), and can bind only complexed or polymeric forms of these IgG isotypes.

As used herein, the term “antigen-independent effector function” refers to an effector function which may be induced by an antibody, regardless of whether it has bound its corresponding antigen. Typical antigen-independent effector functions include cellular transport, circulating half-life and clearance rates of immunoglobulins. A structurally unique Fc receptor, the “neonatal Fc receptor” or “FcRn”, also known as the salvage receptor, plays a critical role in regulating these functions. Preferably an FcR to which a polypeptide of the invention binds is a human FcR.

As used herein, the term “activating Fc receptor” refers to Fc receptors (e.g. FcγRI, FcγRIIa, and FcγRIIIa) that are positive regulators of antigen-dependent effector functions. Typically, these receptors are characterized by the presence of an intracellular domain containing an immunoreceptor tyrosine-based activation motif (ITAM).

As used herein, the term “inhibitory Fc receptor” refers to Fc receptors (e.g. FcγRIIb) that are that are negative regulators of antigen-dependent effector functions. Typically, inhibitory Fc receptors are characterized by the presence of a immunoreceptor tyrosine-based inhibition motif (ITIM).

As used herein, the term “mutation” includes substitutions, additions, or deletions of amino acids made in a starting polypeptide to obtain an alterated polypeptide.

An “amino acid substitution” refers to the replacement of at least one existing amino acid residue in a predetermined amino acid sequence (an amino acid sequence of a starting polypeptide) with another different “replacement” amino acid residue. The replacement residue or residues may be “naturally occurring amino acid residues” (i.e. encoded by the genetic code) and selected from the group consisting of: alanine (A); arginine (R); asparagine (N); aspartic acid (D); cysteine (C); glutamine (Q); glutamic acid (E); glycine (G); histidine (H); Isoleucine (I): leucine (L); lysine (K); methionine (M); phenylalanine (F); proline (P): serine (S); threonine (T); tryptophan (W); tyrosine (Y); and valine (V). Substitution with one or more non-naturally occurring amino acid residues is also encompassed by the definition of an amino acid substitution herein. A “non-naturally occurring amino acid residue” refers to a residue, other than those naturally occurring amino acid residues listed above, which is able to covalently bind adjacent amino acid residues(s) in a polypeptide chain. Examples of non-naturally occurring amino acid residues include norleucine, omithine, norvaline, homoserine and other amino acid residue analogues such as those described in Ellman et al. Meth. Enzym. 202:301-336 (1991). To generate such non-naturally occurring amino acid residues, the procedures of, e.g., Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA.

As used herein, the term “non-polar” includes amino acids that have uncharged side chains (e.g. A, L, I, V, G, P). These amino acids are usually implicated in hydrophobic interactions

As used herein, the term “polar” includes amino acids that have net zero charge, but have non-zero partial charges in different portions of their side chains (e.g. M, F, W, S, Y, N, Q, C). These amino acids can participate in hydrophobic interactions and electrostatic interactions.

As used herein, the term “charged” amino acids that can have non-zero net charge on their side chains (e.g. R, K, H, E, D). These amino acids can participate in hydrophobic interactions and electrostatic interactions.

An “amino acid insertion” refers to the incorporation of at least one amino acid into a predetermined amino acid sequence. While the insertion will usually consist of the insertion of one or two amino acid residues, the present larger “peptide insertions”, can be made, e.g. insertion of about three to about five or even up to about ten amino acid residues. The inserted residue(s) may be naturally occurring or non-naturally occurring as disclosed above.

An “amino acid deletion” refers to the removal of at least one amino acid residue from a predetermined amino acid sequence.

As used herein the term “sufficient steric bulk” includes those amino acids having side chains which occupy larger 3 dimensional space. Exemplary amino acid having side chain chemistry of sufficient steric bulk include tyrosine, tryptophan, arginine, lysine, histidine, glutamic acid, glutamine, and methionine, or analogs or mimetics thereof.

As used herein the term “solvent accessible surface area” means the surface area of atoms in contact with solvent molecules. Solvent accessible surface area can be calculated using methods well known in the art. Briefly, an atom or group of atoms is defined as accessible if a solvent (water) molecule of specified size can be brought into van der Waals' contact. van der Waals' contact is the locus of the center of a solvent molecule as it rolls along the protein making the maximum permitted contact.

The term “binding affinity”, as used herein, includes the strength of a binding interaction and therefore includes both the actual binding affinity as well as the apparent binding affinity. The actual binding affinity is a ratio of the association rate over the disassociation rate. Therefore, conferring or optimizing binding affinity includes altering either or both of these components to achieve the desired level of binding affinity. The apparent affinity can include, for example, the avidity of the interaction.

The term “binding free energy” or “free energy of binding”, as used herein, includes its art-recognized meaning, and, in particular, as applied to Fc-Fc recpeptor interactions in a solvent. Reductions in binding free energy enhance affinities, whereas increases in binding free energy reduce affinities.

The term “binding domain” or “binding site” as used herein refers to the one or more regions of the polypeptide that mediate specific binding with a target molecule (e.g. an antigen, ligand, receptor, substrate or inhibitor). Exemplary binding domains include an antibody variable domain, a receptor binding domain of a ligand, a ligand binding domain of a receptor or an enzymatic domain. The term “ligand binding domain” as used herein refers to any native receptor (e.g., cell surface receptor) or any region or derivative thereof retaining at least a qualitative ligand binding ability, and preferably the biological activity of a corresponding native receptor. The term “receptor binding domain” as used herein refers to any native ligand or any region or derivative thereof retaining at least a qualitative receptor binding ability, and preferably the biological activity of a corresponding native ligand. In one embodiment, the polypeptides have at least one binding domain specific for a molecule targeted for reduction or elimination, e.g., a cell surface antigen or a soluble antigen. In preferred embodiments, the binding domain is an antigen binding site.

In a preferred embodiment, the polypeptides of the invention comprise at least one binding site (e.g., antigen binding site, receptor binding site, or ligand binding site). In one embodiment, the polypeptides of the invention comprise at least two binding sites. In one embodiment, the polypeptides comprise three binding sites. In another embodiment, the polypeptides comprise four binding sites.

The polypeptides of the invention may be either monomers or multimers. For example, in one embodiment, the polypeptides of the invention are dimers. In one embodiment, the dimers of the invention are homodimers, comprising two identical monomeric subunits. In another embodiment, the dimers of the invention are heterodimers, comprising two non-identical monomeric subunits. The subunits of the dimer may comprise one or more polypeptide chains. For example, in one embodiment, the dimers comprise at least two polypeptide chains. In one embodiment, the dimers comprise two polypeptide chains. In another embodiment, the dimers comprise four polypeptide chains (e.g., as in the case of antibody molecules).

The term “exposed” amino acid residue, as used herein, includes one in which at least part of its surface is exposed, to some extent, to solvent when present in a polypeptide in solution. Preferably, the exposed amino acid residue is one in which at least about one third of its side chain surface area is exposed to solvent. Various methods are available for determining whether a residue is exposed or not, including an analysis of a molecular model or structure of the polypeptide.

The terms “variant”, “altered polypeptide,” “modified polypeptide”, “polypeptide containing a modified amino acid” and the like, as used herein, include polypeptides which have an amino acid sequence which differs from the amino acid sequence of a starting polypeptide. Typically such polypeptides have one or more mutations, e.g., one or more amino acid residues which have been substituted with another amino acid residue or which has one or more amino acid residue insertions or deletions. Preferably, the polypeptide comprises an amino acid sequence comprising at least a portion of an Fc region which is not naturally occurring. Such variants necessarily have less than 100% sequence identity or similarity with the starting antibody. In a preferred embodiment, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100%, and most preferably from about 95% to less than 100%. In one embodiment, there is one amino acid difference between a starting antibody and a modified antibody of the invention. Identity or similarity with respect to this sequence is defined herein as the percentage of amino acid residues in the candidate sequence that are identical (i.e. same residue) with the starting amino acicd residues, after aligning the sequences and introducing gaps, if necessary, to achieve the maximum percent sequence identity. The modified polypeptides of the present invention may either be expressed, or alternatively, may be modeled in silico.

The phrase “candidate amino acid residue position”, as used herein, includes an amino acid position(s) identified within a polypeptide of the present invention, wherein the substitution of the candidate amino acid is modeled, predicted, or empirically found to modulate FcR binding affinity of the polypeptide upon alteration, deletion, insertion, or substitution with another amino acid.

The term “elected amino acid”, as used herein, refers to an amino acid residue(s) that has been selected by the methods of the present invention for incorporation as a replacement amino acid at a candidate amino acid position within a polypeptide. In one embodiment, substitution of a candidate amino acid residue position with an elected amino acid residue either reduces or increases the electrostatic contribution to binding free energy of the Fc-FcR complex.

The term “antibody” as used herein includes a naturally occurring antibody obtained from, or produced by, animals that generate antibodies. For example, the antibody can be an antibody produced by, or obtained from, a rodent such as a mouse, rat, gerbil, hamster or guinea pig; from a larger animal such as a rabbit, cat or dog; from an animal commonly kept as livestock (e.g., a pig, a cow, a horse, a sheep, or a goat); or from a primate (including human and non-human primates). The term “antibody” also includes immunoglobulin molecules and modified immunoglobulin molecules, e.g., molecules that contain an antigen binding site which binds (immunoreacts with) an antigen and at least a portion of the Fc region that mediates binding to FcR. As used herein, the term “antibody” also includes modified or synthetic antibody molecules which comprise at least a portion of a Fc region.

As used herein, the term “hinge region” includes the portion of a heavy chain molecule that joins the CHI domain to the CH2 domain, e.g. from about position 216-230 according to the EU number system. This hinge region comprises approximately 25 residues and is flexible, thus allowing the two N-terminal antigen binding regions to move independently. Hinge regions can be subdivided into three distinct domains: upper, middle, and lower hinge domains (Roux et al. J. Immunol. 1998 161:4083).

As used herein, the term “CH2 domain” includes the portion of a heavy chain molecule that extends, e.g., from about EU positions 231-340. The CH2 domain is unique in that it is not closely paired with another domain. Rather, two N-linked branched carbohydrate chains are interposed between the two CH2 domains of an intact native IgG molecule.

As used herein, the term “CH3 domain” includes the portion of a heavy chain molecule that extends approximately 110 residues from N-terminus of the CH2 domain, e.g., from about residue 341-446, EU numbering system). The CH3 domain typically forms the C-terminal portion of the antibody. In some immunoglobulins, however, additional domains may extend from CH3 domain to form the C-terminal portion of the molecule (e.g. the CH4 domain in the 1 chain of IgM and the ε chain of IgE).

“Computational analysis” as referred to herein, refers to a computer implemented process which performs all or some the operations described herein. Such a process will include an output device that displays information to a user (e.g., a CRT display, an LCD, a printer, a communication device such as a modem, audio output, and the like). The computer-implemented process is not limited to a particular computer platform, particular processor, or particular high-level programming language.

The term “structure”, or “structural data”, as used herein, includes the known, predicted and/or modeled position(s) in three-dimensional space that are occupied by the atoms, molecules, compounds, amino acid residues and portions thereof, and macromolecules and portions thereof, of the invention, and, in particular, a polypeptide bound to an antigen in a solvent. A number of methods for identifying and/or predicting structure at the molecular/atomic level can be used such as X-ray crystallography, NMR structural modeling, and the like.

The phrase “spatial representation of an optimal charge distribution”, as used herein, includes modeling the charge distribution for an Fc region or Fc-FcR complex, wherein the electrostatic contribution to free energy of the antibody when bound to antigen is optimized (minimized), as compared to the known and/or modeled representation of charge distribution of the starting polypeptide and/or starting polypeptide when bound to FcR. The modeling of optimal charge distribution can be arrived at by an in silico process that incorporates the known and/or modeled structure(s) of an Fc region or Fc-FcR complex as an input. Response continuum modeling (e.g., the linearized Poisson-Boltzmann equation) can be employed to express the electrostatic binding free energy of the complex in a solvent as a sum of Fc desolvation, Fc-FcR interaction, and FcR desolvation terms. This in silico process is characterized by the ability to incorporate monopole, dipolar, and quadrupolar terms in representing charge distributions within the modeled charge distributions of the invention, and allows for extensive assessment of solvation/desolvation energies for amino acid residues of a polypeptide during transition of the Fc region or portion thereof between unbound and bound states. The process of modeling the spatial representation of an optimal charge distribution for a antibody-antigen complex may additionally incorporate modeling of van der Waals forces, solvent accessible surface area forces, etc.

The term “solvent”, as used herein, includes its broadest art-recognized meaning, referring to any liquid in which a polypeptide of the instant invention is dissolved and/or resides. Preferably, the solvent is a biologically compatable solvent. Preferred solvents include PBS, serum, and the like.

Preferred starting polypeptides comprise an amino acid sequence derived from a human Fc region. A polypeptide or amino acid sequence “derived from” a designated polypeptide or source species refers to the origin of the polypeptide. Preferably, the polypeptide or amino acid sequence which is derived from a particular starting polypeptide or amino acid sequence has an amino acid sequence that is essentially identical to that of the starting sequence, or a portion thereof wherein the portion consists of at least 10-20 amino acids, preferably at least 20-30 amino acids, more preferably at least 30-50 amino acids, or which is otherwise identifiable to one of ordinary skill in the art as having its origin in the starting sequence. For example, polypeptides derived from human polypeptides may comprise one or more amino acids from another mammalian species. For example, a primate Fc domain, hinge portion, or binding site may be included in the subject polypeptides. Alternatively, one or more murine amino acids may be present in a starting polypeptide, e.g., in an antigen binding site (CDR) of an antibody. Preferred starting polypeptides of the invention are not immunogenic.

The term “PEGylation moiety”, “polyethylene glycol moiety”, or “PEG moiety” includes a polyalkylene glycol compound or a derivative thereof, with or without coupling agents or derviatization with coupling or activating moieties (e.g., with thiol, triflate, tresylate, azirdine, oxirane, or preferably with a maleimide moiety, e.g., PEG-maleimide). Other appropriate polyalkylene glycol compounds include, but are not limited to, maleimido monomethoxy PEG, activated PEG polypropylene glycol, but also charged or neutral polymers of the following types: dextran, colominic acids, or other carbohydrate based polymers, polymers of amino acids, and biotin derivatives.

The term “functional moiety” includes moieties which, preferably, add a desirable function to the variant polypeptide. Preferably, the function is added without significantly altering an intrinsic desirable activity of the polypeptide, e.g., in the case of an antibody, the antigen-binding activity of the molecule. A variant polypeptide of the invention may comprise one or more functional moieties, which may be the same or different. Examples of useful functional moieties include, but are not limited to, a PEGylation moiety, a blocking moiety, detectable moiety, a diagnostic moiety, and a therapeutic moiety. Exemplary detectable moieties include fluorescent moieties, radioisotopic moieties, radiopaque moieties, and the like. Exemplary diagnostic moieties include moieties suitable for revealing the presence of an indicator of a disease or disorder. Exemplary therapeutic moieties include, for example, anti-inflammatory agents, anti-cancer agents, anti-neurodegenerative agents, and anti-infective agents. The functional moiety may also have one or more of the above-mentioned functions. Other useful functional moieties are known in the art and described, below.

As used herein, the terms “anti-cancer agent” or “chemotherapeutic agent” includes agents which are detrimental to the growth and/or proliferation of neoplastic or tumor cells and may act to reduce, inhibit or destroy malignancy. Examples of such agents include, but are not limited to, cytostatic agents, alkylating agents, antibiotics, cytotoxic nucleosides, tubulin binding agents, hormones and hormone antagonists, and the like. Any agent that acts to retard or slow the growth of irnmunoreactive cells or malignant cells is within the scope of the present invention.

The term “vector” or “expression vector” is used herein for the purposes of the specification and claims, to mean vectors used in accordance with the present invention as a vehicle for introducing into and expressing a desired polynucleotide in a cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

The term “host cell” refers to a cell that has been transformed with a vector constructed using recombinant DNA techniques and encoding at least one heterologous gene. In descriptions of processes for isolation of proteins from recombinant hosts, the terms “cell” and “cell culture” are used interchangeably to denote the source of protein unless it is clearly specified otherwise. In other words, recovery of protein from the “cells” may mean either from spun down whole cells, or from the cell culture containing both the medium and the suspended cells.

As used herein, “tumor-associated antigens” means any antigen which is generally associated with tumor cells, i.e., occurring at the same or to a greater extent as compared with normal cells. Such antigens may be relatively tumor specific and limited in their expression to the surface of malignant cells, although they may also be found on non-malignant cells. In one embodiment, the altered polypeptides of the present invention bind to a tumor-associated antigen. Accordingly, the starting polypeptides of the present invention may be derived, generated or fabricated from any one of a number of antibodies that react with tumor associated molecules.

As used herein, the term “malignancy” refers to a non-benign tumor or a cancer. As used herein, the term “cancer” includes a malignancy characterized by deregulated or uncontrolled cell growth. Exemplary cancers include: carcinomas, sarcomas, leukemias, and lymphomas. The term “cancer” includes primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor).

As used herein, the phrase “subject that would benefit from administration of a polypeptide” includes subjects, such as mammalian subjects, that would receive a positive therapeutic or prophylactic outcome from administration of a polypeptide of the invention. Exemplary beneficial uses of the polypeptides disclosed herein include, e.g., detection of an antigen recognized by a polypeptide (e.g., for a diagnostic procedure) or treatment with a polypeptide to reduce or eliminate the target recognized by the polypeptide. For example, in one embodiment, the subject may benefit from reduction or elimination of a soluble or particulate molecule from the circulation or serum (e.g., a toxin or pathogen) or from reduction or elimination of a population of cells expressing the target (e.g., tumor cells). As described in more detail herein, the polypeptide can be used in unconjugated form or can be conjugated, e.g., to a drug, prodrug, tag, or an isotope.

II. Fc Containing Polypeptides for Modification

In one embodiment, a starting polypeptide of the invention comprises at least a portion of an Fc region sufficient to confer FcR binding. The portion of the Fc region that binds to FcR comprises from about amino acids 231-446 of IgG1, EU numbering. Amino acid positions in the Fc region are numbered herein according to the EU index numbering system (see Kabat et al., in “Sequences of Proteins of Immunological Interest”, U.S. Dept. Health and Human Services, 5^{th} edition, 1991). The “EU index as in Kabat” refers to the residue numbering of the human IgG1 EU antibody.

Fc regions of the invention are preferably human in origin. A nucleotide sequence encoding the Fc region of the CB6 antibody (comprising a human IgG1 region) is shown in SEQ ID NO:1 and the amino acid sequence encoded by the nucleotide sequence of SEQ ID NO:1 is shown in SEQ ID NO:2. The amino acid sequence of the Fc region is also presented below in Table 1 to illustrate the EU numbering of the amino acids.

TABLE 1

CB6 Amino acid Sequence in EU numbering and
indicating CH2 and CH3 domains.

CH2 domain (EU Positions 231-340)

| | |
|---|---|
| 231 | APELLGG |
| 238 | PSVFLFPPKP |
| 248 | KDTLMISRTP |
| 258 | EVTCVVVDVS |
| 268 | HEDPEVKFNW |
| 278 | YVDGVEVHNA |
| 288 | KTKPREEQYN |
| 298 | STYRVVSVLT |
| 308 | VLHQDWLNGK |
| 318 | EYKCKVSNKA |
| 328 | LPAPIEKTIS |
| 338 | KAK |

CH3 domain (EU positions 341-446)

| | |
|---|---|
| 341 | GQPREPQ |
| 348 | VYTLPPSRDE |
| 358 | LTKNQVSLTC |
| 368 | LVKGFYPSDI |
| 378 | AVEWESNGQP |
| 388 | ENNYKTTPPV |
| 398 | LDSDGSFFLY |
| 408 | SKLTVDKSRW |
| 418 | QQGNVFSCSV |
| 428 | MHEALHNHYT |
| 438 | QKSLSLSPG |

In one embodiment, a starting polypeptide of the invention comprises at least amino acids 231-436 of an Fc region (a complete CH2 domain and a complete CH3 domain). In another embodiment, a starting polypeptide of the invention comprises at least a complete CH2 domain (about amino acids 231-340 of an antibody Fc region according to EU numbering), a complete CH3 domain (about amino acids 341-436 of an antibody Fc region according to EU numbering) and a complete hinge region (about amino acids 216-230 of an antibody Fc region according to EU numbering).

In one embodiment, a starting polypeptide of the invention comprises the sequencece shown in SEQ ID NO:2. Fc regions or FcR binding portions thereof may be derived from heavy chains of any isotype, including IgG1, IgG2, IgG3 and IgG4. In one embodiment, the human isotype IgG1 is used.

The domains making up the Fc region of a starting polypeptide may be derived from different immunoglobulin molecules. For example, a polypeptide may comprise a CH2 domain derived from an IgG1 molecule and a hinge region derived from an IgG3 molecule. In another example, a starting polypeptide can comprise a hinge region derived, in part, from an IgG1 molecule and, in part, from an IgG3 molecule. In another example, a starting polypeptide can comprise a chimeric hinge derived, in part, from an IgG1 molecule and, in part, from an IgG4 molecule. As set forth above, it will be understood by one of ordinary skill in the art that the starting Fc domains may be modified (e.g., in a non-FcR binding portion of the molecule) such that they vary in amino acid sequence from a naturally occurring antibody molecule.

The starting polypeptides of the invention may comprise at least one Fc region or FcR binding portion thereof. Preferred starting polypeptides of the invention additionally comprise at least one binding domain, e.g., an antigen binding domain, receptor binding domain, or ligand binding domain. In one embodiment, the starting polypeptides comprise at least one binding domain and at least one Fc portion. In one embodiment, the starting polypeptide is comprised of two binding domains and two Fc portion.

In one embodiment, the starting polypeptides of the invention have at least one binding domain specific for a target molecule which mediates a biological effect (e.g., a ligand capable of binding to a cell surface receptor or a cell surface receptor capable of binding a ligand) and mediating transmission of a negative or positive signal to a cell together with at least one Fc portion. In one embodiment, starting polypeptides have at least one binding domain specific for an antigen targeted for reduction or elimination, e.g., a cell surface antigen or a soluble antigen, together with at least one Fc region or FcR binding portion thereof.

A. Antibodies

In one embodiment, a starting polypeptide of the invention is an antibody. Using art recognized protocols, for example, antibodies are preferably raised in mammals by multiple subcutaneous or intraperitoneal injections of the relevant antigen (e.g., purified tumor associated antigens or cells or cellular extracts comprising such antigens) and an adjuvant. This immunization typically elicits an immune response that comprises production of antigen-reactive antibodies from activated splenocytes or lymphocytes.

In embodiments in which the Fc containg polypeptide is an antibody, the antibody can be a monoclonal or polyclonal antibody. Methods for producing monoclonal antibodies have been known for some time (see, e.g., Kohler and Milstein, Nature 256:495-497, 1975), as have techniques for stably introducing immunoglobulin-encoding DNA into myeloma cells (see, e.g., Oi et al., Proc. Natl. Acad. Sci. USA 80:6351-6355, 1983). These techniques, which include in vitro mutagenesis and DNA transfection, allow the construction of recombinant immunoglobulins and can be used to produce the polypeptide used in the methods of the invention or those that result therefrom (e.g., therapeutic and diagnostic antibodies). Production methods, vectors, and hosts are described further below.

The starting antibodies used in the invention may be produced in a non-human mammal, e.g., murine, guinea pig, primate, rabbit or rat, by immunizing the animal with the antigen or a fragment thereof. See Harlow & Lane, supra, incorporated by reference for all purposes. While the resulting antibodies may be harvested from the serum of the animal to provide polyclonal preparations, it is often desirable to isolate individual lymphocytes from the spleen, lymph nodes or peripheral blood to provide homogenous preparations of monoclonal antibodies (MAbs). Rabbits or guinea pigs are typically used for making polyclonal antibodies. Mice are typically used for making monoclonal antibodies. Monoclonal antibodies can be prepared against a fragment by injecting an antigen fragment into a mouse, preparing “hybridomas” and screening the hybridomas for an antibody that specifically binds to the antigen. In this well known process (Kohler et al., (1975), Nature, 256:495) the relatively short-lived, or mortal, lymphocytes from the mouse which has been injected with the antigen are fused with an immortal tumor cell line (e.g. a myeloma cell line), thus, producing hybrid cells or “hybridomas” which are both immortal and capable of producing the genetically coded antibody of the B cell. The resulting hybrids are segregated into single genetic strains by selection, dilution, and regrowth with each individual strain comprising specific genes for the formation of a single antibody. They produce antibodies which are homogeneous against a desired antigen and, in reference to their pure genetic parentage, are termed “monoclonal”.

Hybridoma cells thus prepared are seeded and grown in a suitable culture medium that preferably contains one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. Preferably, the binding specificity of the monoclonal antibodies produced by hybridoma cells is determined by immunoprecipitation or by an in vitro assay, such as a radioimmunoassay (RIA) or enzyme-linked immunoabsorbent assay (ELISA). After hybridoma cells are identified that produce antibodies of the desired specificity, affinity and/or activity, the clones may be subcloned by limiting dilution procedures and grown by standard methods (Goding, Monoclonal Antibodies: Principles and Practice, pp 59-103 (Academic Press, 1986)). It will further be appreciated that the monoclonal antibodies secreted by the subclones may be separated from culture medium, ascites fluid or serum by conventional purification procedures such as, for example, protein-A, hydroxylapatite chromatography, gel electrophoresis, dialysis or affinity chromatography.

Optionally, antibodies may be screened for binding to a specific region or desired fragment of the antigen without binding to other nonoverlapping fragments of the antigen. The latter screening can be accomplished by determining binding of an antibody to a collection of deletion mutants of the antigen and determining which deletion mutants bind to the antibody. Binding can be assessed, for example, by Western blot or ELISA. The smallest fragment to show specific binding to the antibody defines the epitope of the antibody. Alternatively, epitope specificity can be determined by a competition assay is which a test and reference antibody compete for binding to the antigen. If the test and reference antibodies compete, then they bind to the same epitope or epitopes sufficiently proximal such that binding of one antibody interferes with binding of the other.

In another embodiment, DNA encoding the desired monoclonal antibodies may be readily isolated and sequenced using conventional procedures (e.g., by using oligonucleotide probes that are capable of binding specifically to genes encoding the heavy and light chains of murine antibodies). The isolated and subcloned hybridoma cells serve as a preferred source of such DNA. Once isolated, the DNA may be placed into expression vectors, which are then transfected into prokaryotic or eukaryotic host cells such as E. coli cells, simian COS cells, Chinese Hamster Ovary (CHO) cells or myeloma cells that do not otherwise produce immunoglobulins. More particularly, the isolated DNA (which may be synthetic as described herein) may be used to clone constant and variable region sequences for the manufacture antibodies as described in Newman et al., U.S. Pat. No. 5,658,570, filed Jan. 25, 1995, which is incorporated by reference herein. Essentially, this entails extraction of RNA from the selected cells, conversion to cDNA, and amplification by PCR using Ig specific primers. Suitable primers for this purpose are also described in U.S. Pat. No. 5,658,570. As will be discussed in more detail below, transformed cells expressing the desired antibody may be grown up in relatively large quantities to provide clinical and commercial supplies of the immunoglobulin.

Those skilled in the art will also appreciate that DNA encoding antibodies or antibody fragments (e.g., antigen binding sites) may also be derived from antibody phage libraries, e.g., using pd phage or Fd phagemid technology. Exemplary methods are set forth, for example, in EP 368 684 Bi; U.S. Pat. No. 5,969,108, Hoogenboom, H. R. and Chames. 2000. Immunol. Today 21:371; Nagy et al. 2002. Nat. Med. 8:801; Huie et al. 2001. Proc. Natl. Acad. Sci. USA 98:2682; Lui et al. 2002. J. Mol. Biol. 315:1063, each of which is incorporated herein by reference. Several publications (e.g., Marks et al. Bio/Technology 10:779-783 (1992)) have described the production of high affinity human antibodies by chain shuffling, as well as combinatorial infection and in vivo recombination as a strategy for constructing large phage libraries. In another embodiment, Ribosomal display can be used to replace bacteriophage as the display platform (see, e.g., Hanes et al. 2000. Nat. Biotechnol. 18:1287; Wilson et al. 2001. Proc. Natl. Acad. Sci. USA 98:3750; or Irving et al. 2001 J. Immunol. Methods 248:31. In yet another embodiment, cell surface libraries can be screened for antibodies (Boder et al. 2000. Proc. Natl. Acad. Sci. USA 97:10701; Daugherty et al. 2000 J. Immunol. Methods 243:211. Such procedures provide alternatives to traditional hybridoma techniques for the isolation and subsequent cloning of monoclonal antibodies.

Yet other embodiments of the present invention comprise the generation of human or substantially human antibodies in transgenic animals (e.g., mice) that are incapable of endogenous immunoglobulin production (see e.g., U.S. Pat. Nos. 6,075,181, 5,939,598, 5,591,669 and 5,589,369, each of which is incorporated herein by reference). For example, it has been described that the homozygous deletion of the antibody heavy-chain joining region in chimeric and germ-line mutant mice results in complete inhibition of endogenous antibody production. Transfer of a human immunoglobulin gene array to such germ line mutant mice will result in the production of human antibodies upon antigen challenge. Another preferred means of generating human antibodies using SCID mice is disclosed in U.S. Pat. No. 5,811,524 which is incorporated herein by reference. It will be appreciated that the genetic material associated with these human antibodies may also be isolated and manipulated as described herein.

Yet another highly efficient means for generating recombinant antibodies is disclosed by Newman, Biotechnology, 10: 1455-1460 (1992). Specifically, this technique results in the generation of primatized antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

In another embodiment, lymphocytes can be selected by micromanipulation and the variable genes isolated. For example, peripheral blood mononuclear cells can be isolated from an immunized mammal and cultured for about 7 days in vitro. The cultures can be screened for specific IgGs that meet the screening criteria. Cells from positive wells can be isolated. Individual Ig-producing B cells can be isolated by FACS or by identifying them in a complement-mediated hemolytic plaque assay. Ig-producing B cells can be micromanipulated into a tube and the VH and VL genes can be amplified using, e.g., RT-PCR. The VH and VL genes can be cloned into an antibody expression vector and transfected into cells (e.g., eukaryotic or prokaryotic cells) for expression.

Moreover, genetic sequences usefuil for producing the polypeptides of the present invention may be obtained from a number of different sources. For example, as discussed extensively above, a variety of human antibody genes are available in the form of publicly accessible deposits. Many sequences of antibodies and antibody-encoding genes have been published and suitable antibody genes can be chemically synthesized from these sequences using art recognized techniques. Oligonucleotide synthesis techniques compatible with this aspect of the invention are well known to the skilled artisan and may be carried out using any of several commercially available automated synthesizers. In addition, DNA sequences encoding several types of heavy and light chains set forth herein can be obtained through the services of commercial DNA synthesis vendors. The genetic material obtained using any of the foregoing methods may then be altered or synthetic to provide obtain polypeptides of the present invention.

Variable and constant domains can be separately cloned, e.g., using the polymerase chain reaction and primers which are selected to amplify the domain of interest. In addition, the sequences of many antibody variable and constant domains are known and such domains can be synthesized using methods well known in the art. For example, constant region domains can be selected having a particular effector function (or lacking a particular effector function) or with a particular modification to reduce immunogenicity. Alternatively, variable domains can be obtained from libraries of variable gene sequences from an animal of choice. Libraries expressing random combinations of domains, e.g., V_{H} and V_{L} domains, can be screened with a desired antigen to identify elements which have desired binding characteristics. Methods of such screening are well known in the art. For example, antibody gene repertoires can be cloned into a λ bacteriophage expression vector (Huse, W D et al. (1989). Science, 2476:1275). In addition, cells (Francisco et al. (1994), PNAS, 90:10444; Georgiou et al. (1997), Nat. Biotech., 15:29; Boder and Wittrup (1997) Nat. Biotechnol.. 15:553; Boder et al.(2000), PNAS, 97:10701; Daugtherty, P. et al. (2000) J. Immunol.. Methods. 243:211) or viruses (e.g., Hoogenboom, H R. (1998), Immunotechnology 4:1; Winter et al. (1994). Annu. Rev. Immunol.. 12:433; Griffiths, A D. (1998). Curr. Opin. Biotechnol. 9:102) expressing antibodies on their surface can be screened. Those skilled in the art will also appreciate that DNA encoding antibody domains may also be derived from antibody phage libraries, e.g., using pd phage or Fd phagemid technology. Exemplary methods are set forth, for example, in EP 368 684 B1; U.S. Pat. No. 5,969,108; Hoogenboom et al., (2000) Immunol. Today 21:371; Nagy et al. (2002) Nat. Med. 8:801; Huie et al. (2001), PNAS, 98:2682; Lui et al. (2002), J. MoL Biol. 315:1063, each of which is incorporated herein by reference. Several publications (e.g., Marks et al. (1992), Bio/Technology 10:779-783) have described the production of high affinity human antibodies by chain shuffling, as well as combinatorial infection and in vivo recombination as a strategy for constructing large phage libraries. In another embodiment, ribosomal display can be used to replace bacteriophage as the display platform (see, e.g., Hanes, et al. (1998), PNAS 95:14130; Hanes and Pluckthun. (1999), Curr. Top. Microbiol. Immunol. 243:107; He and Taussig. (1997), Nuc. Acids Res., 25:5132; Hanes et al. (2000), Nat. Biotechnol. 18:1287; Wilson et al. (2001), PNAS, 98:3750; or Irving et al. (2001) J. Immunol.. Methods 248:31).

Preferred libraries for screening are human variable gene libraries. V_{L} and V_{H} domains from a non-human source may also be used. Libraries can be naïve, from immunized subjects, or semi-synthetic (Hoogenboom and Winter. (1992). J. Mol. BioL 227:381; Griffiths et al. (1995) EMBO J. 13:3245; de Kruif et al. (1995). J. Mol. Biol. 248:97; Barbas et al. (1992), PNAS, 89:4457). In one embodiment, mutations can be made to immunoglobulin domains to create a library of nucleic acid molecules having greater heterogeneity (Thompson et al. (1996), J. Mol. Biol. 256:77; Lamminmaki et al. (1999), J. Mol. Biol. 291:589; Caldwell and Joyce. (1992), PCR Methods Appl. 2:28; Caldwell and Joyce. (1994), PCR Methods Appl. 3:S136). Standard screening procedures can be used to select high affinity variants. In another embodiment, changes to V_{H} and V_{L} sequences can be made to increase antibody avidity, e.g., using information obtained from crystal structures using techniques known in the art.

Alternatively, antibody-producing cell lines may be selected and cultured using techniques well known to the skilled artisan. Such techniques are described in a variety of laboratory manuals and primary publications. In this respect, techniques suitable for use in the invention as described below are described in Current Protocols in Immunology, Coligan et al., Eds., Green Publishing Associates and Wiley-Interscience, John Wiley and Sons, New York (1991) which is herein incorporated by reference in its entirety, including supplements.

It will further be appreciated that the scope of this invention further encompasses all alleles, variants and mutations of antigen binding DNA sequences.

As is well known, RNA may be isolated from the original hybridoma cells or from other transformed cells by standard techniques, such as guanidinium isothiocyanate extraction and precipitation followed by centrifugation or chromatography. Where desirable, mRNA may be isolated from total RNA by standard techniques such as chromatography on oligo dT cellulose. Suitable techniques are familiar in the art.

In one embodiment, cDNAs that encode the light and the heavy chains of the antibody may be made, either simultaneously or separately, using reverse transcriptase and DNA polymerase in accordance with well known methods. PCR may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes.

DNA, typically plasmid DNA, may be isolated from the cells using techniques known in the art, restriction mapped and sequenced in accordance with standard, well known techniques set forth in detail, e.g., in the foregoing references relating to recombinant DNA techniques. Of course, the DNA may be synthetic according to the present invention at any point during the isolation process or subsequent analysis. In many cases imnuunoreative antibodies for each of these antigens have been reported in the literature.

In another embodiment, binding of the starting polypeptide to an antigen results in the reduction or elimination of the antigen, e.g., from a tissue or from the circulation. In another embodiment, the starting polypeptide has at least one binding domain specific for an antigen that can be used to detect the presence of a target molecule (e.g., to detect a contaminant or diagnose a condition or disorder). In yet another embodiment, a starting polypeptide of the invention comprises at least one binding site that targets the molecule to a specific site in a subject (e.g., to a tumor cell or blood clot).

In one embodiment, the starting polypeptides of the present invention may be immunoreactive with one or more tumor-associated antigens. For example, for treating a cancer or neoplasia an antigen binding domain of a polypeptide preferably binds to a selected tumor associated antigen. Given the number of reported antigens associated with neoplasias, and the number of related antibodies, those skilled in the art will appreciate that a polypeptide of the invention may be derived from any one of a number of whole antibodies. More generally, starting antibodies useful in the present invention may be obtained or derived from any antibody (including those previously reported in the literature) that reacts with an antigen or marker associated with the selected condition. Further, a starting antibody, or fragment thereof, used to generate the disclosed polypeptides may be murine, human, chimeric, humanized, non-human primate or primatized. Exemplary tumor-associated antigens bound by the starting polypeptides used in the invention include for example, pan B antigens (e.g. CD20 found on the surface of both malignant and non-malignant B cells such as those in non-Hodgkin's lymphoma) and pan T cell antigens (e.g. CD2, CD3, CD5, CD6, CD7). Other exemplary tumor associated antigens comprise but are not limited to MAGE-1, MAGE-3, MUC-1, HPV 16, HPV E6 & E7, TAG-72, CEA, α-Lewis^{y}, L6-Antigen, CD19, CD22, CD25, CD30, CD33, CD37, CD44, CD52, CD56, mesothelin, PSMA, HLA-DR, EGF Receptor, VEGF Receptor, and HER2 Receptor.

Previously reported antibodies that react with tumor-associated antigens may be altered as described herein to provide the altered antibodies of the present invention. Exemplary target antibodies capable of reacting with tumor-associated antigens inclue: 2B8, Lym 1, Lym 2, LL2, Her2, B1, BR96, MB1, BH3, B4, B72.3, 5E8, B3F6, 5E10, α-CD33, α-CanAg, α-CD56, α-CD44v6, α-Lewis, and α-CD30.

More specifically, exemplary target antibodies include, but are not limited to 2B8 and C2B8 (Zevalin® and Rituxan®, IDEC Pharmaceuticals Corp., San Diego), Lym 1 and Lym 2 (Techniclone), LL2 (Immunomedics Corp., New Jersey), Trastuzumab (Herceptin®, Genentech Inc., South San Francisco), Tositumomab (Bexxar®, Coulter Pharm., San Francisco), Alemtzumab (Campath®, Millennium Pharmaceuticals, Cambridge), Gemtuzumab ozogamicin (Mylotarg®, Wyeth-Ayerst, Philadelphia), Cetuximab (Erbitux®, Imclone Systems, New York), Bevacizumab (Avastin®, Genentech Inc., South San Francisco), BR96, BL22, LMB9, LMB2, MB1, BH3, B4, B72.3 (Cytogen Corp.), SSI (NeoPharm), CC49 (National Cancer Institute), Cantuzumab mertansine (ImmunoGen, Cambridge), MNL 2704 (Milleneum Pharmaceuticals, Cambridge), Bivatuzumab mertansine (Boehringer Ingelheim, Germany), Trastuzumab-DM1 (Genentech, South San Francisco), My9-6-DM1 (ImmunoGen, Cabridge), SGN-10, -15, -25, and -35 (Seattle Genetics, Seattle), and 5E10 (University of Iowa). In preferred embodiments, the starting antibodies of the present invention will bind to the same tumor-associated antigens as the antibodies enumerated immediately above. In particularly preferred embodiments, the polypeptides will be derived from or bind the same antigens as Y2B8, C2B8, CC49 and C5E10.

In a first preferred embodiment, the starting antibody will bind to the same tumor-associated antigen as Rituxan®. Rituxan® (also known as, rituximab, IDEC-C2B8 and C2B8) was the first FDA-approved monoclonal antibody for treatment of human B-cell lymphoma (see U.S. Pat. Nos. 5,843,439; 5,776,456 and 5,736,137 each of which is incorporated herein by reference). Y2B8 (90Y labeled 2B8; Zevalin®; ibritumomab tiuxetan) is the murine starting of C2B8. Rituxan® is a chimeric, anti-CD20 monoclonal antibody which is growth inhibitory and reportedly sensitizes certain lymphoma cell lines for apoptosis by chemotherapeutic agents in vitro. The antibody efficiently binds human complement, has strong FcR binding, and can effectively kill human lymphocytes in vitro via both complement dependent (CDC) and antibody-dependent (ADCC) mechanisms (Reffet al., Blood 83: 435-445 (1994)). Those skilled in the art will appreciate that dimeric variants (homodimers or heterodimers) of C2B8 or 2B8, synthetic according to the instant disclosure, may be conjugated with effector moieties according to the methods of the invention, in order to provide modified antibodies with even more effective in treating patients presenting with CD20+ malignancies.

In other preferred embodiments of the present invention, the starting polypeptide of the invention will be derived from, or bind to, the same tumor-associated antigen as CC49. CC49 binds human tumor-associated antigen TAG-72 which is associated with the surface of certain tumor cells of human origin, specifically the LS174T tumor cell line. LS174T [American Type Culture Collection (herein ATCC) No. CL 188] is a variant of the LS180 (ATCC No. CL 187) colon adenocarcinoma line.

It will further be appreciated that numerous murine monoclonal antibodies have been developed which have binding specificity for TAG-72. One of these monoclonal antibodies, designated B72.3, is a murine IgG1 produced by hybridoma B72.3 (ATCC No. HB-8108). B72.3 is a first generation monoclonal antibody developed using a human breast carcinoma extract as the immunogen (see Colcher et al., Proc. Natl. Acad. Sci. (USA), 78:3199-3203 (1981); and U.S. Pat. Nos. 4,522,918 and 4,612,282 each of which is incorporated herein by reference). Other monoclonal antibodies directed against TAG-72 are designated “CC” (for colon cancer). As described by Schlom et al. (U.S. Pat. No. 5,512,443 which is incorporated herein by reference) CC monoclonal antibodies are a family of second generation murine monoclonal antibodies that were prepared using TAG-72 purified with B72.3. Because of their relatively good binding affinities to TAG-72, the following CC antibodies have been deposited at the ATCC, with restricted access having been requested: CC49 (ATCC No. HB 9459); CC 83 (ATCC No. HB 9453); CC46 (ATCC No. HB 9458); CC92 (ATTCC No. HB 9454); CC30 (ATCC No. BB 9457); CC11 (ATCC No. 9455); and CC15 (ATCC No. HB 9460). U.S. Pat. No. 5,512,443 further teaches that the disclosed antibodies may be altered into their chimeric form by substituting, e.g., human constant regions (Fc) domains for mouse constant regions by recombinant DNA techniques known in the art. Besides disclosing murine and chimeric anti-TAG-72 antibodies, Schlom et al. have also produced variants of a humanized CC49 antibody as disclosed in PCT/US99/25552 and single chain constructs as disclosed in U.S. Pat. No. 5,892,019 each of which is also incorporated herein by reference. Those skilled in the art will appreciate that each of the foregoing antibodies, constructs or recombinants, and variations thereof, may be synthetic and used to provide polypeptides in accordance with the present invention.

In addition to the anti-TAG-72 antibodies discussed above, various groups have also reported the construction and partial characterization of domain-deleted CC49 and B72.3 antibodies (e.g., Calvo et al. Cancer Biotherapy, 8(1):95-109 (1993), Slavin-Chiorini et al. Int. J. Cancer 53:97-103 (1993) and Slavin-Chiorini et al. Cancer. Res. 55:5957-5967 (1995).

In one embodiment, a starting polypeptide of the invention binds to the CD23 (U.S. Pat. No. 6,011,138). In a preferred embodiment, a starting polypeptide of the invention binds to the same epitope as the 5E8 antibody. In another embodiment, a starting polypeptide of the invention comprises at least one CDR from an anti-CD23 antibody, e.g., the 5E8 antibody.

In a preferred embodiment, a starting polypeptide of the invention binds to the CRIPTO-I antigen (WO02/088170A2 or WO03/083041A2). In a more preferred embodiment, a polypeptide of the invention binds to the same epitope as the B3F6 antibody. In still another embodiment, a polypeptide of the invention comprises at least one CDR from an anti-CRIPTO-I antibody, e.g., the B3F6 antibody.

Still other embodiments of the present invention comprise modified antibodies that are derived from or bind to the same tumor associated antigen as C5E10. As set forth in co-pending application Ser. No. 09/104,717, C5E10 is an antibody that recognizes a glycoprotein determinant of approximately 115 kDa that appears to be specific to prostate tumor cell lines (e.g. DU145, PC3, or ND1). Thus, in conjunction with the present invention, polypeptides that specifically bind to the same tumor-associated antigen recognized by C5E10 antibodies could be used alone or conjugated with an effector moiety by the methods of the invention, thereby providing a modified polypeptide that is usefuil for the improved treatment of neoplastic disorders. In particularly preferred embodiments, the starting polypeptide will be derived or comprise all or part of the antigen binding region of the C5E10 antibody as secreted from the hybridoma cell line having ATCC accession No. PTA-865. The resulting polypeptide could then be conjugated to a therapeutic effector moiety as described below and administered to a patient suffering from prostate cancer in accordance with the methods herein.

B. Antibody,Variants

In addition to naturally-occuring antibodies, the starting antibodies of the invention may include immunoreactive fragments or portions which are not naturally occurring.

In another embodiment, a heavy chain variable portion and a light chain variable portion of an antigen binding domain of a target antibody of the invention are present in the same polypeptide, e.g., as in a single chain antibody (ScFv) or a minibody (see e.g., U.S. Pat. No. 5,837,821 or WO 94/09817A1). Minibodies are dimeric molecules made up of two polypeptide chains each comprising an ScFv molecule (a single polypeptide comprising one or more antigen binding sites, e.g., a V_{L} domain linked by a flexible linker to a V_{H} domain fused to a CH3 domain via a connecting peptide). ScFv molecules can be constructed in a V_{H}-linker-V_{L} orientation or V_{L}-linker-V_{H} orientation. The flexible hinge that links the V_{L} and V_{H} domains that make up the antigen binding site preferably comprises from about 10 to about 50 amino acid residues. An exemplary connecting peptide for this purpose is (Gly4Ser)3 (Huston et al. (1988). PNAS, 85:5879). Other connecting peptides are known in the art.

Methods of making single chain antibodies are well known in the art, e.g., Ho et al. (1989), Gene, 77:51; Bird et al. (1988), Science 242:423; Pantoliano et al. (1991), Biochemistry 30:10117;Milenic et al. (1991), Cancer Research, 51:6363; Takkinen et al. (1991), Protein Engineering 4:837. Minibodies can be made by constructing an ScFv component and connecting peptide-CH₃ component using methods described in the art (see, e.g., U.S. Pat. No. 5,837,821 or WO 94/09817A1). These components can be isolated from separate plasmids as restriction fragments and then ligated and recloned into an appropriate vector. Appropriate assembly can be verified by restriction digestion and DNA sequence analysis. In one embodiment, a minibody of the invention comprises a connecting peptide. In one embodiment, the connecting peptide comprises a Gly/Ser linker, e.g., GGGSSGGGSGG.

In another embodiment, a tetravalent minibody can be constructed. Tetravalent minibodies can be constructed in the same manner as minibodies, except that two ScFv molecules are linked using a flexible linker, e.g., having an amino acid sequence (G4S)₄G3AS.

In another embodiment, a starting antibody of the invention comprises a diabody. Diabodies are similar to scFv molecules, but usually have a short (less than 10 and preferably 1-5) amino acid residue linker connecting both variable domains, such that the V_{L} and V_{H} domains on the same polypeptide chain can not interact. Instead, the V_{L} and V_{H} domain of one polypeptide chain interact with the V_{H} and V_{L} domain (respectively) on a second polypeptide chain (WO 02/02781).

In another embodiment, a starting antibody of the invention comprises an immunoreactive fragment or portion thereof (e.g. an scFv molecule, a minibody, a tetravalent minibody, or a diabody) operably linked to an FcR binding portion. In an exemplary embodiment, the FcR binding portion is a complete Fc region.

In another embodiment, at least one antigen binding domain of a starting antibody is catalytic (Shokat and Schultz.(1990). Annu. Rev. Immunol. 8:335). Antigen binding domains with catalytic binding specificities can be made using art recognized techniques (see, e.g., U.S. Pat. No. 6,590,080, U.S. Pat. No. 5,658,753). Catalytic binding specificities can work by a number of basic mechanisms similar to those identified for enzymes to stabilize the transition state, thereby reducing the free energy of activation. For example, general acid and base residues can be optimally positioned for participation in catalysis within catalytic active sites; covalent enzyme-substrate intermediates can be formed; catalytic antibodies can also be in proper orientation for reaction and increase the effective concentration of reactants by at least seven orders of magnitude (Fersht et al., (1968), J. Am. Chem. Soc. 90:5833) and thereby greatly reduce the entropy of a chemical reaction. Finally, catalytic antibodies can convert the energy obtained upon substrate binding to distort the reaction towards a structure resembling the transition state.

Acid or base residues can be brought into the antigen binding site by using a complementary charged molecule as an immunogen. This technique has proved successful for elicitation of antibodies with a hapten containing a positively-charged ammonium ion (Shokat, et al., (1988), Chem. Int. Ed. Engl. 27:269-271). In another approach, antibodies can be elicited to stable compounds that resemble the size, shape, and charge of the transition state of a desired reaction (i.e., transition state analogs). See U.S. Pat. No. 4,792,446 and U.S. Pat. No. 4,963,355 which describe the use of transition state analogues to immunize animals and the production of catalytic antibodies. Both of these patents are hereby incorporated by reference. Such molecules can be administered as part of an immunoconjugate, e.g., with an immunogenic carrier molecule, such as KLH.

In one embodiment, a starting antibody of the invention is bispecific. Bispecific molecules can bind to two different target sites, e.g., on the same target molecule or on different target molecules. For example, in the case of antibodies, bispecific molecules can bind to two different epitopes, e.g., on the same antigen or on two different antigens. Bispecific molecules can be used, e.g., in diagnostic and therapeutic applications. For example, they can be used to immobilize enzymes for use in immunoassays. They can also be used in diagnosis and treatment of cancer, e.g., by binding both to a tumor associated molecule and a detectable marker (e.g., a chelator which tightly binds a radionuclide. Bispecific molecules can also be used for human therapy, e.g., by directing cytotoxicity to a specific target (for example by binding to a pathogen or tumor cell and to a cytotoxic trigger molecule, such as the T cell receptor. Bispecific antibodies can also be used, e.g., as fibrinolytic agents or vaccine adjuvants.

Examples of bispecific binding molecules include those with at least two arms directed against tumor cell antigens; bispecific binding molecules with at least one arm directed against a tumor cell antigen and the at least one arm directed against a cytotoxic trigger molecule (such as anti-CD3/anti-malignant B-cell (1D10), anti-CD3/anti-p185.sup.HER2, anti-CD3/anti-p97, anti-CD3/anti-renal cell carcinoma, anti-CD3/anti-OVCAR-3, anti-CD3/L-D1 (anti-colon carcinoma), anti-CD3/anti-melanocyte stimulating hormone analog, anti-EGF receptor/anti-CD3, anti-CD3/anti-CAMA1, anti-CD3/anti-CD19, anti-CD3/MoV18, anti-neural cell adhesion molecule (NCAM)/anti-CD3, anti-folate binding protein (FBP)/anti-CD3, anti-pan carcinoma associated antigen (AMOC-31)/anti-CD3); bispecific binding molecules with at least one which binds specifically to a tumor antigen and at least one which binds to a toxin (such as anti-saporin/anti-Id-1, anti-CD22/anti-saporin, anti-CD7/anti-saporin, anti-CD38/anti-saporin, anti-CEA/anti-ricin A chain, anti-interferon-.alpha.(IFN-.alpha.)/anti-hybridoma idiotype, anti-CEA/anti-vinca alkaloid); bispecific binding molecules for converting enzyme activated prodrugs (such as anti-CD30/anti-alkaline phosphatase (which catalyzes conversion of mitomycin phosphate prodrug to mitomycin alcohol)); bispecific binding molecules which can be used as fibrinolytic agents (such as anti-fibrin/anti-tissue plasminogen activator (tPA), anti-fibrin/anti-urokinase-type plasminogen activator (uPA)); bispecific binding molecules for targeting immune complexes to cell surface receptors (such as anti-low density lipoprotein (LDL); bispecific binding molecules for use in therapy of infectious diseases (such as anti-CD3/anti-herpes simplex virus (HSV), anti-T-cell receptor:CD3 complex/anti-influenza, anti-Fc.gamma.R/anti-HIV; bispecific binding molecules for tumor detection in vitro or in vivo such as anti-CEA/anti-EOTUBE, anti-CEA/anti-DPTA, anti-p185HER2/anti-hapten); bispecific binding molecules as vaccine adjuvants (see Fanger et al., supra); and bispecific binding molecules as diagnostic tools (such as anti-rabbit IgG/anti-ferritin, anti-horse radish peroxidase (HRP)/anti-hormone, anti-somatostatin/anti-substance P, anti-HRP/anti-FITC, anti-CEA/anti-.beta.-galactosidase (see Nolan et al., supra)). Examples of trispecific antibodies include anti-CD3/anti-CD4/anti-CD37, anti-CD3/anti-CD5/anti-CD37 and anti-CD3/anti-CD8/anti-CD37.

In a preferred embodiment, a bispecific molecule of the invention binds to CRIPTO-I.

Bispecific molecules may be monovalent for each specificity or be multivalent for each specificity. For example, an antibody molecule or fusion protein may comprise one binding site that reacts with a first target molecule and one binding site that reacts with a second target molecule or it may comprise two binding sites that react with a first target molecule and two binding sites that react with a second target molecule. Methods of producing bispecific molecules are well known in the art. For example, recombinant technology can be used to produce bispecific molecules. Exemplary techniques for producing bispecific molecules are known in the art (e.g., Kontermann et al. Methods in Molecular Biology Vol. 248: Antibody Engineering: Methods and Protocols. Pp227-242 US 2003/0207346 A1 and the references cited therein). In one embodiment, a multimeric bispecific molecules are prepared using methods such as those described e.g., in US 2003/0207346 A1 or U.S. Pat. No. 5,821,333, or US2004/0058400.

As used herein the phrase “multispecific fusion protein” designates fusion proteins (as hereinabove defined) having at least two binding specificities (i.e. combining two or more binding domains of a ligand or receptor). Multispecific fusion proteins can be assembled as heterodimers, heterotrimers or heterotetramers, essentially as disclosed in WO 89/02922 (published Apr. 6, 1989), in EP 314, 317 (published May 3, 1989), and in U.S. Pat. No. 5,116,964 issued May 2, 1992. Preferred multispecific fusion proteins are bispecific. Examples of bispecific fusion proteins include CD4-IgG/TNFreceptor-IgG and CD4-IgG/L-selectin-IgG. The last mentioned molecule combines the lymph node binding function of the lymphocyte homing receptor (LHR, L-selectin), and the HIV binding function of CD4, and finds potential application in the prevention or treatment of HIV infection, related conditions, or as a diagnostic.

Target binding sites for the multispecific binding molecules of the invention can readily be selected by one of ordinary skill in the art. While not limiting in any way, exemplary binding sites include one or more epitopes of a tumor antigen. Other exemplary target molecules include one or more epitopes of, e.g., heparin sulfate, growth factors or their receptors (e.g., epidermal growth factor receptor, insulin-like growth factor receptor, hepatocyte growth factor (HGF/SF) receptor (See, e.g., Cao et al. Proc. Natl. Acad. Sci 2001. 98:7443; Lu et al. 2004. J. Biol. Chem. 279:2856).

In another embodiment, an antigen binding domain of a starting antibody consists of a V_{H} domain, e.g., derived from camelids, which is stable in the absence of a V_{L} chain (Hamers-Casterman et al. (1993). Nature, 363:446; Desmyter et al. (1996). Nat. Struct. Biol. 3: 803; Decanniere et al. (1999). Structure, 7:361; Davies et al. (1996). Protein Eng., 9:531; Kortt et al. (1995). J. Protein Chem., 14:167).

Non-human starting antibodies, or fragments or domains thereof, can be altered to reduce their immunogenicity using art recognized techniques. Humanized starting polypeptides are starting polypeptides derived from a non-human protein, that retains or substantially retains the properties of the starting antibody, but which is less immunogenic in humans. In the case of humanized starting antibodies, this may be achieved by various methods, including (a) grafting the entire non-human variable domains onto human constant regions to generate chimeric target antibodies; (b) grafting at least a part of one or more of the non-human complementarity determining regions (CDRs) into a human framework and constant regions with or without retention of critical framework residues; (c) transplanting the entire non-human variable domains, but “cloaking” them with a human-like section by replacement of surface residues. Such methods are disclosed in Morrison et al., (1984), PNAS. 81: 6851-5; Morrison et al., (1988), Adv. Immunol. 44: 65-92; Verhoeyen et al., (1988), Science 239: 1534-1536; Padlan, (1991), Molec. Immun. 28: 489-498; Padlan, (1994), Molec. Immun. 31: 169-217; and U.S. Pat. Nos. 5,585,089, 5,693,761 and 5,693,762 all of which are hereby incorporated by reference in their entirety.

De-immunization can also be used to decrease the immunogenicity of a starting antibody. As used herein, the term “de-immunization” includes alteration of an antibody to modify T cell epitopes (see, e.g., WO9852976A1, WO0034317A2). For example, VH and VL sequences from the starting antibody are analyzed and a human T cell epitope “map” from each V region showing the location of epitopes in relation to complementarity-determining regions (CDRs) and other key residues within the sequence Individual T cell epitopes from the T cell epitope map are analyzed in order to identify alternative amino acid substitutions with a low risk of altering activity of the antibody. A range of alternative VH and VL sequences are designed comprising combinations of amino acid substitutions and these sequences are subsequently incorporated into a range of polypeptides of the invention that are tested for function. Typically, between 12 and 24 variant antibodies are generated and tested. Complete heavy and light chain genes comprising modified V and human C regions are then cloned into expression vectors and the subsequent plasmids introduced into cell lines for the production of whole antibody. The antibodies are then compared in appropriate biochemical and biological assays, and the optimal variant is identified.

In one embodiment, the polypeptide comprises a chimeric antibody. In the context of the present application the term “chimeric antibodies” will be held to mean any antibody wherein the immunoreactive region or site is obtained or derived from a first species and the constant region (which may be intact, partial or modified in accordance with the instant invention) is obtained from a second species. In preferred embodiments the target binding region or site will be from a non-human source (e.g. mouse) and the constant region is human. Preferably, the variable domains in both the heavy and light chains of a target antibody are altered by at least partial replacement of one or more CDRs and, if necessary, by partial framework region replacement and sequence changing. Although the CDRs may be derived from an antibody of the same class or even subclass as the target antibody from which the framework regions are derived, it is envisaged that the CDRs will be derived from an antibody of different class and preferably from an antibody from a different species. It may not be necessary to replace all of the CDRs with the complete CDRs from the donor variable region to transfer the antigen binding capacity of one variable domain to another. Rather, it may only be necessary to transfer those residues that are necessary to maintain the activity of the binding domain. Given the explanations set forth in U.S. Pat. Nos. 5,585,089, 5,693,761 and 5,693,762, it will be well within the competence of those skilled in the art, either by carrying out routine experimentation or by trial and error testing to obtain a functional antibody with reduced immunogenicity.

In preferred embodiments, a starting polypeptide of the invention will not elicit a deleterious immune response in a human. Those skilled in the art will appreciate that chimeric starting polypeptides can also be produced. In the context of the present application the term “chimeric starting antibody” will be held to mean any starting antibody wherein the immunoreactive region or site is obtained or derived from a first species and the constant region (which may be intact, partial or modified in accordance with the instant invention) is obtained from a second species. In preferred embodiments the target binding region or site will be from a non-human source (e.g. mouse) and the constant region is human. While the immunogenic specificity of the variable region is not generally affected by its source, a human constant region is less likely to elicit an immune response from a human subject than would the constant region from a non-human source.

C. Fusion Proteins

The starting polypeptides of the invention can also be a fusion protein which comprise at least an FcR binding portion of an Fc region. Preferably, the fusion proteins of the invention comprise a binding domain (which comprises at least one binding site). The subject fusion proteins may be bispecific (with one binding site for a first target and a second binding site for a second target) or may be multivalent (with two binding sites for the same target).

Exemplary fusion proteins reported in the literature include fusions of the T cell receptor (Gascoigne et al., Proc. Natl. Acad. Sci. USA 84:2936-2940 (1987)); CD4 (Capon et al., Nature 337:525-531 (1989); Traunecker et al., Nature 339:68-70 (1989); Zettmeissl et al., DNA Cell Biol. USA 9:347-353 (1990); and Byrn et al., Nature 344:667-670 (1990)); L-selectin (homing receptor) (Watson et al., J. Cell. Biol. 110:2221-2229 (1990); and Watson et al., Nature 349:164-167 (1991)); CD44 (Aruffo et al., Cell 61:1303-1313 (1990)); CD28 and B7 (Linsley et al., J. Exp. Med. 173:721-730 (1991)); CTLA-4 (Lisley et al., J. Exp. Med. 174:561-569 (1991)); CD22 (Stamenkovic et al., Cell 66:1133-1144 (1991)); TNF receptor (Ashkenazi et al., Proc. Natl. Acad. Sci. USA 88:10535-10539 (1991); Lesslauer et al., Eur. J. Immunol. 27:2883-2886 (1991); and Peppel et al., J. Exp. Med. 174:1483-1489 (1991)); and IgE receptor a (Ridgway and Gorman, J. Cell. Biol. Vol. 115, Abstract No. 1448 (1991)).

Ordinarily, the binding domain is fused C-terminally to the N-terminus of the Fc portion and in place of a cell anchoring region. For example, any transmembrane regions or lipid or phospholipids anchor recognition sequences of ligand binding receptor are preferably inactivated or deleted prior to fusion. DNA encoding the ligand or ligand binding partner is cleaved by a restriction enzyme at or proximal to the 5′ and 3′ends of the DNA encoding the desired ORF segment. The resultant DNA fragment is then readily inserted into DNA encoding a heavy chain constant region. The precise site at which the fusion is made may be selected empirically to optimize the secretion or binding characteristics of the soluble fusion protein. DNA encoding the fusion protein is then transfected into a host cell for expression.

In one embodiment, a fusion protein combines the binding domain(s) of the ligand or receptor (e.g. the extracellular domain (ECD) of a receptor) with at least one Fc portion and, optionally, a synthetic connecting peptide. In one embodiment, when preparing the fusion proteins of the present invention, nucleic acid encoding the binding domain of the ligand or receptor domain will be fused C-terminally to nucleic acid encoding the N-terminus of an Fc region. N-terminal fusions are also possible. Fusions may also be made to the C-terminus of the Fc portion of a constant domain, or immediately N-terminal to the CH1 of the heavy chain or the corresponding region of the light chain.

In one embodiment, the Fc region of the fusion protein includes substantially the entire Fc region of an antibody, beginning in the hinge region just upstream of the papain cleavage site which defines IgG Fc chemically (about residue 216 EU numbering, taking the first residue of heavy chain constant region to be 114) and ending at its C-terminus. The precise site at which the fusion is made is not critical; particular sites are well known and may be selected in order to optimize the biological activity, secretion, or binding characteristics of the molecule. Methods for making fusion proteins are known in the art.

For bispecific fusion proteins, the fusion proteins may be assembled as multimers, and particularly as heterodimers or heterotetramers. Generally, these assembled immunoglobulins will have known unit structures. A basic four chain structural unit is the form in which IgG, IgD, and IgE exist. A four chain unit is repeated in the higher molecular weight immunoglobulins; IgM generally exists as a pentamer of four basic units held together by disulfide bonds. IgA globulin, and occasionally IgG globulin, may also exist in multimeric form in serum. In the case of multimer, each of the four units may be the same or different.

Additonal exemplary ligands and their receptors that may be included in the subject fusion proteins include the following:

i) Cytokines and Cytokine Receptors

Cytokines have pleiotropic effects on the proliferation, differentiation, and functional activation of lymphocytes. Various cytokines, or receptor binding portions thereof, can be utilized in the fusion proteins of the invention. Exemplary cytokines include the interleukins (e.g. IL-1, IL-2, IL-3, IL-4, IL-5, IL-6, IL-7, IL-8, IL-10, IL-11, IL-12, IL-13, and IL-18), the colony stimulating factors (CSFs) (e.g. granulocyte CSF (G-CSF), granulocyte-macrophage CSF (GM-CSF), and monocyte macrophage CSF (M-CSF)), tumor necrosis factor (TNF) alpha and beta, and interferons such as interferon-α, β, or γ (U.S. Pat. Nos. 4,925,793 and 4,929,554).

Cytokine receptors typically consist of a ligand-specific alpha chain and a common beta chain. Exemplary cytokine receptors include those for GM-CSF, IL-3 (U.S. Pat. No. 5,639,605), IL-4 (U.S. Pat. No. 5,599,905), IL-5 (U.S. Pat. No. 5,453,491), IFNγ (EP0240975), and the TNF family of receptors (e.g., TNFα (e.g. TNFR-1 (EP 417, 563), TNFR-2 (EP 417,014) lymphotoxin beta receptor).

ii) Adhesion Proteins

Adhesion molecules are membrane-bound proteins that allow cells to interact with one another. Various adhesion proteins, including leukocyte homing receptors and cellular adhesion molecules, of receptor binding portions thereof, can be incorporated in a fusion protein of the invention. Leucocyte homing receptors are expressed on leucocyte cell surfaces during inflammation and include the β-1 integrins (e.g. VLA-1, 2, 3, 4, 5, and 6) which mediate binding to extracellular matrix components, and the β2-integrins (e.g. LFA-1, LPAM-1, CR3, and CR4) which bind cellular adhesion molecules (CAMs) on vascular endothelium. Exemplary CAMs include ICAM-1, ICAM-2, VCAM-1, and MAdCAM-1. Other CAMs include those of the selectin family including E-selectin, L-selectin, and P-selectin.

iii) Chemokines

Chemokines, chemotactic proteins which stimulate the migration of leucocytes towards a site of infection, can also be incorporated into a fusion protein of the invention. Exemplary chemokines include Macrophage inflammatory proteins (MIP-1-α and MIP-1-β), neutrophil chemotactic factor, and RANTES (regulated on activation normally T-cell expressed and secreted).

iv) Growth Factors and Growth Factor Receptors

Growth factors or their receptors (or receptor binding or ligand binding portions thereof) may be incorporated in the fusion proteins of the invention. Exemplary growth factors include Vascular Endothelial Growth Factor (VEGF) and its isoforms (U.S. Pat. No. 5,194,596); Fibroblastic Growth Factors (FGF), including aFGF and bFGF; atrial natriuretic factor (ANF); hepatic growth factors (HGFs; U.S. Pat. Nos. 5,227,158 and 6,099,841), neurotrophic factors such as bone-derived neurotrophic factor (BDNF), neurotrophin-3, -4, -5, or -6 (NT-3, NT-4, NT-5, or NT-6), or a nerve growth factor such as NGF-β platelet-derived growth factor (PDGF) (U.S. Pat. Nos. 4,889,919, 4,845,075, 5,910,574, and 5,877,016); transforming growth factors (TGF) such as TGF-alpha and TGF-beta (WO 90/14359), osteoinductive factors including bone morphogenetic protein (BMP); insulin-like growth factors-I and -II (IGF-I and IGF-II; U.S. Pat. Nos. 6,403,764 and 6,506,874); Erythropoietin (EPO); stem-cell factor (SCF), thrombopoietin (c-Mpl ligand), and the Wnt polypeptides (U.S. Pat. No. 6,159,462).

Exemplary growth factor receptors which may be used as targeting receptor domains of the invention include EGF receptors; VEGF receptors (e.g. Flt1 or Flk1/KDR), PDGF receptors (WO 90/14425); HGF receptors (U.S. Pat. Nos. 5,648,273, and 5,686,292), and neurotrophic receptors including the low affinity receptor (LNGFR), also termed as p75^{NTR} or p75, which binds NGF, BDNF, and NT-3, and high affinity receptors that are members of the trk family of the receptor tyrosine kinases (e.g. trkA, trkb (EP 455,460), trkC (EP 522,530)).

v) Hormones

Exemplary growth hormones for use as targeting agents in the fusion proteins of the invention include renin, human growth hormone (HGH; U.S. Pat. No. 5,834,598), N-methionyl human growth hormone; bovine growth hormone; growth hormone releasing factor; parathyroid hormone (PTH); thyroid stimulating hormone (TSH); thyroxine; proinsulin and insulin (U.S. Pat. Nos. 5,157,021 and 6,576,608); follicle stimulating hormone (FSH), calcitonin, luteinizing hormone (LH), leptin, glucagons; bombesin; somatropin; mullerian-inhibiting substance; relaxin and prorelaxin; gonadotropin-associated peptide; prolactin; placental lactogen; OB protein; or mullerian-inhibiting substance.

vi) Clotting Factors

Exemplary blood coagulation factors for use as targeting agents in the fusion proteins of the invention include the clotting factors (e.g., factors V, VII, VIII, X, IX, XI, XII and XIII, von Willebrand factor); tissue factor (U.S. Pat. Nos. 5,346,991, 5,349,991, 5,726,147, and 6,596,84); thrombin and prothrombin; fibrin and fibrinogen; plasmin and plasminogen; plasminogen activators, such as urokinase or human urine or tissue-type plasminogen activator (t-PA).

Other exemplary fusion proteins are taught, e.g., in WO0069913A1 and WO0040615A2. Another exemplary molecule that may be included in a fusion protein of the invention is IGSF9. Fusion proteins can be prepared using methods that are well known in the art (see for example U.S. Pat. Nos. 5,116,964 and 5,225,538).

III. Methods of Identifying Candidate Amino Acids For Modification

The present invention provides methods for identifying particular amino acid residues in the Fc region (or FcR binding portion thereof) of a starting Fc-containing polypeptide, that when altered by a mutation (e.g, by amino acid substitution), are predicted to result in the modulation of binding affinity to FcR and modulation of the effector function of the polypeptide.

The methods include molecular or computational modeling, which can be used to predict amino acid alterations in the Fc region to modulate (e.g., enhance or reduce) binding to an FcR. Generally, the methods begin with a “first” or “starting” polypeptide, or a complex (e.g. crystal strucuture or homology model) containing it, and result in a “second” or “altered” or “modified” polypeptide, which differs from the first polypeptide in that binding affinity to FcR is modulated and the modified polypeptide performs better in a particular therapeutic or diagnostic application. The modeling can be carried out in silico.

The methods may comprise one or more steps. For example, the method may comprise providing a structure of a complex, or data corresponding thereto, between the target Fc polypeptide and an FcR. In another or subsequent step, the methods may comprise identifying a defined residue or set of residues (ie. candidate amino acids) within the Fc region of a starting polypeptide that can be modified (e.g., mutated) and are predicted to affect the binding affinity of the polypeptide for FcR.

Preferred mutations that are introduced in the Fc region of a starting polypeptide include those mutations that alter an antigen-dependent effector function of the starting polypeptide (e.g. the ability of the polypeptide to mediate ADCC or complement fixation). In one embodiment, the mutation does not compromise any other existing effector functions of the starting polypeptide (e.g, antigen, ligand, or receptor binding or an Fc mediated effector function (other than FcR binding) or diminish from its intended use. Introduced mutations, therefore, preferably maintain many of the other advantages that the Fc region provides. For example, Fc-containing polypeptides often have ADCC functionality. This important cell killing activity would be partially or wholly lost in antibody constructs having truncated Fc regions. Maintaining Fc-dependent ADCC functionality can be important in certain applications because it can elicit a cell killing affect serving to enhance the efficacy of the anti-cancer drug or other drug that works by an ADCC dependent depletion mechanism.

In preferred embodiments, the altered polypeptides of the invention contain mutations that do not abolish, or more preferably, do not modulate, other desirable immune effector or receptor binding functions of the starting polypeptide. In particularly preferred embodiments, the altered polypeptides contain mutations that do not alter binding of the altered polypeptide to an Fc-binding protein that is capable of facilitating purification of the altered polypeptide, in particular Staphylococcal Protein A or G. The site on Fc responsible for binding to Protein A is known in the art (Deisenhofer J. 1981 Biochemistry. April 28;20(9):2361-70).

A. Sequence Based Analysis

In one embodiment, potential alternation sites are predicted based on a sequence comparison with the Fc region of the starting polypeptide and a mammalian Fc region with a dissimilar binding affinity for FcR. The sequences of the Fc regions are aligned and one or more corresponding amino acids from the sequence with dissimilar binding is substituted into the Fc region of the starting polypeptide.

In one embodiment, where reduced effector function is desired, a corresonding amino acid is chosen from an immunoglobulin of an unrelated mammalian species, wherein the immunoglobulin displays a lower affinity for the FcR. In an alternative embodiment, where higher effector function is desired, a homologous amino acid is chosen from an immunoglobulin of an unrelated mammalian species, wherein the immunoglobulin displays a higher affinity for the FcR.

B. Conformational Analysis

In another embodiment, the methods for identifying the target amino acid(s) comprise an analysis (e.g. visual inspection or computational analysis) of a starting polypeptide (e.g., an Fc-containing polypeptide) and/or a starting polypeptide bound to an Fc receptor (e.g., FcR).

The three-dimensional structure of a protein influences its biological activity and stability, and that structure can be determined or predicted in a number of ways. Generally, empirical methods use physical biochemical analysis. Alternatively, tertiary structure can be predicted using model building of three-dimensional structures of one or more homologous proteins (or protein complexes) that have a known three-dimensional structure. X-ray crystallography is perhaps the best-known way of determining protein structure (accordingly, the term “crystal structure” may be used in place of the term “structure”) (for example, the crystal structure of the human IgG1 Fc region has been determined (Disenhofer Biochemistry, (1981), 20: 2361-70), but estimates can also be made using circular dichroism, light scattering, or by measuring the absorption and emission of radiant energy. Other useful techniques include neutron diffraction and nuclear magnetic resonance (NMR). All of these methods are known to those of ordinary skill in the art, and they have been well described in standard textbooks (see, e.g., Physical Chemistry, 4th Ed., W.J. Moore, Prentiss-Hall, N.J., 1972, or Physical Biochemistry, K. E. Van Holde, Prentiss-Hall, N.J., 1971)) and numerous publications. Any of these techniques can be carried out to determine the structure of an Fc region, a polypeptide comprising an Fc region (or FcR binding portion thereof), or a complex of the polypeptide and FcR, which can then be analyzed according to predict amino acids for substitution and/or used to inform one or more steps of a procedure (e.g., such as those described herein).

Methods for forming crystals of an antibody, an antibody fragment, or scFv-antigen complex have been reported by, for example, van den Elsen et al. (Proc. Natl. Acad. Sci. USA 96:13679-13684, 1999, which is expressly incorporated by reference herein). Such art-recognized techniques can be carried out to determine the structure of a complex containing an Fc-containing polypeptide and FcR for analysis according to the methods of the present invention.

Alternatively, published structures of the complex, or data corresponding thereto, may be readily available from a commercial or public database, e.g. the Protein Data Bank. A number of structures have been solved of the extracellular domains of human FcγRs. For example, the co-crystal structure of the human IgG1 Fc fragment in complex with FcγRIIIB has been resolved at 3.2 Å (PDB accession code 1E4K; Sondermann et al., Nature, (2000), 406: 267-73). An additional X ray crystal structure of a human IgG1 Fc fragment in complex with FcγRIIIB has also recently been provided (PDB accession codes 1IIS and 1IIX;Radaev et al., J.Biol.Chem., (2001),276:16469-77). The structural coordinates (e.g. atomic coordinate) or 3D representations of these complexes can be obtained from the Protein Data Bank.

Where the structure of a complex (e.g. an X-ray structure) or data corresponding thereto is not known or available, a homology model using a related complex (e.g. from another species or a homologous ligand/receptor complex) may be utilized. For example, the crystal structure of the rat Fc-FcR complex can be used to model the interaction of human Fc with FcR.

Data corresponding to the Fc/ FcR complex can be evaluated to determine a potential alteration site. In another embodiment, the methods comprise an analysis (e.g. structural or computational analysis) of conformational differences between a free (ie. unbound) Fc-containing polypeptide and an Fc-containing polypeptide bound to FcR.

C. Electrostatic Optimization

The basic computational formulae used in carrying out the methods of the invention are provided in, e.g., U.S. Pat. No. 6,230,102, the contents of which are hereby incorporated by reference in the present application in their entirety. In one embodiment, polypeptides are altered (or “modified”) according to the results of a computational analysis of electrostatic forces between the polypeptide and FcR, preferably, in accordance to the discrete criteria or rules of the invention described herein. The computational analysis allows one to predict the optimal charge distribution within the polypeptide receptor complex, and one way to represent the charge distribution in a computer system is as a set of multipoles. Alternatively, the charge distribution can be represented by a set of point charges located at the positions of the atoms of the polypeptide. Once a charge distribution is determined (preferably, an optimal charge distribution), one can modify the polypeptide to match, or better match, that charge distribution.

The computational analysis can be mediated by a computer-implemented process that carries out the calculations described in U.S. Pat. No. 6,230,102 (or as described in Tidor and Lee, J. Chem. Phys. 106:8681, 1997; Kangas and Tidor, J. Chem. Phys. 109:7522, 1998). The computer program may be adapted to consider the real world context of polypeptide-FcR binding (and unlike other methods, this methods of the invention take into account, e.g., solvent, long-range electrostatics, and dielectric effects in the binding between a polypeptide and FcR in a solvent (e.g., an aqueous solvent such as water, phosphate-buffered saline (PBS), plasma, or blood)). The process is used to identify modifications to the polypeptide structure that will achieve a charge distribution on the modified polyeptide that minimizes the electrostatic contribution to binding free energy between the modified polypeptide and FcR (compared to that of the unmodified (“starting”) polypeptide. As is typical, the computer system (or device(s)) that performs the operations described here (and in more detail in U.S. Pat. No. 6,230,102) will include an output device that displays information to a user (e.g., a CRT display, an LCD, a printer, a communication device such as a modem, audio output, and the like). In addition, instructions for carrying out the method, in part or in whole, can be conferred to a medium suitable for use in an electronic device for carrying out the instructions. Thus, the methods of the invention are amendable to a high throughput approach comprising software (e.g., computer-readable instructions) and hardware (e.g., computers, robotics, and chips). The computer-implemented process is not limited to a particular computer platform, particular processor, or particular high-level programming language.

A useful process is set forth in U.S. Pat. No. 6,230,102 and a more detailed exposition is provided in Lee and Tidor (J. Chem. Phys. 106:8681-8690, 1997); each of which is expressly incorporated herein by reference.

The rules of the invention can be applied as follows. To modulate the FcR-binding affinity of a polypeptide, for example, to reduce, improve, or restore such binding, basic sequence and/or structural data is first acquired.

In one embodiment, the candidate amino acid residue(s) may be selected from those residues which are determined to have sub-optimal or optimal binding affinity. Alternatively or additionally, a target amino acid residue(s) may be may be selected from residues within the Fc region that are adjacent to the residue with optimal or sub-optimal binding affinity. Typically, an electrostatic charge optimization is first used to determine the position(s) of the Fc region that are sub-optimal for binding (Lee and Tidor, J. Chem. Phys. 106:8681-8690, 1997; Kangas and Tidor, J. Chem. Phys. 109:7522-7545, 1998).

Then, one or more mutations (i.e., modifications) is subjected to further computational analysis. Based on these calculations, the binding affinity is then determined for a subset of modified polypeptides having one or more modifications according to the rules of the invention.

Using a continuum electrostatics model, an electrostatic charge optimization can be performed on each side chain of the amino acids in the Fc of the polypeptide. A charge optimization gives charges at atom centers but does not always yield actual mutation(s). Accordingly, a round of charge optimizations can be performed with various constraints imposed to represent natural side chain characteristics at the positions of interest. For example, an optimization can be performed for a net side chain charge of −1, 0, and +1 with the additional constraint that no atom's charge exceeded a particular value, e.g., 0.85 electron charge units. Candidate amino acid side chain positions, and residue modifications at these positions, are then determined based on the potential gain in electrostatic binding free energy observed in the optimizations.

Binding free energy difference (in kcal/mol) in going from the native residue to a completely uncharged sidechain isostere, i.e., a residue with the same shape but no charges or partial charges on the atoms can be calculated. Negative numbers indicate a predicted increase of binding affinity.

In those instances in which binding free energy difference is favorable (ΔG<—0.25 kcal/mol) and associated with a transition from the native residue to a completely uncharged side chain isostere, i.e., a residue with the same shape but no charges or partial charges on the atoms, modifications from the set of amino acids with nonpolar sidechains, e.g., Ala, Cys, Ile, Leu, Met, Phe, Pro, Val are selected.

Where the binding free energy difference that can be obtained with an optimal charge distribution in the side chain and a net side chain charge of −1 is favorable (ΔG<—0.25 kcal/mol), modifications from the set of amino acids with negatively charged side chains, e.g., Asp, Glu are selected.

Similarly, where the binding free energy difference that can be obtained with an optimal charge distribution in the side chain and a net side chain charge of +1 is favorable (ΔG<—0.25 kcal/mol), modifications from the set of amino acids with positively charged sidechains, e.g., Arg, His, Lys are selected.

Finally, in those cases where the binding free energy difference that can be obtained with an optimal charge distribution in the side chain and a net side chain charge of 0 is favorable (ΔG<—0.25 kcal/mol), modifications from the set of amino acids with uncharged polar sidechains, e.g., Asn, Cys, Gln, Gly, His, Met, Phe, Ser, Thr, Trp, Tyr, to which are added Cys, Gly, Met and Phe are selected.

As described herein, the designed modified polypeptides can be built in silico and the binding energy recalculated. Modified side chains can be built by performing a rotamer dihedral scan in CHARMM, using dihedral angle increments of 60 degrees, to determine the most desirable position for each side chain. Binding energies are then calculated for the wild type (starting) and mutant (modified) complexes using the Poisson-Boltzmann electrostatic energy and additional terms for the van der Waals energy and buried surface area.

Results from these computational modification calculations are then reevaluated as needed, for example, after subsequent reiterations of the method either in silico or informed by additional experimental structural/functional data. The rules allow for several predictions to be made which can be categorized as follows:
1) modifications at the interaction interface involving residues on the polypeptide that become partially buried upon binding FcR (interactions are improved by making hydrogen bonds);
2) modifications of polar residues on the polypeptide that become buried upon binding and thus pay a desolvation penalty but do not make any direct electrostatic interactions with the receptor (improvements are usually made by modifying to a hydrophobic residue with similar shape to the wild-type residue or by adding a residue that can make favorable electrostatic interactions); and
3) modifications of surface residues on the polypeptide that are in regions of uncomplementary potentials. These modifications are believed to improve long-range electrostatic interactions between the polypeptide and FcR without perturbing packing interactions at the binding interface.

Thus practiced, the rules of the invention allow for the successful prediction of affinity altering, (e.g., reducing or enhancing), side chain modifications. These findings can be classified into three general classes of modifications. The first type of modification involves residues at the interface across from a charged group on the antigen capable of making a hydrogen bond; the second type involves buried polar residues that pay a desolvation penalty upon binding but do not make back electrostatic interactions; and the third type involves long-range electrostatic interactions.

The first type of modification is determined by inspection of basic physical/chemical considerations, as these residues essentially make hydrogen bonds with unsatisfied hydrogen partners of the antigen. Unlike other methods, the rules of the invention allowed for surprising residue modifications in which the cost of desolvation is allowed to outweigh the beneficial interaction energy.

The second type of modification represents still another set of modifications, as the energy gained is primarily a result of eliminating an unfavorable desolvation while maintaining non-polar interactions.

The third type of modification concerns long-range interactions that show potential for significant gain in affinity. These types of modifications are particularly interesting because they do not make direct contacts with the antigen and, therefore, pose less of a perturbation in the delicate interactions at the polypeptide-FcR interface.

Accordingly, when the desired side chain chemistries are determined for the candidate amino acid position(s) according to the rules, the residue position(s) is then modified or altered, e.g., by substitution, insertion, or deletion, as further described herein.

In addition to the above rules for polypeptide modification, it is noted that certain determinations, e.g., solvent effects can be factored into initial (and subsequent) calculations of optimal charge distributions.

A charge optimization results in a set of optimal charges at atom centers but does not yield actual mutation suggestions. Once a charge optimization is determined using the methods recited above, one or more of the target amino acid residues, or any adjacent amino acid residues in the polypeptide (e.g., residues in or around the CH₂ domain or the FcR binding loop of the Fc region) can be altered (e.g. mutated) based on the results of the charge optimization. In this process the optimal charge distribution is analyzed and mutations are selected that are closer to optimal than the current residue. For example, amino acid substitutions may be selected that are a match for, a better match for, or are closer to optimal than the current residue. One, or more than one, mutation may be selected such that the optimal charge distribution is achieved. The preferred mutation may be selected by visual inspection of the data or by computation analysis of the data.

Presently, the software used to examine electrostatic forces models an optimal charge distribution and the user then determines what amino acid substitution(s) or alteration(s) would improve that distribution. Accordingly, such steps (e.g., examining the modeled, optimal charge distribution and determining a sequence modification to improve antigen binding) are, or can be, part of the methods now claimed. However, as it would not be difficult to modify the software so that the program includes the selection of amino acid substitutions (or alterations), in the future, one may need only examine that output and execute the suggested change (or some variation of it, if desired).

In another embodiment, amino acids are grouped into the following three groups (1) non-polar amino acids that have uncharged side chains (e.g. A, L, I, V, G, P). These amino acids are usually implicated in hydrophobic interactions;
(2) amino acids having polar amino acids that have net zero charge, but have non-zero partial charges in different portions of their side chains (e.g. M, F, W, S, Y, N, Q, C). These amino acids can participate in hydrophobic interactions and electrostatic interactions.
(3) charged amino acids that can have non-zero net charge on their side chains (e.g. R, K, H, E, D). These amino acids can participate in hydrophobic interactions and electrostatic interactions.

In one embodiment, at least one mutation altering the affinity of polypeptide-Fc interaction is a mutation from one of the following three categories:
(1) mutations that change the charge distribution of the at the interaction interface or in the regions of uncomplimentary electrostatic potentials between FcR and polypeptide away from the interface. These changes can include substitutions between the groups on polar, non-polar, and charged amino acids (they will always change the location of partial charges), as well as substitutions within the group of polar aminoacids and within the group of charged amino acids as long as they alter the charge distribution (for instance C has a partial negative charge on SG atom and partially positive on HG atom. Whereas N has a partial positive charge on SG, and HD atoms, and partial negative charge on ND and OD atoms; hence, substitution of C for N will ater charge distribution). For example, in one embodiment, a substitution of an amino acid that is non-polar (with zero charges at all atoms in a sidechain) with an amino acid that is polar (with a zero net charge, but having partial charges on atoms in a sidechain) or visa versa;
(2) mutations of polar or charged residues on the antibody that become buried upon binding, and thus pay a desolvation penalty (energetic cost of removal of solvent upon binding) but do not make any favorable electrostatic interactions with the FcR. In this case improvements are made by mutation to non-polar amino acids that do not interact with solvent and, therefore, will not pay a desolvation penalty upon binding.
(3) mutations of surface residues that change the shape of the molecule, thus affecting the dielectric properties of the medium between polypeptide and FcR. Since solvent has higher screening capacity (dielectric constant) than a protein, charges will interact stronger through protein than through solvent. Therefore, filling (or clearing) the space between charges on polypeptide and FcR with protein side sidechains will modulate their interaction. These mutations include amino acid substitutions where substituent has a different shape of a sidechain than an original amino acid (all chages except for ones between isosteres: V to T, D to N, N to D, L to D, L to N, D to L, N to L, Q to E, and E to Q). For substitution with the group on non-polar amino acids, this phenomenon would be the only effect on electrostatic interaction between polypeptide and FcR.

In one embodiment, an amino acid of the starting polypeptide which is uncharged substituted with a charged amino acid. In another embodiment, an uncharged amino acid of the starting polypeptide is substituted with another uncharged amino acid. In another embodiment, an amino acid of the starting polypeptide (e.g., an uncharged or negatively charged amino acid) is substituted with a positively charged amino acid. Positively charged amino acids include histidine, lysine, and asparagine. In another embodiment, an amino acid of the starting polypeptide (e.g., an uncharged or positively charged amino acid) is substituted with a negatively charged amino acid. Negatively charged amino acids include aspartate (aspartic acid) and glutamate (glutamic acid). In certain embodiments, when introduced in the altered polypeptide, the amino acid which is substituted changes the charge of the polypeptide such that the altered polypeptide has a different net charge than the starting polypeptide.

D. Side Chain Repacking

In another embodiment, the method for selecting a preferred amino acid substitution comprises the application of sidechain repacking techniques to a structure (e.g. the crystal structure) of a complex containing the Fc-containing polypeptide and the FcR. In a sidechain repacking calculation, the target residues can be modified computationally, and the stability of the resulting Fc polypeptide mutants in the conformation bound to the FcR's evaluated computationally. The sidechain repacking calculation generates a ranked list of the variants that have altered stability (i.e., altered intramolecular energy).

In another embodiment, the method for selecting a preferred amino acid substitution comprises the application of sidechain repacking techniques to a structure (e.g. a crystal structure) of a complex containing two polypeptides (e.g. an Fc-containing polypeptide and an FcR. Mutants which result in a desired alteration (e.g. increase or decrease) of receptor binding affinity can then be selected for experimental expression.

In one embodiment, the target residues are close to regions in the Fc molecule that display conformational changes between the receptor-bound and free structure. For example, target residues may be within about 5-25 Å of such regions (e.g., residues within about 5, 10, 15, 20, or 25 Å of such regions). These residues, or any subset of them, are allowed to mutate to any of the 20 naturally occurring amino acid residues.

The number of protein mutants that is evaluated computationally can be very large, since every variable amino acid position can be mutated into all 20 standard amino acids. Exemplary computational algorithms used to rank the results of the computational analysis include dead-end elimination and tree search algorithms (see for example, Lasters et al. (Protein Eng. 8:815-822, 1995), Looger and Hellinga (J. Mol. Biol. 307:429-445, 2001), and Dahiyat and Mayo (Protein Sci. 5:895-903, 1996)).

In an exemplary embodiment, the region or feature displaying a conformational difference is the CH2-CH3 interface. Typically, the CH2-CH3 interface displays a widening of the angle between domains CH2 and CH3 upon transition from a first “closed” conformation in the free or unbound state, to a second “open” conformation upon binding to an Fc receptor (e.g. an Fc gamma receptor).

In one embodiment, target amino acid residues include residues in the CH2-CH3 interface of the Fc region whose local molecular environment changes between the closed and open forms. Such target residues can be mutated such that they will not fit in closed (ie. unbound Fc) conformation but do fit in the open (ie. bound Fc) conformation. For example, the inventors identified atarget amino acids at EU positions 376 because it facilitates such a conformational transition. In addition, the inventors identified the amino acid at position 378 (in CH3) as a target residue because substitution of A378 with a charged residue or residue of sufficient steric bulk will favor the open conformation due to steric interactions with residues P247 and P248 (both in the CH2 domain) in the closed conformation.

In another embodiment, target amino acid residues include residues in the CH2-CH3 interface of the Fc region that exhibit steric crowding in the open conformation and therefore disfavor opening of the conformation. Such target residues can be mutated such that a steric barrier to opening of the CH2-CH3 interface is removed. For example, the inventors identified the amino acid at EU positions 251 and 435 because residue L251 (in CH2) moves closer to H435 in the open conformation.

In another exemplary embodiment, the region or feature displaying a conformational difference, is the fucose saccharide residue within the N-linked glycan attached to N297 of the Fc region, as well as residues in the vicinity (e.g. <10 Å) of the fucose residue (“fticose interacting residues”). Although the cause of the effect is unknown, it is known in the art that removal of the fucose residue results in a significant descrease the affinity of an Fc region for an Fc receptor (e.g. CD l 6) (see Shields et al., J. Biol. Chem., (2002), 277: 26733-40). The inventors have concluded that the fucose residue is forced into an energetically unfavorable state as the Fc binds to an Fc receptor (e.g. CD16). The nature of this unfavorable state could be either enthalpic in nature, entropic in nature, or a combination of both enthalpic and entropic effects. One of the causes of the unfavorable enthalpic state could be that the fucose gets pushed towards the fucose interacting residues as the Fc binds to the Fc receptor, resulting in steric repulsion. Alternatively, steric crowding of the fucose interacting residues could result in an unfavorable entropic effect because the fucose is conformationally constrained.

Accordingly, in one embodiment, target amino acid residues include flicose interacting residues that cause favorable or unfavorable effecuts upon by movement of the fucose residue upon binding of the Fc region to an Fc receptor. Such target residues can be mutated to reduce or increase the enthalpic and/or entropic cost paid by the fucose upon binding. For example, the inventors identified a candidate amino acids at EU positions 294, 296, and 301.

In one embodiment, the sidechain repacking calculation is used to identify mutations that make the open (bound) form of Fc energetically more favorable than the closed (free) form. In another embodiment, the sidechain repacking calculation is used to identify mutations that make the closed (free) form of Fc energetically less favorable than the open (bound) form.

In a more specific embodiment, the sidechain repacking calculation is used to identify mutations which result in a higher stability (ie. lower calculated intramolecular free energy) for the open (bound) form than the closed (free) form. In another specific embodiment, the sidechain repacking calculation is used to identify mutations which result in a lower stability (i.e. higher calculated intramolecular free energy) for the closed (free) form than the open (bound) form. Fc polypeptide variants with a higher stability in the receptor-bound conformation are expected to have a higher affinity for an Fc receptor.

In another embodiment, the sidechain repacking calculation is used to identify mutations that make the closed (free) form of Fc energetically more favorable than the open (closed) form. In another embodiment, the sidechain repacking calculation is used to identify mutations that make the open (bound) form of Fc energetically less favorable than the closed (free) form. In a more specific embodiment, the sidechain repacking calculation is used to identify mutations which result in a higher stability (ie. lower calculated intramolecular free energy) for the closed (free) form than the open (bound) form. In another specific embodiment, the sidechain repacking calculation is used to identify mutations which result in a lower stability (i.e. higher calculated intramolecular free energy) for the open (bound) form than the closed (free) form. Fc polypeptide variants with a higher stability in the closed (free) form are expected to have a lower affinity for an Fc receptor.

Mutants can be selected for modulated binding to Fc gamma receptor based on the propensity of the altered polypeptide to favor or disfavor an “open” or “bound” conformation (i.e. a conformation that is bound to an FcγR. Alternatively, mutations can be selected that favor or disfavors a “closed” or “unbound” (e.g. a conformation that is not bound to an FcR).

E. 3-D Visualization

In one embodiment, since the bound form of Fc has a widened angle between the CH2 and CH3 domains, a visual analysis (e.g. using a 3-D molecular visualizer) of a predicted mutation can be visually analysed to predict mutations that will favor or disfavor a particular molecular conformation.

In one embodiment the mutation results in an increase in affinity of an Fc-containing polypeptide for an Fc receptor. In one exemplary embodiment, a preferred amino acid substitution is a substitution which favors an “open”, Fc receptor-bound, conformation (e.g. a conformation bound to FcR). In another exemplary embodiment, the preferred amino acid substitution disfavors a “closed” or unbound conformation (e.g. a conformation not bound to FcR).

In another embodiment, the mutation results in a decrease in affinity of an Fc-containing polypeptide for an Fc receptor. In one exemplary embodiment, a preferred amino acid substitution is a substitution which favors a “closed” or unbound conformation. In another exemplary embodiment, the preferred amino acid substitution disfavors an “open” or bound conformation (e.g. a conformation bound to FcR).

In another embodiment, the mutation results in an amino acid at the target site that does not fit in the closed conformation but does fit in the open conformation, for example, due to steric crowding. Exemplary amino acid substitutions include amino acids with bulkier side chains. Exemplary amino acid having side chain chemistry of sufficient steric bulk include tyrosine, tryptophan, arginine, lysine, histidine, glutamic acid, glutamine, and methionine, or analogs or mimetics thereof. For example, the inventors predicted that the following mutations at amino acid residues D376 and A378 (both in the CH3 domain) would strongly disfavor the open conformation: D376F, D376H, D376K, D376R, D376W, D376Y, A378F, A378H, A378K, A378Q, A378R, A378W, and A378Y.

In another specific embodiment, the mutation results in an amino acid at the target site that facilitate a conformational transition to an “open” conformation, for example, due to removal of a steric barrier. Exemplary amino acid substitutions include amino acids with smaller sized side chains, including glycine, alanine, valine, serine, aspartate, and glutamate.

For example, the inventors predicted that the following mutations at amino acid residues L251 (in CH2) and H435 (in CH3) would favor the open conformation: L251A, L251S, L251G, H435A, H435G, and H435S.

In another specific embodiment, the mutation reduces the entropic and/or enthalpic cost paid by the fucose residue upon binding by reducing the size of the chain chain of a fucose interaction residue. For example, the inventors predicted the following mutations at amino acid residues Q294, Y296, or R301 (all in CH2 domain) would favor the open conformation: Q294G, Q294A, Q294S, Q294T, Q294N, Y296G, Y296A, Y296S, Y296N, R301G, R301A, R301K, R301N, R301Q, R301S, or R301T.

F. Further Optimization of FcR Binding Affinity

An altered polypeptide generated by the methods of the invention can be re-modeled and further altered to further modulate FcR binding (e.g., to further enhance or further decrease binding). Thus, the steps described above can be followed by additional steps, including, e.g.,: (a) obtaining data corresponding to the structure of a complex between the altered or “second” polypeptide and the receptor; (b) determining, using the data (which we may refer to as “additional data” to distinguish it from the data obtained and used in the first “round”), a representation of an additional charge distribution with the constant region of the second polypeptide that minimizes electrostatic contribution to binding free energy between the second polypeptide and the receptor; and (c) expressing a third polypeptide that binds to the receptor, the third polypeptide having a sequence that differs from that of the second polypeptide by at least one amino acid residue. In addition, empirical binding data can be used to inform further optimization. Yet additional rounds of optimization can be carried out.

IV. Methods of Altering Polypeptides

Having arrived at a desired mutation to make in a starting polypeptide one can use any of a variety of available methods to produce an altered polypeptide comprising the mutation. Such polypeptides can, for example, be produced by recombinant methods. Moreover, because of the degeneracy of the genetic code, a variety of nucleic acid sequences can be used to encode each desired polypeptide.

Exemplary art recognized methods for making a nucleic acid molecule encoding an amino acid sequence variant of a starting polypeptide include, but are not limited to, preparation by site-directed (or oligonucleotide-mediated) mutagenesis, PCR mutagenesis, and cassette mutagenesis of an earlier prepared DNA encoding the polypeptide.

Site-directed mutagenesis is a preferred method for preparing substitution variants. This technique is well known in the art (see, e.g., Carter et al. Nucleic Acids Res. 13:4431-4443 (1985) and Kunkel et al., Proc. Natl. Acad. Sci. USA 82:488 (1987)). Briefly, in carrying out site-directed mutagenesis of DNA, the parent DNA is altered by first hybridizing an oligonucleotide encoding the desired mutation to a single strand of such parent DNA. After hybridization, a DNA polymerase is used to synthesize an entire second strand, using the hybridized oligonucleotide as a primer, and using the single strand of the parent DNA as a template. Thus, the oligonucleotide encoding the desired mutation is incorporated in the resulting double-stranded DNA.

PCR mutagenesis is also suitable for making amino acid sequence variants of the starting polypeptide. See Higuchi, in PCR Protocols, pp.177-183 (Academic Press, 1990); and Vallette et al., Nuc. Acids Res. 17:723-733 (1989). Briefly, when small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template.

Another method for preparing variants, cassette mutagenesis, is based on the technique described by Wells et al., Gene 34:315-323 (1985). The starting material is the plasmid (or other vector) comprising the starting polypeptide DNA to be mutated. The codon(s) in the parent DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the starting polypeptide DNA. The plasmid DNA is cut at these sites to linearize it. A double-stranded oligonucleotide encoding the sequence of the DNA between the restriction sites but containing the desired mutation(s) is synthesized using standard procedures, wherein the two strands of the oligonucleotide are synthesized separately and then hybridized together using standard techniques. This double-stranded oligonucleotide is referred to as the cassette. This cassette is designed to have 5′ and 3′ ends that are compatible with the ends of the linearized plasmid, such that it can be directly ligated to the plasmid. This plasmid now contains the mutated DNA sequence.

Alternatively, or additionally, the desired amino acid sequence encoding a polypeptide variant can be determined, and a nucleic acid sequence encoding such amino acid sequence variant can be generated synthetically.

It will be understood by one of ordinary skill in the art that the polypeptides of the invention having altered FcR binding may further be modified such that they vary in amino acid sequence, but not in desired activity. For example, additional nucleotide substitutions leading to amino acid substitutions at “non-essential” amino acid residues may be made to the protein For example, a nonessential amino acid residue in an immunoglobulin polypeptide may be replaced with another amino acid residue from the same side chain family. In another embodiment, a string of amino acids can be replaced with a structurally similar string that differs in order and/or composition of side chain family members, i.e., a conservative substitutions, in which an amino acid residue is replaced with an amino acid residue having a similar side chain, may be made.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Aside from amino acid substitutions, the present invention contemplates other modifications of the starting Fc region amino acid sequence in order to generate an Fc region variant with altered effector function. One may, for example, delete one or more amino acid residues of the Fc region in order to reduce or enhance binding to an FcR. In one embodiment, one or more of the Fc region residues can be modified in order to generate such an Fc region variant. Generally, no more than one to about ten Fc region residues will be deleted according to this embodiment of the invention. The Fc region herein comprising one or more amino acid deletions will preferably retain at least about 80%, and preferably at least about 90%, and most preferably at least about 95%, of the starting Fc region or of a native sequence human Fc region.

One may also make amino acid insertion Fc region variants, which variants have altered effector function. For example, one may introduce at least one amino acid residue (e.g. one to two amino acid residues and generally no more than ten residues) adjacent to one or more of the Fc region positions identified herein as impacting FcR binding. By “adjacent” is meant within one to two amino acid residues of a Fc region residue identified herein. Such Fc region variants may display enhanced or diminished FcR binding.

Such Fc region variants will generally comprise at least one amino acid modification in the Fc region. In one embodiment amino acid modifications may be combined. For example, the variant Fc region may include two, three, four, five, etc substitutions therein, e.g. of the specific Fc region positions identified herein. In another embodiment, an altered polypeptide may have altered binding to FcR and to another Fc receptor.

The Fc region consists of two identical protein chains. Accordingly, in one embodiment, the mutations are applied to both protein chains. In another embodiment, the mutations are applied only in one protein chain.

V. Preferred Alterations

Altered polypeptides of the invention contain at least one mutation (e.g. an amino acid substitution) within their Fc region. In one embodiment, the substituted amino acid(s) are located within the CH2 domain of the Fc region. In another embodiment, the substituted amino acid(s) are located within the CH3 domain of the Fc region. In another embodiment, substituted amino acids are located within both the CH2 and CH3 domain of the Fc region.

In one embodiment, an altered polypeptide of the invention comprises at least one amino acid mutation in the Fc region that serve to enhance the effector function of the molecule. Molecules with enhanced effector function are useful, e.g., when clearance of a target molecule or cell to which it is bound is desired.

In another embodiment, an altered polypeptide of the invention comprises at least one amino acid mutation in the Fc region that serves to decrease effector function of the molecule. Molecules with decreased effector function are less likely to cause release of immune mediators, which can be undesirable under certain circumstances.

Alteration in antigen-dependent effector functions may be predicted from a difference between the starting antibody and the altered antibody with respect to their FcR binding affinity.

In some embodiments, the altered polypeptides of the invention will exhibit altered antigen-dependent effector functions without altering antigen-independent effector functions (e.g. half-life). In other embodiments, the altered polypeptides will alteration in both antigen-independent effector function and antigen-dependent effector functions. In one embodiment, one or more the mutations disclosed herein may confer increased antigen-dependent effector function and another mutation may confer decreased half-life.

In another embodiment, one or more the mutations disclosed herein may confer increased antigen-dependent effector function and another mutation may confer increased half-life. In another embodiment, one or more the mutations disclosed herein may confer decreased antigen-dependent effector function and another mutation may confer decreased half-life. In another embodiment, one or more the mutations disclosed herein may confer decreased antigen-dependent effector function and another mutation may confer increased half-life.

In one embodiment, effector function is reduced by reducing the affinity of binding to an Fc receptor (FcR), such as FcγRI, FcγRIIa, FcγRIIIa, and/or FcγRIIIb or increasing binding to FcγRIIb. In one embodiment, effector function is increased by increasing the affinity of binding to an Fc receptor (FcR), such as FcγRI, FcγRIIa, FcγRIIIa, and/or FcγRIIIb or decreasing binding to FcγRIIb.

In another embodiment, effector function is reduced by reducing binding to a complement protein, such as C1q. In another embodiment, effector function is increased by increasing binding to a complement protein, such as C1q.

In a related embodiment, binding is modulated (e.g., increased or decreased) by a factor of about 1-fold to about 15-fold or more.

In a particular embodiment, the altered polypeptide comprises a substitution at an amino acid position corresponding to an EU position selected from the group consisting of 234, 236, 239, 241, 251, 265, 268, 270, 292, 293, 294, 296, 298, 299, 301, 326, 328, 330, 332, 333, 334, 376, 378, and 435.

In another embodiment, an altered polypeptide comprises at least an FcγR binding portion of an Fc region wherein the polypeptide comprises at least one mutation (up to all) compared to a starting polypeptide and wherein the at least one mutation is selected from the group consisting of:

a substitution at EU amino acid position 234 with aspartic acid or glutamine;

a substitution at EU amino acid position 236;

a substitution at EU amino acid position 239 with proline;

a substitution at EU amino acid position 241 with glutamine or histidine;

a substitution at EU amino acid position 251 with alanine, serine, or glycine;

a substitution at EU amino acid position 265 with a negatively charged amino acid;

a substitution at EU amino acid position 268 with proline or negatively charged amino acid;

a substitution at EU amino acid position 270 with glutamic acid;

a substitution at EU amino acid position 293 with aspartic acid;

a substitution at EU amino acid position 294 with serine, threonine, or asparagine;

a substitution at EU amino acid position 296 with alanine, histadine, asparagine, serine, threonine, or phenylalanine;

a substitution at EU amino acid position 298 with asparagine;

a substitution at EU amino acid position 301 with alanine, lysine, threonine, asparagine, glutamine or serine;

a substitution at EU amino acid position 326 with aspartic acid, glutamic acid, asparagine, or glutamine;

a substitution at EU amino acid position 328 with threonine, lysine, aspartic acid, glutamic acid, asparagine, or glutamine;

a substitution at EU amino acid position 330 with histidine;

a substitution at EU amino acid position 332 with histidine, aspartic acid, glutamic acid, asparagine, or glutamine;

a substitution at EU amino acid position 334 with aspartic acid, glutamic acid, asparagine, or glutamine;

a substitution at EU amino acid position 376 with histidine, lysine, arginine, tryptophan, or tyrosine or with an amino acid of suffieicent steric bulk or a charged amino acid.

In another embodiment, the altered polypeptide can include any one or any combination (and up to all) of the following mutations:

a substitution at EU position 234 with aspartate or glutamine; a substitution at EU position 236 with alanine; a substitution at EU position 239 with aspartate, histidine, proline, or glutamate; a substitution at EU position 241 with glutamine or histidine; a substitution at EU position 251 with alanine, serine, or glycine; a substitution at EU position 265 with glutamate; a substitution at EU position 268 with proline or aspartate; a substitution at EU position 270 with glutamate; a substitution at EU position 292 with alanine; a substitution at EU position 293 with aspartate; a substitution at EU position 294 with alanine, serine, threonine, or asparagine; a substitution at EU position 296 with alanine, serine, threonine, asparagine, glutamine, histidine, or phenylalanine; a substitution at EU position 298 with alanine or asparagine; a substitution at EU position 299 with cysteine; a substitution at EU position 301 with alanine, lysine, asparagine, glutamine, serine, or threonine; a substitution at EU position 326 with aspartate, glutamate, asparagine, or glutamine; a substitution at EU position at 328 with asparagine, threonine, aspartate, glutamate, or glutamine; a substitution at EU position 330 with leucine or histidine; a substitution at EU position 332 with aspartate, glutamate, glutamine, or histidine; a substitution at EU position 333 with aspartate; a substitution at EU position 334 with asparagine, aspartate, glutamate, or glutamine; a substitution at EU position 338 with methionine; a substitution at EU position 376 with arginine, lysine, histidine, phenylalanine, or tryptophan; a substitution at EU position 378 with lysine, glutamine, arginine, histidine, phenylalanine, tyrosine, or tryptophan; or a substitution at EU position 435 with alanine, serine, or glycine.

In another embodiment, the substitution is introduced in the Fc region of IgG1 and is selected from one of the following mutations: L234D, L234Q, G236A, S239D, S239E, S239P, S239H, F241Q, F241H, L251A, L251S, L251G, D265E, H268P, H268D, D270E, R292A, E293D, Q294A, Q294S, Q294T, Q294N, E294A, E294S, E294T, E294N, Y296A, Y296S, Y296N, Y296Q, Y296T, Y296H, Y296F, S298A, S298N, T299C, R301A, R301K, R301N, R301Q, R301S, R301T, K326D, K326E, K326N, K326Q, L328T, L328N, L328D, L328Q, L328E, A330H, A330L, I332D, I332Q, I332E, I332H, E333D, K334N, K334D, K334Q, K334E, K334V, K334R, K338M, N376R, N376K, N376H, N376F, N376W, D376R, D376K, D376H, D376y, D376W, A378K, A378Q, A378R, A378H, A378F, A378Y, A378W, H435A, H435S, or H435G.

In exemplary embodiment, the altered polypeptide can include combinations (e.g. two, three, or four) of any of the following mutations: S239D, S239E, L261A, S298A, A330L, I332D, I332E, A378F, A378K, A378W, A378Y, H435G, or H435S.

Particularly preferred double mutants include S239E/I332D, S239E/I332E, S239D/I332D, S239D/I332E, S239D/A378F, S239D/A378K, S239D/A378F, S239D/A378W, S239D/A378Y, S239D/A378G, S239D/A378S, I332D/A378F, I332D/A378K, I332D/A378W, I332D/A378Y, I332D/H435G, I332D/H435S, and I332D/L261 A.

In one embodiment, an amino acid mutation is made at at least one amino acid position selected from the group consisting of:

an amino acid from amino acid position 234 to 241, inclusive (close to FcgR interface); from amino acid position 247 to 252, inclusive (close to CH2-CH3 interface in CH2); from amino acid position 265 to 270, inclusive (close to FcgR interface); from amino acid position 292 to 301, inclusive (close to FcgR interface); from amino acid position 326 to 334, inclusive (close to FcgR interface); from amino acid position 373 to 380, inclusive (close to CH2-CH3 interface in CH3); and from amino acid position 428 to 435, inclusive (close to CH2-CH3 interface in CH3)

Polypeptides of the invention may further contain one or more amino acid mutations which are known in the art to alter effector function. In preferred embodiments, the polypeptide contains one or more amino acid mutations that impart a desired antigen-independent effector function (e.g. longer half life). In another embodiment, a polypeptide of the invention contains one or more amino acid mutations that impart a desired antigen-dependent effector function that complements (e.g., in an additive or synergistic manner) a mutation described herein.

Accordingly, in one embodiment, a polypeptide may be mutation adjacent to, or close to, sites in the hinge link region (e.g., at residues 234-9 according to the EU numbering as in Kabat), order to alter binding to an FcR. In another embodiment, a polypeptide may contain a mutations in the N-terminus of the CH2 or CH3 domains. In another embodiment, C1q binding properties can be altered by additionally mutating at least one of the amino acid residues 318, 320, and 322 of the F region. It is also known that mutations in the glycosylation site at residue 297 can abrogate or reduce many effector functions, e.g. CDCC activity. Accordingly, a polypeptide of the invention may additionally comprise such a mutation.

As set forth above it will be understood that the subject compositions may comprise one or more of the mutations set forth herein. In one embodiment, the altered polypeptides of the invention comprise only one of the mutations listed herein. In one embodiment, the altered polypeptides of the invention comprise only two of the mutations listed herein. In one embodiment, the altered polypeptides of the invention comprise only three of the mutations listed herein. In one embodiment, the altered polypeptides of the invention comprise only four of the mutations listed herein.

A. Altered Polypeptides with Enhanced FcR Binding Affinity

In one embodiment, the present invention provides altered Fc polypeptides with an enhanced affinity for an Fc gamma receptor or Fc binding protein as compared to their corresponding target polypeptides. In preferred embodiments, the altered Fc polypeptides of the invention have enhanced affinity for activating Fc receptors (e.g. CD64, CD32a/c, or CD16).

In one embodiment, the altered Fc polypeptides have an enhanced affinity for an Fc gamma receptor III (e.g. CD16a) as compared to their corresponding target polypeptides.

In one embodiment, altered Fc polypeptide with enhanced FcγRIII binding affinity may comprise at least one amino acid substitution at one of the following EU positions: 239, 261, 268, 298, 330, 332, 334,376, 378, and 435.

In one exemplary embodiment, the altered Fc polypeptide with enhanced FcγRIII binding affinity comprises an Fc region of an IgG1 molecule. Preferably the Fc region contains at least one of the following mutations: S239D, S239E, L261A, H268D, S298A, A330H, A330L, I332D, I332E, I332Q, K334V, A378F, A378K, A378W, A378Y, H435S, or H435G. More preferably, the Fc region contains at least one of the following mutations: S239D, S239E, I332D or I332E or H268D. Still more preferably, the Fc region contains at least one of the following mutations: I332D or I332E or H268D.

In another exemplary embodment, the Fc region contains at least one of the following double mutations: S239E/I332D, S239E/I332E, S239D/I332D, S239D/I332E, S239D/A378F, S239D/A378K, S239D/A378F, S239D/A378W, S239D/A378Y, S239D/A378G, S239D/A378S, I332D/A378F, I332D/A378W, or I332D/A378Y. More preferably, the Fc region contains at least one of the following double mutations: S239E/I332D, S239E/I332E, S239D/I332D, or S239D/I332E. In another embodiment, an altered polyeptide of the invention comprises at least one of the following double mutations: S239E/H268D, I332D/ H268D, I332E/ H268D.

In one embodiment, the altered Fc polypeptides have an enhanced affinity for a complement protein (e.g. C1q) as compared to their corresponding target polypeptides.

In one embodiment, altered Fc polypeptide with enhanced complement binding affinity may comprise at least one amino acid substitution at one of the following EU positions: 251, 326, 334, 378, or 435.

In one exemplary embodiment, the altered Fc polypeptide with enhanced complement binding affinity comprises an Fc region of an IgG1 molecule. Preferably the Fc region contains at least one of the following mutations: L251A, L251 G, K326D, K334R, A378F, A378K, A378W, A378Y, H435G, or H435S. More preferably, the Fc region contains at least one of the following mutations: A378F, A378W, or A378Y.

In preferred embodiments of the present invention, the binding affinity of the altered polypeptide is enhanced by at least about 30%, 50%, 80%, 2-fold, 3-fold, 4-fold, 5-fold, 10-fold, 15-fold, 20-fold, 25-fold, 30-fold, 40-fold, 50-fold, 60-fold, 70-fold, 80-fold, 90-fold, or 100-fold.

Polypeptides with enhanced effector function may be of particular value for administration to a patient when destruction of a target cell to which the binding portion of a polypeptide of the invention binds is desired, e.g., in the case of a patient with a tumor cell.

B. Altered Polypeptides with Reduced Binding Affinity

In one embodiment, the present invention provides altered Fc polypeptides with reduced binding affinity for an Fc gamma receptor or Fc binding protein as compared to their corresponding target polypeptides.

In one embodiment, the altered Fc polypeptides have a reduced affinity for Fc gamma receptor III (e.g. CD 16a) as compared to their corresponding target polypeptides.

In one embodiment, altered Fc polypeptide with reduced FcγRIII binding affinity may comprise at least one arnino acid substitution at one of the following EU positions: 234, 236, 239, 241, 251, 261, 265, 268, 293, 294, 296, 298, 301, 328, 332, 338, 376, 378, or 435.

In one exemplary embodiment, the altered Fc polypeptide with reduced FcltlII binding affinity comprises an Fc region of an IgG1 molecule. Preferably the Fc region comprises at least one of the following mutations: L234D, L234Q, G236A, S239H, S239P, F241H, F241Q, L251G, L261A, D265E, H268P, E293D, E294N, E294S, E294T, Y296A, Y296F, Y296H, Y296Q, Y296S, Y296T, S298N, R301A, R301K, R301N, R301Q, L328D, L328E, L328T, L328N, L328Q, L328K, I332H, I332K, K338M, D376H, D376K, D376R, D376W, A378H, H435A, H435G, or H435S. More preferably, the Fc region comprises at least one of the following mutations: S239H, S239P, L251G, D265E, E294S, Y296H, Y296S, Y296T, S298N, R301Q, L328D, L328E, D376K, or D376W. Still more preferably, the Fc region comprises at least one of the following mutations: S239H, S239P, L251 G, D265E, Y296S, Y296T, or L328D.

In one embodiment, an altered polypeptide of the invention binds C1q to a lesser degree than a starting polypeptide and comprises at least one mutation selected from the group consisting of: L328K or I332K.

In another exemplary embodment, the Fc region comprises the following double mutations: I332D/L261A and L328K/I332K

In another embodiment, the altered Fc polypeptides have reduced affinity for a Fc gamma receptor II (e.g. CD32b) as compared to their corresponding target polypeptides. In one embodiment, the altered Fc polypeptide with reduced FcγRII binding affinity may comprise an amino acid substitution at EU position 328. In an exemplary embodiment, the altered Fc polypeptide with reduced FcγRII binding affinity comprises an Fc region of an IgG1 molecule. Preferably the Fc region comprises the following mutation: L328N.

In another embodiment, the altered Fc polypeptides have reduced affinity for a Fc gamma receptor I (e.g. CD64) as compared to their corresponding target polypeptides. In one embodiment, the altered Fc polypeptide with reduced FcγRI binding affinity may comprise an amino acid substitution at EU position 328 or 334. In an exemplary embodiment, the altered Fc polypeptide with reduced FcγRI binding affinity comprises an Fc region of an IgG1 molecule. Preferably the Fc region comprises one of the following mutations: L328E or K334R.

In another embodiment, the altered Fc polypeptides have a reduced binding affinity for a complement protein (e.g. C1q) as compared to the starting polypeptide.

In one embodiment, altered Fc polypeptide with reduced complement binding affinity may comprise at least one amino acid substitution at one of the following EU positions: 239, 294, 296, 301, 328, 332, 333, or 376.

In one exemplary embodiment, the altered Fc polypeptide with reduced complement binding affinity comprises an Fc region of an IgG1 molecule. Preferably the Fc region contains at least one of the following mutations: S239D, S239E, E294A, E294N, Y296A, Y296H, Y296Q, Y296S, Y296T, R301N, L328D, L328E, L328N, L328K, L328Q, I332K, E333D, or D376W. More preferably, the Fc region contains at least one of the following mutations: L328D, L328E, L328N, L328Q, L328K or D376W.

In a preferred embodiment of the present invention, the binding affinity of the altered polypeptide is reduced by at least about 30%, 40%, 50%, 60%, 70%, 80%, 85%, 90%, 95%, 97%, 98%, or 99% as compared to a starting polypeptide. In one embodiment, at least one effector function of the molecule (e.g., ADCC or complement activation) is reduced to a corresponding degree.

Polypeptides with reduced effector function may be particularly desirable in situations in which the destruction of cells to which the binding portion of a polypeptide of the invention binds is not desired.

V. Expression of Altered Polypeptides

The polypeptides of the invention, e.g., starting polypeptides and modified polypeptides may be produced by recombinant methods.

For example, a polynucleotide sequence encoding a polypeptide can be inserted in a suitable expression vector for recombinant expression. Where the polypeptide is an antibody, polynucleotides encoding additional light and heavy chain variable regions, optionally linked to constant regions, may be inserted into the same or different expression vector. An affinity tag sequence (e.g. a His(6) tag) may optionally be attached or included within the starting polypeptide sequence to facilitate downstream purification. The DNA segments encoding immunoglobulin chains are the operably linked to control sequences in the expression vector(s) that ensure the expression of immunoglobulin polypeptides. Expression control sequences include, but are not limited to, promoters (e.g., naturally-associated or heterologous promoters), signal sequences, enhancer elements, and transcription termination sequences. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the polypeptide.

These expression vectors are typically replicable in the host organisms either as episomes or as an integral part of the host chromosomal DNA. Commonly, expression vectors contain selection markers (e.g., ampicillin-resistance, hygromycin-resistance, tetracycline resistance or neomycin resistance) to permit detection of those cells transformed with the desired DNA sequences (see, e.g., U.S. Pat. No. 4,704,362).

E. coli is one prokaryotic host particularly useful for cloning the polynucleotides (e.g., DNA sequences) of the present invention. Other microbial hosts suitable for use include bacilli, such as Bacillus subtilus, and other enterobacteriaceae, such as Salmonella, Serratia, and various Pseudomonas species.

Other microbes, such as yeast, are also useful for expression. Saccharomyces and Pichia are exemplary yeast hosts, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for methanol, maltose, and galactose utilization.

In addition to microorganisms, mammalian tissue culture may also be used to express and produce the polypeptides of the present invention (e.g., polynucleotides encoding immunoglobulins or fragments thereof). See Winnacker, From Genes to Clones, VCH Publishers, N.Y., N.Y. (1987). Eukaryotic cells are actually preferred, because a number of suitable host cell lines capable of secreting heterologous proteins (e.g., intact immunoglobulins) have been developed in the art, and include CHO cell lines, various Cos cell lines, HeLa cells, 293 cells, myeloma cell lines, transformed B-cells, and hybridomas. Expression vectors for these cells can include expression control sequences, such as an origin of replication, a promoter, and an enhancer (Queen et al., Immunol. Rev. 89:49 (1986)), and necessary processing information sites, such as ribosome binding sites, RNA splice sites, polyadenylation sites, and transcriptional terminator sequences. Preferred expression control sequences are promoters derived from immunoglobulin genes, SV40, adenovirus, bovine papilloma virus, cytomegalovirus and the like. See Co et al., J. Immunol. 148:1149 (1992).

The vectors containing the polynucleotide sequences of interest (e.g., the heavy and light chain encoding sequences and expression control sequences) can be transferred into the host cell by well-known methods, which vary depending on the type of cellular host. For example, calcium chloride transfection is commonly utilized for prokaryotic cells, whereas calcium phosphate treatment, electroporation, lipofection, biolistics or viral-based transfection may be used for other cellular hosts. (See generally Sambrook et al., Molecular Cloning: A Laboratory Manual (Cold Spring Harbor Press, 2nd ed., 1989). Other methods used to transform mammalian cells include the use of polybrene, protoplast fuision, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

The subject polypeptide can also be incorporated in transgenes for introduction into the genome of a transgenic animal and subsequent expression, e.g., in the milk of a transgenic animal (see, e.g., Deboer et al. U.S. Pat. No. 5,741,957; Rosen U.S. Pat. No. 5,304,489; and Meade U.S. Pat. No. 5,849,992. Suitable transgenes include coding sequences for light and/or heavy chains in operable linkage with a promoter and enhancer from a mammary gland specific gene, sugh as casein or beta lactoglobulin.

Altered polypeptides (e.g., polypeptides) can be expressed using a single vector or two vectors. For example, antibody heav y and light chains may be cloned on separate expression vectors and co-transfected into cells.

In one embodiment, signal sequences may be used to facilitate expression of polypeptides of the invention.

Once expressed, the polypeptides can be purified according to standard procedures of the art, including ammonium sulfate precipitation, affinity columns (e.g., protein A or protein G), column chromatography, HPLC purification, gel electrophoresis and the like (see generally Scopes, Protein Purification (Springer-Verlag, N.Y., (1982)). In a preferred embodiment, the purification procedure may employ the use of a multimeric Fc receptor of the invention as described below.

VI. Analysis of Binding Affinity

Binding affinity can be measured in a variety of ways. Generally, and regardless of the precise manner in which affinity is defined or measured, the methods of the invention modulate binding affinity to FcR when they generate a polypeptide that is superior in any aspect of its clinical application to the starting polypeptide from which it was made (for example, the methods of the invention are considered effective or successful when a modified polypeptide, e.g., has a better clinical outcome than the starting polypeptide, can be administered at a lower dose or less frequently or by a more convenient route of administration or has reduced side effects.

An alteration in the effector function of a polypeptide can be determined by measuring its binding affinity for a particular Fc receptor. In one embodiment, an alteration of antigen-dependent effector function can be determined by measuring the binding affinity of the altered polypeptide for an Fc gamma receptor.

An alteration in the binding affinity of an altered polypeptide of the invention may be determined by comparing the binding affinity of the altered polypeptide with a suitable control polypeptide (e.g. the corresponding starting polypeptide). In one embodiment, an alteration of binding affinity may be determined by comparing the binding affinity of the altered polypeptide in first assay with the binding affinity of the control polypeptide in a second binding assay. In alternative embodiments, an alteration of binding affinity may be determined by comparing the binding affinity of the altered polypeptide and the control polypeptide in the same assay. For example, the assay may be performed as a competitive binding assay where the binding affinity of the altered polypeptide is evaluated with increasing concentrations of the control polypeptide.

i) Cell-free Assays

Several in vitro, cell-free assays for testing the effector functions (e.g. FcR binding affinity) of altered polypeptides have been described in the art. Preferably, the cell-based assay is capable of evaluating binding of altered antibodies to soluble forms of Fc receptors. Automation and HTS technologies may be utilized in the screening procedures. Screening may employ the use of labels (e.g. isotopic labels, chromophores, fluorophore, lumiphores, or epitopes) that enable detection. The labels may be attached to the Fc receptor or the Fc-containing polypeptide that is assayed.

Exemplary cell-free assays include, but are not limited to, FRET (fluorescence resonance energy transfer), BRET (bioluminescence resonance energy transfer), Alphascreen (Amplied Luminescent Proximity Homogeneous)-based assays, scintillation proximity assays, ELISA (enzyme-linked immunosorbent assays), SPR (surface plasmon resonance, such as BIACORE®), isothermal titration calorimetry, differential scanning calorimetry, gel electrophoresis, analytical ultracentrifugation, and chromatography, including gel-filtration chromatography.

ii) Cell-based Assays

Several in vitro, cell-based assays for testing the effector functions (e.g. FcR binding affinity) of altered polypeptides have been described in the art. Preferably, the cell-based assay is capable of evaluating binding of altered antibodies to surface forms of the Fc receptors. Exemplary cell-based assays include bridging assays and flow cytometry.

In an exemplary embodiment, the FcR binding affinity of an altered antibody can be measured using an FcR bridging assay. FcR (e.g. FcN or FcγR) binding affinities can be measured with assays based on the ability of the antibody to form a “bridge” between antigen and a FcR bearing cell.

iii) Model Animal Assays

The altered polypeptides of the invention may also be administered to a model animal to test its potential for use in therapy, either for veterinary purposes or as an animal model of human disease, e.g., an immune disease or condition stated above, e.g., by testing the effector function of the antibody. Regarding the latter, such animal models may be useful for evaluating the therapeutic efficacy of antibodies of the invention (e.g., testing of effector function, dosages, and time courses of administration).

Examples of animal models which can be used for evaluating the therapeutic efficacy of altered polypeptides of the invention for preventing or treating tumor formation include tumor xenograft models.

Examples of animal models which can be used for evaluating the therapeutic efficacy of altered polypeptides of the invention for preventing or treating rheumatoid arthritis (RA) include adjuvant-induced RA, collagen-induced RA, and collagen mAb-induced RA (Holmdahl et al., Immunol. Rev. 184:184, 2001; Holmdahl et al., Ageing Res. Rev. 1:135, 2002; Van den Berg, Curr. Rheumatol. Rep. 4:232, 2002).

Examples of animal models which can be used for evaluating the therapeutic efficacy of altered polypeptides of the invention for preventing or treating inflammatory bowel disease (IBD) include TNBS-induced IBD, DSS-induced IBD, and (Padol et al., Eur. J. Gastrolenterol. Hepatol. 12:257, 2000; Murthy et al., Dig. Dis. Sci. 38:1722, 1993).

Examples of animal models which can be used for evaluating the therapeutic efficacy of altered polypeptides of the invention for preventing or treating glomerulonephritis include anti-GBM-induced glomerulonephritis (Wada et al., Kidney Int. 49:761-767, 1996) and anti-thyl -induced glomerulonephritis (Schneider et al., Kidney Int. 56:135-144, 1999).

Examples of animal models which can be used for evaluating the therapeutic efficacy of antibodies or antigen-binding fragments of the invention for preventing or treating multiple sclerosis include experimental autoimmune encephalomyelitis (EAE) (Link and Xiao, Immunol. Rev. 184:117-128,2001).

VIII. Further Modification of Altered Fc-containing Polypeptides

Altered Fc-containing polypeptide may be further modified to provide a desired effect. For example, in certain embodiments, the altered polypeptides may be modified (e.g. by chemical or genetic means) by conjugated (ie. physically linked) to an additional moiety to an additional moiety, i.e., a functional moiety such as, for example, a PEGylation moiety, a blocking moiety, a detectable moiety, a diagnostic moiety, and/or a therapeutic moiety, that serves to improve the desired function (e.g. therapeutic efficacy) of the polypeptide. Chemical conjugation may be performed by randomly or by site-specific modification of particular residues within the altered polypeptide. Exemplary functional moieties are first described below followed by useful chemistries for linking such functional moieties to different amino acid side chain chemistries of an altered polypeptide.

a) Functional Moieties

Examples of useful functional moieties include, but are not limited to, a PEGylation moiety, a blocking moiety, detectable moiety, a diagnostic moiety, and a therapeutic moiety.

Exemplary PEGylation moieties include moieties of polyalkylene glycol moiety, for example, a PEG moiety and preferably a PEG-maleimide moiety. Preferred pegylation moieties (or related polymers) can be, for example, polyethylene glycol (“PEG”), polypropylene glycol (“PPG”), polyoxyethylated glycerol (“POG”) and other polyoxyethylated polyols, polyvinyl alcohol (“PVA) and other polyalkylene oxides, polyoxyethylated sorbitol, or polyoxyethylated glucose. The polymer can be a homopolymer, a random or block copolymer, a terpolymer based on the monomers listed above, straight chain or branched, substituted or unsubstituted as long as it has at least one active sulfone moiety. The polymeric portion can be of any length or molecular weight but these characteristics can affect the biological properties. Polymer average molecular weights particularly useful for decreasing clearance rates in pharmaceutical applications are in the range of 2,000 to 35,000 daltons. In addition, if two groups are linked to the polymer, one at each end, the length of the polymer can impact upon the effective distance, and other spatial relationships, between the two groups. Thus, one skilled in the art can vary the length of the polymer to optimize or confer the desired biological activity. PEG is useful in biological applications for several reasons. PEG typically is clear, colorless, odorless, soluble in water, stable to heat, inert to many chemical agents, does not hydrolyze, and is nontoxic.

Preferably PEGylation moieties are attached to altered Fc-containing polypeptides of the invention that have enhanced-life. A PEGylation moiety can serve to further enhance the half-life of the altered polypeptide by increasing the molecule's apparent molecular weight. The increased apparent molecular weight reduces the rate of clearance from the body following subcutaneous or systemic administration. In many cases, a PEGylation also serve to decrease antigenicity and immunogenicity. In addition, PEGylation can increase the solubility of the altered polypeptide.

Exemplary blocking moieties include include cysteine adducts, cystine, mixed disulfide adducts, or other compounds of sufficient steric bulk and/or charge such that antigen-dependent effector function is reduced, for example, by inhibiting the ability of the Fc region to bind an Fc receptor or complement protein. Preferably, said blocking moieties are conjugated to altered polypeptides of the invention with reduced effector function such that effector function is further reduced.

Exemplary detectable moieties which may be useful for conjugation to the altered polypeptides of the invention include fluorescent moieties, radioisotopic moieties, radiopaque moieties, and the like, e.g. detectable labels such as biotin, fluorophores, chromophores, spin resonance probes, or radiolabels. Exemplary fluorophores include fluorescent dyes (e.g. fluorescein, rhodamine, and the like) and other luminescent molecules (e.g. luminal). A fluorophore may be environmentally-sensitive such that its fluorescence changes if it is located close to one or more residues in the modified protein that undergo structural changes upon binding a substrate (e.g. dansyl probes). Exemplary radiolabels include small molecules containing atoms with one or more low sensitivity nuclei (¹³C, ¹⁵N, ²H, ¹²⁵I, ¹²³I, 99Tc, ⁴³K, ⁵²Fe, ⁶⁷Ga, ⁶⁸Ga, ¹¹¹In and the like). Other useful moieties are known in the art.

Examples of diagnostic moieties which may be useful for conjugation to the altered polypeptides of the invention include detectable moieties suitable for revealing the presence of a disease or disorder. Typically a diagnostic moiety allows for determining the presence, absence, or level of a molecule, for example, a target peptide, protein, or proteins, that is associated with a disease or disorder. Such diagnostics are also suitable for prognosing and/or diagnosing a disease or disorder and its progression.

Examples of therapeutic moieties which may be useful for conjugation to the altered polypeptides of the invention include, for example, anti-inflammatory agents, anti-cancer agents, anti-neurodegenerative agents, and anti-infective agents. The functional moiety may also have one or more of the above-mentioned functions.

Exemplary therapeutics include radionuclides with high-energy ionizing radiation that are capable of causing multiple strand breaks in nuclear DNA, and therefore suitable for inducing cell death (e.g., of a cancer). Exemplary high-energy radionuclides include: ⁹⁰Y, ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re. These isotopes typically produce high energy α- or β-particles which have a short path length. Such radionuclides kill cells to which they are in close proximity, for example neoplastic cells to which the conjugate has attached or has entered. They have little or no effect on non-localized cells and are essentially non-immunogenic.

Exemplary therapeutics also include cytotoxic agents such as cytostatics (e.g. alkylating agents, DNA synthesis inhibitors, DNA-intercalators or cross-linkers, or DNA-RNA transcription regulators), enzyme inhibitors, gene regulators, cytotoxic nucleosides, tubulin binding agents, hormones and hormone antagonists, anti-angiogenesis agents, and the like.

Exemplary therapeutics also include alkylating agents such as the anthracycline family of drugs (e.g. adriamycin, carminomycin, cyclosporin-A, chloroquine, methopterin, mithramycin, porfiromycin, streptonigrin, porfiromycin, anthracenediones, and aziridines). In another embodiment, the chemotherapeutic moiety is a cytostatic agent such as a DNA synthesis inhibitor. Examples of DNA synthesis inhibitors include, but are not limited to, methotrexate and dichloromethotrexate, 3-amino-1,2,4-benzotriazine 1,4-dioxide, aminopterin, cytosine β-D-arabinofuranoside, 5-fluoro-5′-deoxyuridine, 5-fluorouracil, ganciclovir, hydroxyurea, actinomycin-D, and mitomycin C. Exemplary DNA-intercalators or cross-linkers include, but are not limited to, bleomycin, carboplatin, carmustine, chlorambucil, cyclophosphamide, cis-diammineplatinum(II) dichloride (cisplatin), melphalan, mitoxantrone, and oxaliplatin.

Exemplary therapeutics also include transcription regulators such as actinomycin D, daunorubicin, doxorubicin, homoharringtonine, and idarubicin. Other exemplary cytostatic agents that are compatible with the present invention include ansamycin benzoquinones, quinonoid derivatives (e.g. quinolones, genistein, bactacyclin), busulfan, ifosfamide, mechlorethamine, triaziquone, diaziquone, carbazilquinone, indoloquinone EO9, diaziridinyl-benzoquinone methyl DZQ, triethylenephosphoramide, and nitrosourea compounds (e.g. carmustine, lomustine, semustine).

Exemplary therapeutics also include cytotoxic nucleosides such as, for example, adenosine arabinoside, cytarabine, cytosine arabinoside, 5-fluorouracil, fludarabine, floxuridine, ftorafur, and 6-mercaptopurine; tubulin binding agents such as taxoids (e.g. paclitaxel, docetaxel, taxane), nocodazole, rhizoxin, dolastatins (e.g. Dolastatin-10, -11, or -15), colchicine and colchicinoids (e.g. ZD6126), combretastatins (e.g. Combretastatin A-4, AVE-6032), and vinca alkaloids (e.g. vinblastine, vincristine, vindesine, and vinorelbine (navelbine)); anti-angiogenesis compounds such as Angiostatin KI-3, DL-α-difluoromethyl-ornithine, endostatin, fumagillin, genistein, minocycline, staurosporine, and (±)-thalidomide.

Exemplary therapeutics also include hormones and hormone antagonists, such as corticosteroids (e.g. prednisone), progestins (e.g. hydroxyprogesterone or medroprogesterone), estrogens, (e.g. diethylstilbestrol), antiestrogens (e.g. tamoxifen), androgens (e.g. testosterone), aromatase inhibitors (e.g. aminogluthetimide), 17-(allylamino)-17-demethoxygeldanamycin, 4-amino-1,8-naphthalimide, apigenin, brefeldin A, cimetidine, dichloromethylene-diphosphonic acid, leuprolide (leuprorelin), luteinizing hormone-releasing hormone, pifithrin-α, rapamycin, sex hormone-binding globulin, and thapsigargin.

Exemplary therapeutics also include enzyme inhibitors such as, S(+)-camptothecin, curcumin, (−)-deguelin, 5,6-dichlorobenz-imidazole 1-β-D-ribofuranoside, etoposide, formestane, fostriecin, hispidin, 2-imino-1-imidazolidineacetic acid (cyclocreatine), mevinolin, trichostatin A, tyrphostin AG 34, and tyrphostin AG 879.

Exemplary therapeutics also include gene regulators such as 5-aza-2′-deoxycytidine, 5-azacytidine, cholecalciferol (vitamin D₃), 4-hydroxytamoxifen, melatonin, mifepristone, raloxifene, trans-retinal (vitamin A aldehydes), retinoic acid, vitamin A acid, 9-cis-retinoic acid, 13-cis-retinoic acid, retinol (vitamin A), tamoxifen, and troglitazone.

Exemplary therapeutics also include cytotoxic agents such as, for example, the pteridine family of drugs, diynenes, and the podophyllotoxins. Particularly useful members of those classes include, for example, methopterin, podophyllotoxin, or podophyllotoxin derivatives such as etoposide or etoposide phosphate, leurosidine, vindesine, leurosine and the like.

Still other cytotoxins that are compatible with the teachings herein include auristatins (e.g. auristatin E and monomethylauristan E), calicheamicin, gramicidin D, maytansanoids (e.g. maytansine), neocarzinostatin, topotecan, taxanes, cytochalasin B, ethidium bromide, emetine, tenoposide, colchicin, dihydroxy anthracindione, mitoxantrone, procaine, tetracaine, lidocaine, propranolol, puromycin, and analogs or homologs thereof.

Other types of functional moieties are known in the art and can be readily used in the methods and compositions of the present invention based on the teachings contained herein.

b) Chemistries for Linking Functional Moieties to Amino Acid Side Chains

Chemistries for linking the foregoing functional moieties be they small molecules, nucleic acids, polymers, peptides, proteins, chemotherapeutics, or other types of molecules to particular amino acid side chains are known in the art (for a detailed review of specific linkers see, for example, Hermanson, G. T., Bioconjugate Techniques, Academic Press (1996)).

Exemplary art recognized linking groups for sulflhydryl moieties (e.g., cysteine, or thiol side chain chemistries) include, but are not limited to, activated acyl groups (e.g., alpha-haloacetates, chloroacetic acid, or chloroacetamide), activated alkyl groups, Michael acceptors such as maleimide or acrylic groups, groups which react with sulfhydryl moieties via redox reactions, and activated di-sulfide groups. The sulfhydryl moieties may also be linked by reaction with bromotrifluoroacetone, alpha-bromo-beta-(5-imidazoyl)propionic acid, chloroacetyl phosphate, N-alkylmaleimides, 3-nitro-2-pyridyl disulfide, methyl-2-pyridyl disulfide, p-chloromercuribenzoate, 2-chloromercuri-4-nitrophenol, or chloro-7-nitrobenzo-2-oxa-1,3-diazole.

In a preferred embodiment, a cysteine or other amino acid with thiol side chain chemistry is linked during.or subsequent to the production of an Fc containing polypeptide. For example, when producing the modified Fc containing polypeptide using cell culture, conditions are provided such that a free cysteine in solution can form a cysteine adduct with the thiol side chain of the Fc containing polypeptide. The so formed adduct may be used to inhibit glycosylation and/or effector function, or, subsequently subjected to reducing conditions to remove the adduct and thereby allow for the use of one of the aforementioned sulfhydryl chemistries.

Exemplary art recognized linking groups for hydroxyl moieties (e.g., serine, threonine, or tyrosine side chain chemistries) include those described above for sulfflydryl moieties including activated acyl groups, activated alkyl groups, and Michael acceptors.

Exemplary art recognized linking groups for amine moieties (e.g., asparagine or arginine side chain chemistries) include, but are not limited to, N-succinimidyl, N-sulfosuccinimidyl, N-phthalimidyl, N-sulfophthalimidyl, 2-nitrophenyl, 4-nitrophenyl, 2,4-dinitrophenyl, 3-sulfonyl-4-nitrophenyl, 3-carboxy-4-nitrophenyl, imidoesters (e.g., methyl picolinimidate), pyridoxal phosphate, pyridoxal, chloroborohydride, trinitrobenzenesulfonic acid, O-methyliosurea, and 2,4-pentanedione.

Exemplary art recognized linking groups for acidic moieties (e.g., aspartic acid or glutamic side chain chemistries) include activated esters and activated carbonyls. Acidic moieties can also be selectively modified by reaction with carbodiimides (R′N—C—N—R′) such as 1-cyclohexyl-3-[2-morpholinyl-(4-ethyl)]carbodiimide or 1-ethyl-3-(4-azonia-4,4-dimethylpentyl)carbodiimide.

Where the functional moiety desired is a PEGylation moiety, PEGylation reactions which are well known in the art may be employed. For example, in one method, the PEGylation is carried out via an acylation reaction or an alkylation reaction with a reactive polyethylene glycol molecule (or an analogous reactive water-soluble polymer). A water-soluble polymer for pegylation of the antibodies and antibody fragments of the invention is polyethylene glycol (PEG). In another embodiment, the polymer for pegylation is polyethylene glycol-maleimide (i.e., PEG-maleimide).

Methods for preparing pegylated antibodies and antibody fragments of the invention will generally comprise the steps of a) reacting the antibody or antibody fragment with polyethylene glycol, such as a reactive ester or aldehyde derivative of PEG, under conditions whereby the antibody or antibody fragment becomes attached to one or more PEG groups, and b) obtaining the reaction products. It will be apparent to one of ordinary skill in the art to select the optimal reaction conditions or the acylation reactions based on known parameters and the desired result. In one embodiment, a particular amino acid reside can be targeted, for example, the first amino acid residue altered in order to inhibit glycosylation of a second amino acid residue, and preferably where the first amino acid is a cysteine or has a thiol chemistry.

IX. Prophylactic, Diagnostic, and Therapeutic Methods

The present invention has general utility when the altered polypeptide (e.g., an antibody or fusion protein) binds a cell-surface antigen, where the binding provokes a required effector response. One example of an effector-mediated response is the reduction in the root cause of a disorder (e.g., elimination of tumor cells or of antigen-bearing cells that are involved in immune or inflammatory responses). In another embodiment, one or more symptom(s) of a disorder can be reduced. In another embodiment, the compositions described herein can be used to alter an effector-mediated response in a diagnostic reagent (e.g., an antibody used for imaging tumors).

A. Anti-Tumor Therapy

Accordingly, in certain embodiments, the altered polypeptides of the present invention are useful in the prevention or treatment of cancer. In one embodiment, an altered polypeptide blocks autocrine or paracrine growth (e.g., by binding to a receptor without transducing a signal, or by binding to a growth factor). In preferred embodiments, the altered polypeptide is capable of binding to a tumor-associated antigen.

In one embodiment, the altered polypeptides may reduce tumor size, inhibit tumor growth and/or prolong the survival time of tumor-bearing animals. In general, the disclosed invention may be used to prophylactically or therapeutically treat any neoplasm comprising an antigenic marker that allows for the targeting of the cancerous cells by the modified antibody. Exemplary cancers or neoplasias that may be prevented or treated include, but are not limited to bladder cancer, breast cancer, head and neck cancer, prostate cancer, colo-rectal cancer, melanoma or skin cancer, breast cancer, ovarian cancer, cervical cancer, endometrial cancer, kidney cancer, lung cancer (e.g. small cell and non-squamos cell cancers), pancreatic cancer, and multiple myeloma. More particularly, the modified antibodies of the instant invention may be used to treat Kaposi's sarcoma, CNS neoplasias (capillary hemangioblastomas, meningiomas and cerebral metastases), melanoma, gastrointestinal and renal sarcomas, rhabdomyosarcoma, glioblastoma (preferably glioblastoma multiforme), leiomyosarcoma, retinoblastoma, papillary cystadenocarcinoma of the ovary, Wilm's tumor or small cell lung carcinoma. It will be appreciated that appropriate starting polypeptides may be derived for tumor associated antigens related to each of the forgoing neoplasias without undue experimentation in view of the instant disclosure.

Exemplary hematologic malignancies that are amenable to treatment with the disclosed invention include Hodgkins and non-Hodgkins lymphoma as well as leukemias, including ALL-L3 (Burkitt's type leukemia), chronic lymphocytic leukemia (CLL) and monocytic cell leukemias. It will be appreciated that the altered polypeptides and methods of the present invention are particularly effective in treating a variety of B-cell lymphomas, including low grade/follicular non-Hodgkin's lymphoma (NHL), cell lymphoma (FCC), mantle cell lymphoma (MCL), diffuse large cell lymphoma (DLCL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL and Waldenstrom's Macroglobulinemia. It should be clear to those of skill in the art that these lymphomas will often have different names due to changing systems of classification, and that patients having lymphomas classified under different names may also benefit from the combined therapeutic regimens of the present invention. In addition to the aforementioned neoplastic disorders, it will be appreciated that the disclosed invention may advantageously be used to treat additional malignancies bearing compatible tumor associated antigens.

B. Immune Disorder Therapies

Besides neoplastic disorders, the altered polypeptides of the instant invention are particularly effective in the treatment of autoimmune disorders or abnormal immune responses. In this regard, it will be appreciated that the altered polypeptide of the present invention may be used to control, suppress, modulate or eliminate unwanted immune responses to both external antigens and autoantigens. For example, in one embodiment, the antigen is an autoantigen. In another embodiment, the antigen is an allergan. In yet other embodiments, the antigen is an alloantigen or xenoantigen. Use of the disclosed modified polypeptides to reduce an immune response to alloantigens and xenoantigens is of particular use in transplantation, for example to inhibit rejection by a transplant recipient of a donor graft, e.g. a tissue or organ graft or bone marrow transplant. Additionally, suppression or elimination of donor T cells within a bone marrow graft is useful for inhibiting graft versus host disease.

In yet other embodiments the altered polypeptides of the present invention may be used to treat immune disorders that include, but are not limited to, allergic bronchopulmonary aspergillosis; Allergic rhinitis Autoimmune hemolytic anemia; Acanthosis nigricans; Allergic contact dermatitis; Addison's disease; Atopic dermatitis; Alopecia areata; Alopecia universalis; Amyloidosis; Anaphylactoid purpura; Anaphylactoid reaction; Aplastic anemia; Angioedema, hereditary; Angioedema, idiopathic; Ankylosing spondylitis; Arteritis, cranial; Arteritis, giant cell; Arteritis, Takayasu's; Arteritis, temporal; Asthma; Ataxia-telangiectasia; Autoimmune oophoritis; Autoimmune orchitis; Autoimmune polyendocrine failure; Behcet's disease; Berger's disease; Buerger's disease; bronchitis; Bullous pemphigus; Candidiasis, chronic mucocutaneous; Caplan's syndrome; Post-myocardial infarction syndrome; Post-pericardiotomy syndrome; Carditis; Celiac sprue; Chagas's disease; Chediak-Higashi syndrome; Churg-Strauss disease; Cirrhosis; Cogan's syndrome; Cold agglutinin disease; CREST syndrome; Crohn's disease; Cryoglobulinemia; Cryptogenic fibrosing alveolitis; Dermatitis herpetifomis; Dermatomyositis; Diabetes mellitus; Diamond-Blackfan syndrome; DiGeorge syndrome; Discoid lupus erythematosus; Eosinophilic fasciitis; Episcleritis; Drythema elevatum diutinum; Erythema marginatum; Erythema multiforme; Erythema nodosum; Familial Mediterranean fever; Felty's syndrome; Fibrosis pulmonary; Glomerulonephritis, anaphylactoid; Glomerulonephritis, autoimmune; Glomerulonephritis, post-streptococcal; Glomerulonephritis, post-transplantation; Glomerulopathy, membranous; Goodpasture's syndrome; Granulocytopenia, immune-mediated; Granuloma annulare; Granulomatosis, allergic; Granulomatous myositis; Grave's disease; Hashimoto's thyroiditis; Hemolytic disease of the newborn; Hemochromatosis, idiopathic; Henoch-Schoenlein purpura; Hepatitis, chronic active and chronic progressive; Histiocytosis X; Hypereosinophilic syndrome; Idiopathic thrombocytopenic purpura; Job's syndrome; Juvenile dermatomyositis; Juvenile rheumatoid arthritis (Juvenile chronic arthritis); Kawasaki's disease; Keratitis; Keratoconjunctivitis sicca; Landry-Guillain-Barre-Strohl syndrome; Leprosy, lepromatous; Loeffler's syndrome; lupus; lupus nephritis; Lyell's syndrome; Lyme disease; Lymphomatoid granulomatosis; Mastocytosis, systemic; Mixed connective tissue disease; Mononeuritis multiplex; Muckle-Wells syndrome; Mucocutaneous lymph node syndrome; Mucocutaneous lymph node syndrome; Multicentric reticulohistiocytosis; Multiple sclerosis; Myasthenia gravis; Mycosis fimgoides; Necrotizing vasculitis, systemic; Nephrotic syndrome; Overlap syndrome; Panniculitis; Paroxysmal cold hemoglobinuria; Paroxysmal nocturnal hemoglobinuria; Pemphigoid; Pemphigus; Pemphigus erythematosus; Pemphigus foliaceus; Pemphigus vulgaris; Pigeon breeder's disease; Pneumonitis, hypersensitivity; Polyarteritis nodosa; Polymyalgia rheumatic; Polymyositis; Polyneuritis, idiopathic; Portuguese familial polyneuropathies; Pre-eclampsia/eclampsia; Primary biliary cirrhosis; Progressive systemic sclerosis (Scleroderma); Psoriasis; Psoriatic arthritis; Pulmonary alveolar proteinosis; Pulmonary fibrosis, Raynaud's phenomenon/syndrome; Reidel's thyroiditis; Reiter's syndrome, Relapsing polychrondritis; Rheumatic fever; Rheumatoid arthritis; Sarcoidosis; Scleritis; Sclerosing cholangitis; Scleroderma, Serum sickness; Sezary syndrome; Sjogren's syndrome; Stevens-Johnson syndrome; Still's disease; Subacute sclerosing panencephalitis; Sympathetic ophthalmia; Systemic lupus erythematosus; Transplant rejection; Ulcerative colitis; Undifferentiated connective tissue disease; Urticaria, chronic; Urticaria, cold; Uveitis; Vitiligo; Weber-Christian disease; Wegener's granulomatosis and Wiskott-Aldrich syndrome.

C Anti-inflammatory Therapy

In yet other embodiments, the altered polypeptides of the present invention may be used to treat inflammatory disorders that are caused, at least in part, or exacerbated by inflammation, e.g., increased blood flow, edema, activation of immune cells (e.g., proliferation, cytokine production, or enhanced phagocytosis). Exemplary inflammatory disorders include those in which inflammation or inflammatory factors (e.g., matrix metalloproteinases (MMPs), nitric oxide (NO), TNF, interleukins, plasma proteins, cellular defense systems, cytokines, lipid metabolites, proteases, toxic radicals, mitochondria, apoptosis, adhesion molecules, etc.) are involved or are present in a given area or tissue in aberrant amounts, e.g., in amounts which may be advantageous to alter, e.g., to benefit the subject. The inflammatory process is the response of living tissue to damage. The cause of inflammation may be due to physical damage, chemical substances, micro-organisms, tissue necrosis, cancer or other agents. Acute inflammation is short-lasting, lasting only a few days. If it is longer lasting however, then it may be referred to as chronic inflammation.

Inflammatory disorders include acute inflammatory disorders, chronic inflammatory disorders, and recurrent inflammatory disorders. Acute inflammatory disorders are generally of relatively short duration, and last for from about a few minutes to about one to two days, although they may last several weeks. The main characteristics of acute inflammatory disorders include increased blood flow, exudation of fluid and plasma proteins (edema) and emigration of leukocytes, such as neutrophils. Chronic inflammatory disorders, generally, are of longer duration, e.g., weeks to months to years or even longer, and are associated histologically with the presence of lymphocytes and macrophages and with proliferation of blood vessels and connective tissue. Recurrent inflammatory disorders include disorders which recur after a period of time or which have periodic episodes. Examples of recurrent inflammatory disorders include asthma and multiple sclerosis. Some disorders may fall within one or more categories.

Inflammatory disorders are generally characterized by heat, redness, swelling, pain and loss of function. Examples of causes of inflammatory disorders include, but are not limited to, microbial infections (e.g., bacterial, viral and fungal infections), physical agents (e.g., burns, radiation, and trauma), chemical agents (e.g., toxins and caustic substances), tissue necrosis and various types of immunologic reactions. Examples of inflammatory disorders include, but are not limited to, Alzheimer's; severe asthma, atherosclerosis, cachexia, CHF-ischemia, and coronary restenosis; osteoarthritis, rheumatoid arthritis, fibrosis/radiation-induced or juvenile arthritis; acute and chronic infections (bacterial, viral and fungal); acute and chronic bronchitis, sinusitis, and other respiratory infections, including the common cold; acute and chronic gastroenteritis and colitis and Crohn's diseas; acute and chronic cystitis and urethritis; acute respiratory distress syndrome; cystic fibrosis; acute and chronic dermatitis; psoriasis; acute and chronic conjunctivitis; acute and chronic serositis (pericarditis, peritonitis, synovitis, pleuritis and tendinitis); uremic pericarditis; acute and chronic cholecystis; acute and chronic vaginitis; stroke, inflammation of the brain or central nervous system caused by trauma, and ulcerative colitis; acute and chronic uveitis; drug reactions; diabetic nephropathy, and bums (thermal, chemical, and electrical). Other inflammatory disorders or conditions that can be prevented or treated with the antibodies or antigen-binding fragments of the invention include inflammation due to corneal transplantation, chronic obstructive pulmonary disease, hepatitis C, lymphoma, multiple myeloma, and osteoarthritis.

In another embodiment, the polypeptides of the invention can be used to prevent or treat neurodegenerative disorders, including, but not limited to Alzheimer's, stroke, and traumatic brain or central nervous system injuries. Additional neurodegenerative disorders include ALS/motor neuron disease, diabetic peripheral neuropathy, diabetic retinopathy, Huntington's disease, macular degeneration, and Parkinson's disease. In preferred embodiments, altered polypeptides having reduced binding affinity to FcR are used to treat nervous system disorders, as they do not cross the blood brain barrier as efficiently as those with higher FcR binding affinity. For example, in one embodiment, an altered polypeptide of the invention is injected into the spinal fluid to treat a neurodegenerative disorder.

In prophylactic applications, pharmaceutical compositions comprising a polypeptide of the invention or medicaments are administered to a subject at risk for (or having and not yet exhibiting symptoms of) a disorder treatable with a polypeptide having an Fc region, for example, an immune system disorder, in an amount sufficient to eliminate or reduce the risk, lessen the severity, or delay the outset of the disorder, including biochemical, histologic and/or behavioral symptoms of the disorder, its complications and intermediate pathological phenotypes presenting during development of the disorder.

In therapeutic applications, compositions or medicaments are administered to a subject already suffering from such a disorder in an amount sufficient to cure, or at least partially arrest, the symptoms of the disorder (biochemical, histologic and/or behavioral), including its complications and intermediate pathological phenotypes in development of the disorder. The polypeptides of the invention are particularly useful for modulating the biological activity of a cell surface antigen that resides in the blood, where the disease being treated or prevented is caused at least in part by abnormally high or low biological activity of the antigen.

In some methods, administration of agent reduces or eliminates the immune disorder, for example, inflammation. An amount adequate to accomplish therapeutic or prophylactic treatment is defined as a therapeutically- or prophylactically-effective dose. In both prophylactic and therapeutic regimes, agents are usually administered in several dosages until a sufficient immune response has been achieved.

It will be understood that the modified polypeptides of the invention can be used to treat a number of disorders not explicitly mentioned herein based on selection of the target molecule to which the polypeptide binds. It will be further recognized that any art recognized antibody or fusion protein may be modified according to the methods of the invention and used to treat a disorder for which it is indicated.

D. Methods of Administration

Altered polypeptides of the invention can be administered by startingeral, topical, intravenous, oral, intraarterial, intracranial, intraperitoneal, or intranasal means for prophylactic and/or therapeutic treatment. The term parenteral as used herein includes intravenous, intraarterial, intraperitoneal, intramuscular, subcutaneous, rectal or vaginal administration. The most typical route of administration of a protein drug is intravascular, subcutaneous, or intramuscular, although other routes can be effective. In some methods, agents are injected directly into a particular tissue where deposits have accumulated, for example intracranial injection. In some methods, antibodies are administered as a sustained release composition or device, such as a MedipadTM device. The protein drug can also be administered via the respiratory tract, e.g., using a dry powder inhalation device.

Effective doses of the compositions of the present invention, for the treatment of the above described conditions vary depending upon many different factors, including means of administration, target site, physiological state of the subject, whether the subject is human or an animal, other medications administered, and whether treatment is prophylactic or therapeutic. Usually, the subject is a human but non-human mammals including transgenic mammals can also be treated.

For passive immunization with an antibody, the dosage ranges from about 0.0001 to 100 mg/kg, and more usually 0.01 to 20 mg/kg, of the host body weight. For example dosages can be 1 mg/kg body weight or 10 mg/kg body weight or within the range of 1-10 mg/kg, preferably at least 1 mg/kg. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two polypeptides with different binding specificities are administered simultaneously, in which case the dosage of each polypeptide administered falls within the ranges indicated.

Polypeptides are usually administered on multiple occasions. Intervals between single dosages can be weekly, monthly or yearly. In some methods, dosage is adjusted to achieve a plasma antibody concentration of 1-1000 μg/ml and in some methods 25-300 μg/ml. Alternatively, polypeptides can be administered as a sustained release formulation, in which case less frequent administration is required. Dosage and frequency vary depending on the half-life of the polypeptide in the subject. In general, human antibodies show the longest half-life, followed by humanized antibodies, chimeric antibodies, and nonhuman antibodies. As discussed herein, the half-life may also depends upon the particular mutation(s) present in the altered polypeptide.

The dosage and frequency of administration can vary depending on whether the treatment is prophylactic or therapeutic. In prophylactic applications, compositions containing the present antibodies or a cocktail thereof are administered to a subject not already in the disease state to enhance the subject's resistance. Such an amount is defined to be a “prophylactic effective dose.” In this use, the precise amounts again depend upon the subject's state of health and general immunity, but generally range from 0.1 to 25 mg per dose, especially 0.5 to 2.5 mg per dose. A relatively low dosage is administered at relatively infrequent intervals over a long period of time. Some subjects continue to receive treatment for the rest of their lives.

In therapeutic applications, a relatively high dosage (e.g., from about 1 to 200 mg of antibody per dose, with dosages of from 5 to 25 mg being more commonly used) at relatively short intervals is sometimes required until progression of the disease is reduced or terminated, and preferably until the subject shows partial or complete amelioration of symptoms of disease. Thereafter, the patent can be administered a prophylactic regime.

Doses for nucleic acids encoding antibodies range from about 10 ng to 1 g, 100 ng to 100 mg, 1 μg to 10 mg, or 30-300 μg DNA per subject. Doses for infectious viral vectors vary from 10-100, or more, virions per dose.

One skilled in the art would be able, by routine experimentation, to determine what an effective, non-toxic amount of altered polypeptide would be for the purpose of treating a disorder. For example, a therapeutically active amount of a modified polypeptide may vary according to factors such as the disease stage (e.g., stage I versus stage IV tumor), age, sex, medical complications (e.g., immunosuppressed conditions or diseases) and weight of the subject, and the ability of the modified polypeptide to elicit a desired response in the subject. The dosage regimen may be adjusted to provide the optimum therapeutic response. For example, several divided doses may be administered daily, or the dose may be proportionally reduced as indicated by the exigencies of the therapeutic situation.

E. Monitoring of Treatment

Treatment of a subject suffering from a disease or disorder can be monitored using standard methods. Some methods entail determining a baseline value, for example, of an antibody level or profile in a subject, before administering a dosage of agent, and comparing this with a value for the profile or level after treatment. A significant increase (i.e., greater than the typical margin of experimental error in repeat measurements of the same sample, expressed as one standard deviation from the mean of such measurements) in value of the level or profile signals a positive treatment outcome (i.e., that administration of the agent has achieved a desired response). If the value for immune response does not change significantly, or decreases, a negative treatment outcome is indicated.

In other methods, a control value (i.e., a mean and standard deviation) of level or profile is determined for a control population. Typically the individuals in the control population have not received prior treatment. Measured values of the level or profile in a subject after administering a therapeutic agent are then compared with the control value. A significant increase relative to the control value (e.g., greater than one standard deviation from the mean) signals a positive or sufficient treatment outcome. A lack of significant increase or a decrease signals a negative or insufficient treatment outcome. Administration of agent is generally continued while the level is increasing relative to the control value. As before, attainment of a plateau relative to control values is an indicator that the administration of treatment can be discontinued or reduced in dosage and/or frequency.

In other methods, a control value of the level or profile (e.g., a mean and standard deviation) is determined from a control population of individuals who have undergone treatment with a therapeutic agent and whose levels or profiles have plateaued in response to treatment. Measured values of levels or profiles in a subject are compared with the control value. If the measured level in a subject is not significantly different (e.g., more than one standard deviation) from the control value, treatment can be discontinued. If the level in a subject is significantly below the control value, continued administration of agent is warranted. If the level in the subject persists below the control value, then a change in treatment may be indicated.

In other methods, a subject who is not presently receiving treatment but has undergone a previous course of treatment is monitored for polypeptide levels or profiles to determine whether a resumption of treatment is required. The measured level or profile in the subject can be compared with a value previously achieved in the subject after a previous course of treatment. A significant decrease relative to the previous measurement (i.e., greater than a typical margin of error in repeat measurements of the same sample) is an indication that treatment can be resumed. Alternatively, the value measured in a subject can be compared with a control value (mean plus standard deviation) determined in a population of subjects after undergoing a course of treatment. Alternatively, the measured value in a subject can be compared with a control value in populations of prophylactically treated subjects who remain free of symptoms of disease, or populations of therapeutically treated subjects who show amelioration of disease characteristics. In all of these cases, a significant decrease relative to the control level (i.e., more than a standard deviation) is an indicator that treatment should be resumed in a subject.

The polypeptide profile following administration typically shows an immediate peak in antibody concentration followed by an exponential decay. Without a further dosage, the decay approaches pretreatment levels within a period of days to months depending on the half-life of the antibody administered. For example the half-life of some human antibodies is of the order of 20 days.

In some methods, a baseline measurement of polypeptide to a given antigen in the subject is made before administration, a second measurement is made soon thereafter to determine the peak polypeptide level, and one or more further measurements are made at intervals to monitor decay of polypeptide levels. When the level of polypeptide has declined to baseline or a predetermined percentage of the peak less baseline (e.g., 50%, 25% or 10%), administration of a further dosage of polypeptide is administered. In some methods, peak or subsequent measured levels less background are compared with reference levels previously determined to constitute a beneficial prophylactic or therapeutic treatment regime in other subjects. If the measured polypeptide level is significantly less than a reference level (e.g., less than the mean minus one standard deviation of the reference value in population of subjects benefiting from treatment) administration of an additional dosage of polypeptide is indicated.

Additional methods include monitoring, over the course of treatment, any art-recognized physiologic symptom (e.g., physical or mental symptom) routinely relied on by researchers or physicians to diagnose or monitor disorders.

F. Combination Therapy

Altered polypeptides of the invention can optionally be administered in combination with other agents (including any agent from Section VIII supra) that are known or determined to be effective in treating the disorder or condition in need of treatment (e.g., prophylactic or therapeutic). In addition, the polypeptides of the invention can be conjugated to a moiety that adds functionality to the polyeptide, e.g., (e.g., PEG, a tag, a drug, or a label).

It will further be appreciated that the altered polypeptides of the instant invention may be used in conjunction or combination with any chemotherapeutic agent or agents (e.g. to provide a combined therapeutic regimen) that eliminates, reduces, inhibits or controls the growth of neoplastic cells in vivo. Exemplary chemotherapeutic agents that are compatible with the instant invention include alkylating agents, vinca alkaloids (e.g., vincristine and vinblastine), procarbazine, methotrexate and prednisone. The four-drug combination MOPP (mechlethamine (nitrogen mustard), vincristine (Oncovin), procarbazine and prednisone) is very effective in treating various types of lymphoma and comprises a preferred embodiment of the present invention. In MOPP-resistant patients, ABVD (e.g., adriamycin, bleomycin, vinblastine and dacarbazine), ChlVPP (chlorambucil, vinblastine, procarbazine and prednisone), CABS (lomustine, doxorubicin, bleomycin and streptozotocin), MOPP plus ABVD, MOPP plus ABV (doxorubicin, bleomycin and vinblastine) or BCVPP (carmustine, cyclophosphamide, vinblastine, procarbazine and prednisone) combinations can be used. Arnold S. Freedman and Lee M. Nadler, Malignant Lymphomas, in HARRISON'S PRINCIPLES OF INTERNAL MEDICINE 1774-1788 (Kurt J. Isselbacher et al., eds., 13^{th} ed. 1994) and V. T. DeVita et al., (1997) and the references cited therein for standard dosing and scheduling. These therapies can be used unchanged, or altered as needed for a particular patient, in combination with one or more modified polypeptides of the invention as described herein.

Additional regimens that are useful in the context of the present invention include use of single alkylating agents such as cyclophosphamide or chlorambucil, or combinations such as CVP (cyclophosphamide, vincristine and prednisone), CHOP (CVP and doxorubicin), C-MOPP (cyclophosphamide, vincristine, prednisone and procarbazine), CAP-BOP (CHOP plus procarbazine and bleomycin), m-BACOD (CHOP plus methotrexate, bleomycin and leucovorin), ProMACE-MOPP (prednisone, methotrexate, doxorubicin, cyclophosphamide, etoposide and leucovorin plus standard MOPP), ProMACE-CytaBOM (prednisone, doxorubicin, cyclophosphamide, etoposide, cytarabine, bleomycin, vincristine, methotrexate and leucovorin) and MACOP-B (methotrexate, doxorubicin, cyclophosphamide, vincristine, fixed dose prednisone, bleomycin and leucovorin). Those skilled in the art will readily be able to determine standard dosages and scheduling for each of these regimens. CHOP has also been combined with bleomycin, methotrexate, procarbazine, nitrogen mustard, cytosine arabinoside and etoposide. Other compatible chemotherapeutic agents include, but are not limited to, 2-chlorodeoxyadenosine (2-CDA), 2′-deoxycoformycin and fludarabine.

For patients with intermediate- and high-grade NHL, who fail to achieve remission or relapse, salvage therapy is used. Salvage therapies employ drugs such as cytosine arabinoside, carboplatin, cisplatin, etoposide and ifosfamide given alone or in combination. In relapsed or aggressive forms of certain neoplastic disorders the following protocols are often used: IMVP-16 (ifosfamide, methotrexate and etoposide), MIME (methyl-gag, ifosfamide, methotrexate and etoposide), DHAP (dexamethasone, high dose cytarabine and cisplatin), ESHAP (etoposide, methylpredisolone, HD cytarabine, cisplatin), CEPP(B) (cyclophosphamide, etoposide, procarbazine, prednisone and bleomycin) and CAMP (lomustine, mitoxantrone, cytarabine and prednisone) each with well known dosing rates and schedules.

The amount of chemotherapeutic agent to be used in combination with the modified polypeptides of the instant invention may vary by subject or may be administered according to what is known in the art. See for example, Bruce A Chabner et al., Antineoplastic Agents, in GOODMAN & GILMAN'S THE PHARMACOLOGICAL BASIS OF THERAPEUTICS 1233-1287 ((Joel G. Hardman et al., eds., 9^{th} ed. 1996).

While the modified polypeptides may be administered as described herein, it must be emphasized that in other embodiments modified polypeptides may be administered to otherwise healthy patients as a first line therapy. In such embodiments the modified polypeptides may be administered to patients having normal or average red marrow reserves and/or to patients that have not, and are not, undergoing. As used herein, the administration of modified polypeptides in conjunction or combination with an adjunct therapy means the sequential, simultaneous, coextensive, concurrent, concomitant or contemporaneous administration or application of the therapy and the disclosed antibodies. Those skilled in the art will appreciate that the administration or application of the various components of the combined therapeutic regimen may be timed to enhance the overall effectiveness of the treatment. For example, chemotherapeutic agents could be administered in standard, well known courses of treatment followed within a few weeks by radioimmunoconjugates of the present invention. Conversely, cytotoxin associated modified polypeptides could be administered intravenously followed by tumor localized external beam radiation. In yet other embodiments, the modified polypeptide may be administered concurrently with one or more selected chemotherapeutic agents in a single office visit. A skilled artisan (e.g. an experienced oncologist) would be readily be able to discern effective combined therapeutic regimens without undue experimentation based on the selected adjunct therapy and the teachings of the instant specification.

In this regard it will be appreciated that the combination of the modified polypeptide and the chemotherapeutic agent may be administered in any order and within any time frame that provides a therapeutic benefit to the patient. That is, the chemotherapeutic agent and modified polypeptide may be administered in any order or concurrently. In selected embodiments the modified polypeptides of the present invention will be administered to patients that have previously undergone chemotherapy. In yet other embodiments, the modified polypeptides and the chemotherapeutic treatment will be administered substantially simultaneously or concurrently. For example, the patient may be given the modified antibody while undergoing a course of chemotherapy. In preferred embodiments the modified antibody will be administered within I year of any chemotherapeutic agent or treatment. In other preferred embodiments the modified polypeptide will be administered within 10, 8, 6, 4, or 2 months of any chemotherapeutic agent or treatment. In still other preferred embodiments the modified polypeptide will be administered within 4, 3, 2 or 1 week of any chemotherapeutic agent or treatment. In yet other embodiments the modified polypeptide will be administered within 5, 4, 3, 2 or 1 days of the selected chemotherapeutic agent or treatment. It will further be appreciated that the two agents or treatments may be administered to the patient within a matter of hours or minutes (i.e. substantially simultaneously).

IX. Pharmaceutical Compositions

The therapeutic compositions of the invention include at least one of the modified Fc-containing polypeptides produced by a method described herein in a pharmaceutically acceptable carrier. A “pharmaceutically acceptable carrier” refers to at least one component of a pharmaceutical preparation that is normally used for administration of active ingredients. As such, a carrier may contain any pharmaceutical excipient used in the art and any form of vehicle for administration. The compositions may be, for example, injectable solutions, aqueous suspensions or solutions, non-aqueous suspensions or solutions, solid and liquid oral formulations, salves, gels, ointments, intradermal patches, creams, lotions, tablets, capsules, sustained release formulations, and the like. Additional excipients may include, for example, colorants, taste-masking agents, solubility aids, suspension agents, compressing agents, enteric coatings, sustained release aids, and the like.

Agents of the invention are often administered as pharmaceutical compositions comprising an active therapeutic agent, i.e., and a variety of other pharmaceutically acceptable components. See Remington's Pharmaceutical Science (15th ed., Mack Publishing Company, Easton, Pa. (1980)). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Polypeptides can be administered in the form of a depot injection or implant preparation, which can be formulated in such a manner as to permit a sustained release of the active ingredient. An exemplary composition comprises polypeptide at 5 mg/mL, formulated in aqueous buffer consisting of 50 mM L-histidine, 150 mM NaCl, adjusted to pH 6.0 with HCl. An exemplary generic formulation buffer is 20 mM sodium citrate, pH 6.0, 10% sucrose, 0.1% Tween 80.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (see Langer, Science 249:1527, 1990 and Hanes, Advanced Drug Delivery Reviews 28:97, 1997).

This invention is further illustrated by the following examples which should not be construed as limiting. The contents of all references, patents, and published patent applications cited throughout this application, as well as the figures and the sequence listing, are hereby incorporated by reference.

### EXAMPLES

### Example 1

### Identification of Target Residues that Influence FcγR Binding and Selection of Preferred Amino Acid Substitutions using Electrostatic Optimization

In an effort to identify the identify the position of target Fc residue(s) that are sub-optimal for FcγR binding, electrostatic charge optimization techniques were applied to a crystal structure of human Fc polypeptide complexed with CD16 (also known as FcγRIII) (see Radaev et al., J. Biol. Chem. 276:16469-16477, 2001; Sondermann et al., Nature 406:267-273, 2000). A crystal structure corresponding to an Fc/CD16b complex (PDB codes 1e4k and Iiis) was prepared using standard procedures for adding hydrogens with the program CHARMM (Accelrys, Inc., San Diego, Calif.). N-acetamide and N-methylamide patches were applied to the N-termini and C-termini, respectively.

The electrostatic charge optimization procedure utilized a previously described computational analysis (see Lee and Tidor, J. Chem. Phys. 106:8681-8690, 1997; Kangas and Tidor, J. Chem. Phys. 109:7522-7545, 1998, see also, U.S. Pat. No. 6,230,102).

Using a continuum electrostatics model, an electrostatic charge optimization was performed on each side chain of the amino acids in the Fc molecule that is located within 10 Å of the Fc/CD 16 interface. Side chains were built by performing a rotamer dihedral scan in CHARMM, using dihedral angle increments of 60 degrees, to determine the most desirable position for each side chain. Binding energies were then calculated for the wild type complexes using the Poisson-Boltzmann electrostatic energy and additional terms for the van der Waals energy and buried surface area. Optimization was performed with a net side chain charge of −1, 0, and +1, with the additional constraint that no atom's charge exceeded an absolute value of 0.85 electron charge units.

The following Table 1 shows the optimization results obtained for a selected set of residues in one of the chains (A) of the Fc molecule, using the X-ray crystal structure of the Fc/CD16 complex (PDB code 1e4k). The Mut (Mutation energy) column corresponds to the binding free energy difference (in kcal/mol) in going from the native residue to a completely uncharged sidechain isostere (i.e., a residue with the same shape but no charges or partial charges on the atoms). Negative numbers indicate a predicted increase of binding affinity.

TABLE 1
Electrostatic Optimization of Target Fc Alteration Sites

| Residue | Mut | Opt-1 | Opt0 | Opt1 |
|---|---|---|---|---|
| A 232 PRO | 0 | −0.46 | −0.04 | 0.44 |
| A 233 GLU | 0.37 | −0.09 | 0.37 | 0.88 |
| A 234 LEU | 0 | −0.67 | −0.25 | 0.29 |
| A 235 LEU | 0 | −0.59 | −0.16 | 0.97 |
| A 238 PRO | 0 | −2.18 | −1.01 | 0.58 |
| A 239 SER | −0.14 | −1.98 | −0.58 | 1.56 |
| A 240 VAL | 0 | −1.72 | −0.14 | 1.67 |
| A 241 PHE | 0.02 | −0.78 | −0.01 | 0.87 |
| A 262 VAL | 0 | −0.52 | −0.02 | 0.51 |
| A 263 VAL | 0 | −1.21 | −0.06 | 1.24 |
| A 264 VAL | 0 | −1.2 | −0.39 | 0.52 |
| A 265 ASP | 1.26 | −0.66 | 0.73 | 2.59 |
| A 266 VAL | 0 | −0.75 | −0.01 | 0.8 |
| A 267 SER | 0.01 | −0.66 | 0.01 | 0.84 |
| A 269 GLU | 0.41 | −0.04 | 0.41 | 0.9 |
| A 270 ASP | 0.77 | 0.07 | 0.68 | 1.39 |
| A 273 VAL | 0 | −0.47 | 0 | 0.52 |
| A 299 THR | 0.01 | −0.52 | 0.01 | 0.63 |
| A 322 LYS | −0.14 | −0.27 | −0.13 | 0.03 |
| A 323 VAL | 0 | −1.07 | −0.04 | 1.09 |
| A 324 SER | 0.02 | −0.16 | 0.02 | 0.28 |
| A 325 ASN | 0.18 | −1.21 | −0.49 | 0.59 |
| A 326 LYS | −0.01 | −1.23 | −1.1 | −0.87 |
| A 327 ALA | 0 | −1.12 | −0.41 | 1.04 |
| A 328 LEU | 0 | −6.92 | −6.06 | −4.64 |
| A 329 PRO | 0 | 2.78 | −0.61 | 2.4 |
| A 330 ALA | 0 | −0.39 | −0.39 | 0.42 |
| A 331 PRO | 0 | −0.54 | −0.21 | 0.2 |
| A 332 ILE | 0 | −4.17 | −2.86 | −1.18 |
| A 333 GLU | 0.29 | −0.03 | 0.29 | 0.64 |
| A 334 LYS | −0.83 | −1.68 | −1.04 | −0.28 |

The Opt-1 column corresponds to the binding free energy difference that can be obtained with an optimal charge distribution in the side chain and a net side chain charge of −1. The columns Opt0 and Opt1 correspond to the binding free energy differences with optimal charges, the net charge being 0 and +1, respectively.

Appropriate side chain mutations were then determined based on the potential gain in electrostatic binding energy observed in the optimization procedure. Based on these results and the visual inspection of the structure, mutations were designed that could take advantage of these binding free energy improvements. For instance, the designed mutation LEU234 to E uses the −0.67 kcal/mol predicted maximal free energy gain for a mutation to a side chain with a net charge of −1.

Based on these calculations, the FcγR binding affinity of 88 modified antibodies having a single mutation (i.e., 88 “single mutants”) was computationally determined. It was predicted that 31 of the single mutants would be electrostatically favorable relative to the wild-type antibody. The designed Fc protein mutant complexes were built in silico and calculation of the predicted free energy gain was determined using the same procedures as those used for wild-type complexes.

Because the Fc region consists of two identical protein chains, the mutations were applied to both protein chains. Selected results from these computational mutation calculations are shown in Table 2. Numbers represent the change in binding affinity from the wild-type to the mutant (negative meaning the mutant is more favorable). Energies are the average of the two models.

TABLE 2
Preferred Amino Acid Substitutions with enhanced FcγR binding affinity

| Mutation | Electrostatics | Full Energy |
|---|---|---|
| Leu234Asp | −4.2 | −4.6 |
| Ser239Asp | −3.5 | −2.1 |
| Ser239Glu | −2.7 | −4.0 |
| Phe241Gln | −1.2 | −1.1 |
| Ser298Asn | −2.9 | −5.8 |
| Leu328Asn | −1.3 | −0.6 |
| Leu328Asp | 2.0 | 2.3 |
| Leu328Gln | −1.7 | −0.9 |
| Leu328Glu | −3.4 | −2.5 |
| Ile332Asp | −5.1 | −4.3 |
| Ile332Gln | −0.8 | −1.0 |
| Ile332Glu | −3.6 | −3.1 |
| Ile332His | −2.3 | −2.4 |
| Lys334Asn | −0.9 | −0.9 |
| Lys334Asp | −0.9 | −0.9 |
| Lys334Gln | −1.0 | −1.0 |
| Lys334Glu | −1.0 | −1.0 |

### Example 2

### Identification of Target Residues that Influence FcγR Binding and election of Preferred Amino Acid Substitutions using Conformation Analysis

Analysis of the conformational differences between a free Fc molecule and an Fc molecule bound to CD 16b revealed several significant differences. The differences include a widening of the angle between domains CH₂ and CH₃ when Fc is bound to CD16b. By mutating the Fc protein to generate mutations that favor the CD16-bound conformation, the affinity of Fc for CD 16 was predicted to increase. The identification of altered polypeptides that favor a “bound” conformation were identified using several methods:

a) 3-D Visualization

Since the bound form of Fc has a widened angle between the CH₂ and CH₃ domains, a 3-D molecular visualizer was used to identify mutations that disfavor the unbound conformation by steric crowding. Two suitable amino acid positions were identified: A378 and D376.

Mutation that substituted A378 for an amino acid with a large sidechain were selected because the steric interaction with residues P247 and K248 was predicted to strongly disfavor the closed conformation. Accordingly, the following mutations were selected: A378K, A378Q, A378R, A378H, A378F, A378Y, A378W. Preferred mutations of D376 also included amino acids with large side chains, since D376 does not directly interact with any specific residues in CH₂, but is at a location where an increased size amino acid will not fit in the closed conformation. Therefore, the following mutationso of D376 were selected: D376R, D376K, D376H, D376F, D376Y, D376W.

Inspection of the closed and open conformations also suggested mutations that facilitate the opening of the conformation by removal of steric barriers to opening. Residue H435 (in CH₃) was identified as a potential barrier to the opening of the conformation because residue L251 (in CH₂) moves closer to H435 in the open conformation. Accordingly, the following mutants were predicted to favor the open conformation: H435A, H435S, H435G and L251A, L251S, L251G.

b) Sidechain Repacking

The second method uses the sidechain repacking technique to selectively favor the open conformation of the Fc protein. We define as “variable” positions those residues that are close (distance less than 10 Å) to the CH₂-CH₃ interface. Applying the sidechain repacking calculations to the open and closed conformations of Fc we identify the Fc sequence variants that will make the open (bound) form of Fc energetically more favorable compared to the closed form. The designed Fc sequence mutations of the open form that have a lower calculated intramolecular energy than the original Fc sequence will be built into the closed form, and the sequence mutations that result in higher calculated intramolecular energies for the closed form are selected as Fc variants for experimental expression and affinity testing.

In an effort to increase the binding affinity of an antibody Fc fragmnent to CD16, sidechain repacking techniques were applied to a crystal structure of the CD16b/Fc complex and to a model of the CD16a/Fc complex.

The first approach to modify the affinity of Fc to CD 16 using sidechain repacking was to define as variable residues in Fc that are close to the interface between Fc and CD16. For instance, residues of Fc that were determined to be within 10 Å of the interface include L234, G236A, S239, H268, and L328. All of these residues, or a subset of them, were allowed to mutate to any of the 20 naturally-occurring amino acids, and the sidechain repacking calculation predicted the following mutants as having the most favorable interaction energy between Fc and CD 16: L234Q, G236A, S239H, S239P, H268P, H268D, and L328T.

c) Afucosylation Mimicry

Analysis of the conformational differences between a free Fc molecule and an Fc molecule bound to CD16b also revealed differences in the orientation of the fucose residue that is part of the N-linked sugar attached to N297 and the amino acid residues which interact with the fucose. It was determined that the fucose residue is forced into an unfavorable state as the Fc binds to CD16 due to steric crowding or steric repulsion in the fucose interacting residues. The following residues in the neighborhood of the fucose were identified as residues that could cause unfavorable enthalpic and/or entropic effects upon binding: Y296, Q294, and R301. To reduce the enthalpic and/or entropic costs upon binding the following mutations were predicted: Y296A, Y296S, Y296N, Y296Q, Y296T, Y296H, Q294A, Q294S, Q294T, Q294N, R301A, R301K, R301N, R301Q, R301S, R301T.

### Example 4

### Construction of Altered Fc Polypeptides

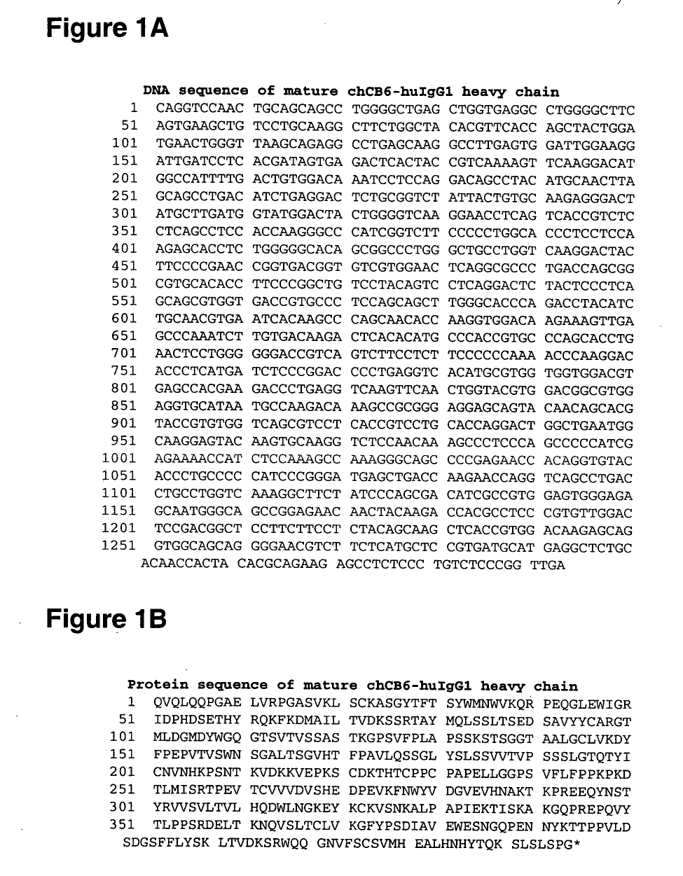

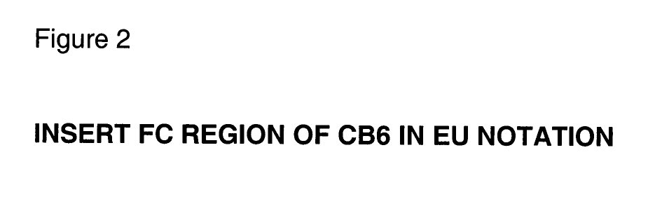

Alterations predicted by the methods of the invention were introduced into a starting polypeptide encoding the heavy chain of the murine/human chimeric IgG1 monoclonal antibody chCB6-huIgG1. FIGS. 1A and 1B display the nucleotide (SEQ ID NO. 3) and amino acid sequence (SEQ ID NO. 4) of this heavy chain respectively. The variable domain of the antibody is residues 1-120, the human IgG1 constant domain is residues 121-449. FIG. 2 displays the amino acid sequence of the Fc region of chCB6-huIgG1 in EU numbering.

CB6 is a human CD2-specific murine monoclonal antibody (IgG1, kappa) and was raised using standard techniques. Briefly, mice were immunized with CHO transfectants expressing full-length human CD2. Hybridoma supernatants were screened for binding to CD2-positive Jurkat cells. The variable domains of the CB6 heavy and light chain cDNAs were cloned by RT-PCR from total hybridoma RNA using standard molecular biological techniques. The N-terminal amino acid sequences predicted by the heavy and light chain cDNA sequences matched the N-terminal sequences of deblocked purified authentic CB6 heavy and light chains, respectively. The variable domain cDNAs were engineered and chimerized to human constant domain cDNAs using standard recombinant DNA techniques to construct chCB6-huIgG1, kappa expression vectors. The chimeric CB6 vectors were transiently co-transfected into mammalian cells and secretion of CD2-specific recombinant antibody was confirmed.

Mutations were introduced in the Fc region of the chCB6-huIgG1 heavy chain using site-directed mutagenesis by standard recombinant DNA techniques with the expression vector carrying the chCB6-huIgG1 heavy chain cDNA as template.

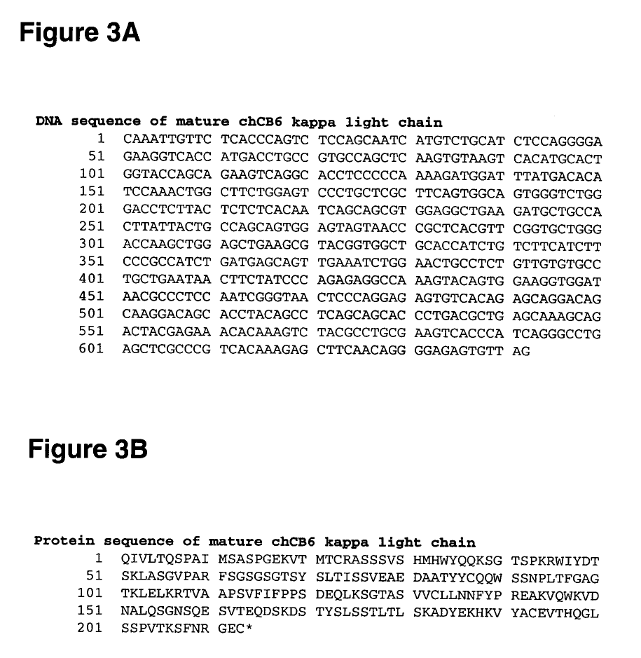

The murine CB6 light chain was carried on a separate expression vector for expression. Residues 1-106 are the murine CB6 variable domain, residues 107-213 are the human kappa constant domain. FIGS. 3A and 3B display the nucleotide (SEQ ID NO. 5) and amino acid sequence (SEQ ID NO. 6) of the light chain respectively.

Mutant antibodies were expressed by transient co-transfection of the heavy and light chain expression vectors.

### Example 5

### Assaying Effector Function of Altered Antibodies

The following example describes assays for determining the altered effector function of altered polypeptide (in particular altered antibodies) of the invention.

The variant antibodies of the invention were characterized by their ability to bind Fc gamma receptors (FcγR) and the complement molecule, C1q. In particular, the FcγR binding capabilities were measured with assays based on the ability of the antibody to form a “bridge” between the CD2 antigen and a cell bearing an Fc gamma receptor. Binding affinity for FcγRIII (CD16) was also measured in a competitive, bead-based, luminescent proximity assay. C1q binding was measured based on the ability of the antibody to form a “bridge” between the CD2 antigen and C1q. In addition, a subset of variant antibodies, were further characterized by their ability to induce antibody dependent cell-mediated cytotoxicity (ADCC).

Methods:

i) CD 16 & CD64 Bridging Assay

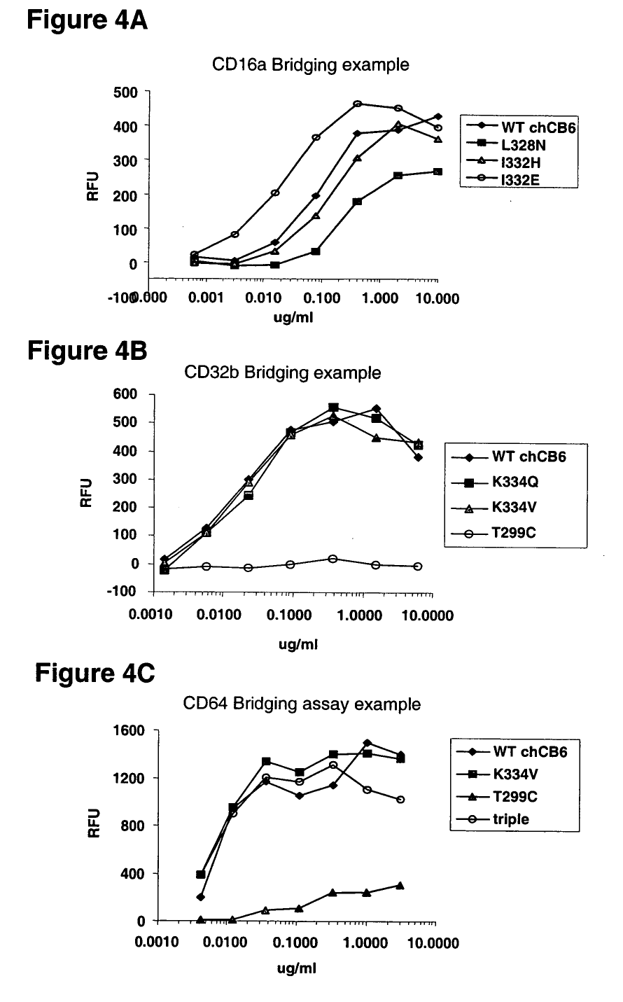

Briefly, the ability of the antibodies of the invention to bind to FcγRI (CD64) or FcγRIII (CD16) was performed using CD2 CHO-FcγR bridging assays. The ligand was produced by a monolayer of CD2-transfected Chinese Hamster Ovary (CHO) seeded into 96 well tissue culture plates (Coming Life Sciences Acton, Mass., USA) at 1×10⁵ cells/ml and grown to confluency in A-MEM with 10% dialyzed FBS, 500 nM methotrexate, L-glutamine, and penicillin /streptomycin (all tissue culture reagents from Gibco-BRL Rockville, Md., USA). The CD 16-transfected Jurkat cells were grown in RPMI with 10% FBS, 400 μg/ml Geneticin, 10 mM HEPES, sodium pyruvate, L-glutamine, and penicillin/streptomycin (Gibco-BRL) and split 1:2 one day prior to performing the assay. U937 cells, expressing FcγRI (CD64) were grown in RPMI with 10% FBS, 10 mM HEPES, sodium pyruvate, L-glutamine, and penicillin/streptomycin (Gibco-BRL), split 1:2 and activated overnight with 1000 U/ml of IFNγ one day prior to performing the assay to upregulate FcγR (CD64) expression. Titrations of the variant anti-CD2 mAbs were bound to CHO-CD2 monolayers for 30 minutes at 37° C. and the plates were washed to remove unbound mAb. The FcγR-bearing cells were labeled with 2′,7′-bis-(2-carboxyethyl)-5-(and-6)-carboxyfluorescein acetoxymethyl ester (BCECF-AM) (Molecular Probes Eugene, Oreg., USA) for 20 minutes at 37 ° C. After washing to remove excess label, 1×10⁵ of the labeled cells were incubated in the assay for 30 minutes at 37° C. Unbound FcγR cells were removed by washing several times and plates were read on a microplate reader (Cytofluor 2350 Fluorescent Microplate Reader, Millipore Corporation Bedford, Mass., USA) at an excitation wavelength of 485 nm and an emission wavelength of 530 nm. Representative competitive binding data (as expressed in relative fluorescence units) for binding of select antibody variants to CD16 and CD64 is illustrated in FIGS. 4A and 4C respectively.

ii) CD32 Bridging Assay

The ability of the antibodies of the invention to bind to FcγRII (CD32) was performed using FcγRII CHO-CD2 Jurkat bridging assays. These assays are similar to those described above but the format is inverted. Briefly, titrations of the variant anti-CD2 mAbs were bound to CHO-FcγRII monolayers for 30 minutes at 37° C. followed by the addition of fluorescently labeled CD2-bearing Jurkat cells without a wash in between steps. CHO-FcγRII cells were grown in Alpha MEM, 10% FBS, 400 μg/ml Geneticin, L-glutamine, and penicillin/streptomycin (Gibco-BRL) and seeded into 96 well plates as described above. CD2-bearing Jurkat cells were grown in RPMI with 10% FBS, 10 mM HEPES, sodium pyruvate, L-glutamine, and penicillin/streptomycin (Gibco-BRL) and split 1:2 one day prior to performing the assay. Representative competitive binding data (as expressed in relative fluorescence units) for binding of select antibody variants to CD32 is illustrated in FIG. 4B.

iii) C1q Binding Assay

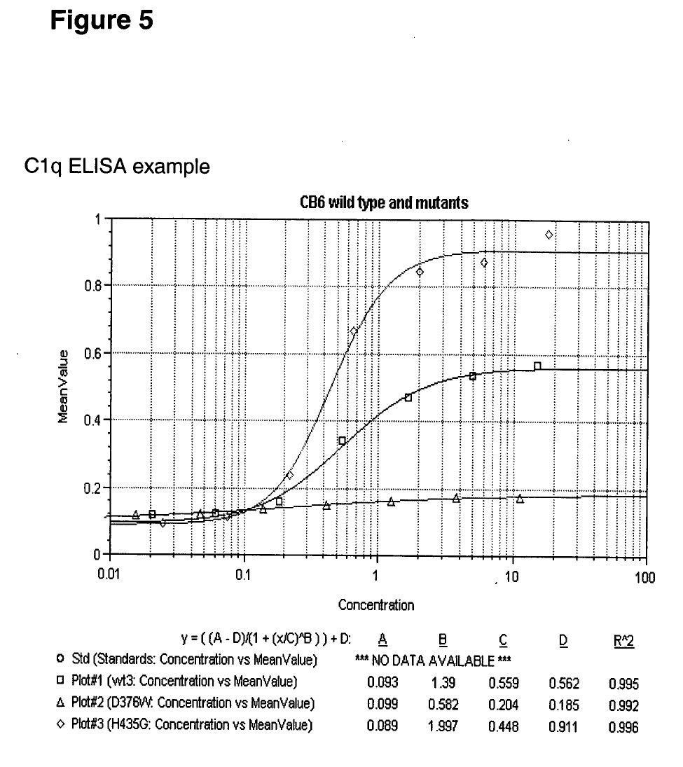

The C1q binding assay was performed by coating 96 well Maxisorb ELISA plates (Nalge-Nunc Rochester, N.Y., USA) with 50 μl recombinant soluble human CD2 at 10 μg/ml overnight at 4° C. in PBS. The wells were aspirated and washed three times with wash buffer (PBS, 0.05% Tween 20) and blocked for >1 h with 200 μl/well of block/diluent buffer (0.1 M Na₂HPO₄, pH 7, 0.1 M NaCl, 0.05% Tween 20, 0.1% gelatin). The antibody to be tested was diluted in block/diluent buffer starting at 15 μg/ml with 3-fold dilutions. 50 μl were added per well, and the plates incubated for 2 h at room temperature. After aspirating and washing as above, 50 μl/well of 2 μg/ml of Sigma human C1q (C0660) diluted in block/diluent buffer was added and incubated for 1.5 h at room temperature. After aspirating and washing as above, 50 μl/well of chicken anti human C1q (Cedarlane laboratories CL2101AP), diluted 2,000-fold in block/diluent buffer, was added. After incubation for 1.5 h at room temperature, the wells were aspirated and washed as above. 50 μl/well of donkey F(ab′)₂ anti chicken IgY HRP conjugate (Jackson ImmunoResearch 703-036-155) diluted to 1:5,000 in block/diluent was then added, and the wells incubated for 1 h at room temperature. After aspirating and washing as above, 100 μl TMB substrate (420 μM TMB, 0.004% H₂O₂ in 0.1 M sodium acetate/citric acid buffer, pH 4.9) was added and incubated for 2 min before the reaction was stopped with 100 μl 2 N sulfuric acid. The absorbance was read at 450 nm with a Softmax P instrument, and Softmax software was used to determine the relative binding affinity (C value) with a 4-parameter fit. Representative C1q binding data for select antibody variants in comparison to a wild-type antibody is illustrated in FIG. 5.

iv) AlphaScreen Binding Assay

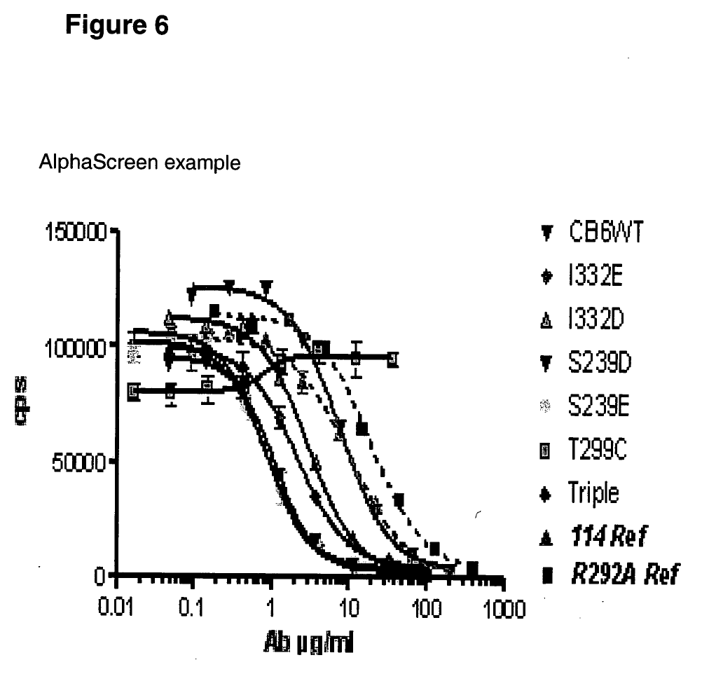

The relative binding affinities to FcγRIII (CD16), of the variant antibodies, was determined using an AlphaScreen assay (Amplified Luminescent Proximity Homogeneous Assay, PerkinElmer, MA, USA). Laser excitation of a donor bead excites oxygen, which if in close proximity to an acceptor bead generates a cascade of chemiluminescent events, ultimately leading to fluorescence emission at 520-620 nm. The AlphaScreen assay was performed in a competitive format in which GST-tagged FcγRIII in complex with a biotinylated anti-GST monoclonal antibody (Calbiochem San Diego, Calif.) was captured on streptavidin-donor beads (PerkinElmer) and a huIgG1 antibody was directly conjugated to acceptor beads (PerkinElmer). The addition of the variant antibodies competes with the FcγRIII-huIgG1 interaction resulting in reduced fluorescence. Briefly, titrations of the variant antibodies were incubated with 0.2 μg/ml of GST-FcγRIII and 0.5 μg/ml of biotinylated anti-GST mAb in 384 well white plates (Costar) at room temperature for 30 minutes followed by the addition of huIgG1-conjugated acceptor beads and streptavidin donor beads at 20 μg/ml. The reaction was carried out for one hour in a 25 μl volume and plates were read in the Fusion Alpha reader (PerkinElmer). Representative AlphaScreen binding data for binding of select antibody variants to CD16 is illustrated in FIG. 6.

v) ADCC Cytolysis Assay

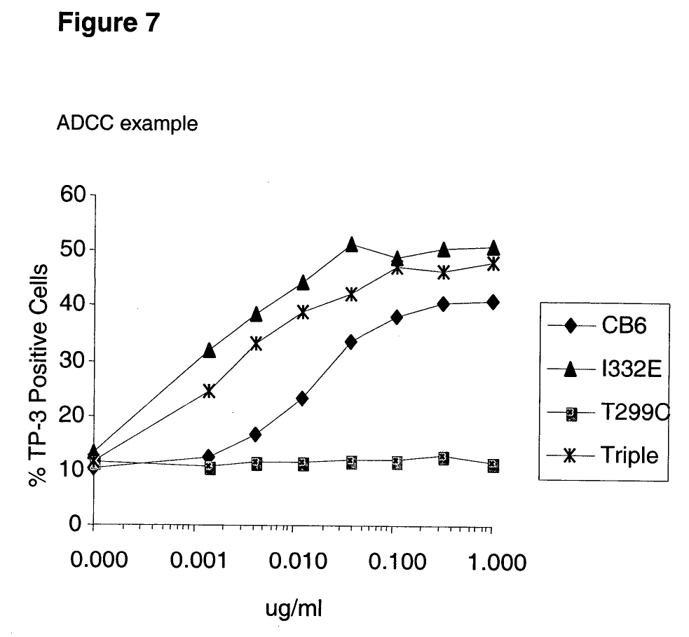

The ability of the variant antibodies to mediate ADCC was measured in a novel non-radioactive, fluorescence-based cytolysis assay utilizing autologous T lymphocytes and natural killer (NK) cells from a single donor as target cells and effector cells respectively. NK and T cells were isolated from 100 ml of whole blood using Stem Cell Technologies (Vancouver, BC, CA) Easy Sep system. The T lymphocyte target cells were labeled for one minute with 1 μM of the membrane dye PKH-26 (Sigma St Louis, Mo., USA) according to the manufacturer's instructions. The isolated NK and T cells were re-suspended in RPMI-1640 with 10% heat inactivated FBS, and 2 mM L-glutamine (Gibco-BRL) at 1×10⁶ cells/ml. Fifty microliters (5×10⁴) of labeled T cells and 50 μl (5×10⁴) of NK cells are added to 50 μl of titrated antibody solutions in a 96 well, round bottom, tissue culture plate (Corning) for a 1:1 effector-to-target ratio in a total volume of 150 μl/well. After 4 hours in culture at 37° C., 5 μl of a 0.5 μM concentration of the DNA binding dye TO-P 3 (Molecular Probes Eugene, Oreg., USA) was added to label cells with lost membrane integrity. The plate was spun at 500×g to pellet the cells and after decanting the assay buffer the cells were fixed with 100 μl/well of 2% formaldehyde in PBS. Analysis was performed using a FACScan (Becton-Dickinson Franklin Lakes, N.J., USA) fluorescence assisted cell sorter. The percentage of target cell cytolysis is determined using FlowJo software (Ashland, Oreg., USA). Live targets cells appear singly labeled with PKH-26, lysed target cells are dually labeled with PKH-26 and TO-P 3 and lysed effector cells, if present, appear as singly labeled with TO-P-3. Representative cytolysis data for select antibody variants is illustrated in FIG. 7.

Summary of Results:

Table 3 summarizes the indicated assay results for altered antigen-dependent effector functions of altered Fc-polypeptides comprising single amino acid mutations predicted using the electrostatic modeling methods described supra. Mutations that resulted in enhanced or reduced binding to the indicated Fc binding protein (ie. FcγR or complement protein), as well as enhanced or reduced ADCC activity, are indicated by upward and downward pointing arrows respectively. In addition, the proportional increase or decrease is indicated, e.g. ↓4X indicates a four-fold decrease in binding.

TABLE 3
Effector Function of Fc-polypeptides containing Single Amino
Mutations Predicted by Electrostatic Optimization

| | Bridging | Bridging | C1q | Alpha screen | ADCC | Bridging | Bridging |
|---|---|---|---|---|---|---|---|
| | CD16a (F158) | CD16a (F158) | ELISA | CD16a (V158) | NK cells | CD64 | CD32b |
| Mutation | transient | purified | purified | purified | purified | purified | purified |
| L234D | ↓ 4X | | | | | | |
| S239D | ↑ 6X | ↑ 6X | ↓ 2X | ↑ 9X | | =WT | =WT |
| S239E | ↑ 4X | ↑ 3X | ↓ 2X | ↑ 8X | | =WT | =WT |
| F241H | ↓ 9X | | | | | | |
| F241Q | ↓ 30X | | | | | | |
| D265E | ↓ (dead) | | | | | | |
| D270E | =WT | | | | | | |
| E293D | ↓ 4X | | | | | | |
| Y296F | ↓ 2X | | | | | | |
| S298N | ↓ 13X | | | | | | |
| K326D | =WT | =WT | ↑ 2X | | | =WT | =WT |
| K326E | =WT | | | | | | |
| K326N | =WT | | | | | | |
| K326Q | =WT | =WT | =WT | | | =WT | =WT |
| L328D | ↓ (dead) | ↓ (dead) | ↓ (dead) | | | =WT | =WT |
| L328E | ↓ 30X | ↓ 9X | ↓ (dead) | | | ↓ 14X | =WT |
| L328N | ↓ 2X | ↓ 10X | ↓ (dead) | | | =WT | ↓ 3X |
| L328Q | ↓ 3x | ↓ 2X | ↓ (dead) | | | =WT | =WT |
| I332D | ↑ 15X | ↑ 9X | =WT | ↑ 4X | | =WT | =WT |
| I332E | ↑ 20X | ↑ 7X | =WT | ↑ 8X | ↑ 24X | =WT | =WT |
| I332H | =WT | ↓ 2X | =WT | | | =WT | =WT |
| I332Q | ↑ 2X | ↑ 2X | =WT | | | =WT | =WT |
| E333D | =WT | =WT | ↓ 2X | | | =WT | =WT |
| K334D | =WT | =WT | =WT | | | =WT | =WT |
| K334E | =WT | =WT | =WT | | | =WT | =WT |
| K334N | =WT | =WT | =WT | | | =WT | =WT |
| K334Q | =WT | =WT | =WT | | | =WT | =WT |
| K334R | =WT | =WT | ↑ 4X | | | ↓ 5X | =WT |
| K334V | ↑ 3X | =WT | =WT | | | =WT | =WT |
| K338M | ↓ 6X | | | | | | |

Assay controls

| | | | | | | | |
|---|---|---|---|---|---|---|---|
| T299C | ↓ (dead) | ↓ (dead) | ↓ (dead) | ↓ (dead) | ↓ (dead) | ↓ (dead) | ↓ (dead) |
| Triple Mutation | ↑ 10X | ↑ 5X | ↓ 2X | ↑ 4X | ↑ 21X | ↓ 4X | =WT |
| S298A, E333A, K334A | | | | | | | |

The results demonstrate that antibodies comprising mutations at EU positions 239, 332, and 334, in particular the mutations S239D, S239E, I332D, I332E, I332Q, K334V resulted in enhanced apparent binding affinity to CD16a. In contrast, many of the altered antibodies (e.g. those containing mutations at EU positions 241, 265, 293, 296, 298, 328, and 338) exhibited a reduced apparent binding affinity CD16a. For example, mutations at EU positions 241 (F241Q), 298 (S298N), and 328 (L328D, L328E) exhibited a pronounced decrease in binding affinity for CD 16 (e.g. a more than 10-fold decrease in apparent binding affinity).

Some mutations also resulted in reduced binding affinity for other Fc gamma receptors. For example, the mutations L328E and L328N resulted in decreased binding to the CD64 amd CD32b respectively. Additionally, several mutations resulted in enhanced (e.g. K326D, K334R) or reduced (e.g. S239D, S239E) binding to the complement protein C1q.

Table 4 summarizes the indicated assay results for altered antigen-dependent effector functions of altered Fc-polypeptides comprising a combination of amino acid mutations predicted using the electrostatic modeling methods described supra. Most double mutants exhibited an increased binding to CD16a. In particular, the double mutants S239D/I332E and S239D/I332D exhibited a more than 10-fold increase in binding affinity as measured by at least one binding assay.

TABLE 4
Effector function of Fc polypeptides containing a Combination amino
acid mutations predicted by Electrostatic Optimization

| | | | | | | | |
|---|---|---|---|---|---|---|---|
| | Bridging | Bridging | C1q | Alpha screen | ADCC | Bridging | Bridging |
| | CD16a (F158) | CD16a (F158) | ELISA | CD16a (V158) | NK cells | CD64 | CD32b |
| Mutation | transient | purified | purified | purified | purified | purified | purified |

a) Double Mutants

| | | | | |
|---|---|---|---|---|
| S239E/I332D | ↑ 3X | ↑ 8X | =WT | ↑ 7X |
| S239E/I332E | ↑ 3X | ↑ 6X | =WT | ↑ 10X |
| S239D/I332D | ↑ 4X | ↑ 6X | =WT | ↑ 15X |
| S239D/I332E | ↑ 12X | ↑ 8X | =WT | ↑ 26X |
| S239D/A378F | ↑ 5X | | | |
| S239D/A378K | ↑ 5X | | | |
| S239D/A378W | ↑ 4X | | | |
| S239D/A378Y | ↑ 4X | | | |
| S239D/H435G | ↑ 3X | | | |
| S239D/H435S | ↑ 3X | ↑ 6X | | |
| I332D/A378F | ↑ 5X | | | |
| I332D/A378K | =WT | | | |
| I332D/A378W | ↑ 5X | | | |
| I332D/A378Y | ↑ 5X | | | |
| I332D/H435G | =WT | | | |
| I332D/H435S | =WT | | | |
| I332D/L261A | ↓ 2X | | | |

Table 5 summarizes the indicated assay results for altered antigen-dependent effector functions of altered Fc-polypeptides comprising amino acid mutations predicted by conformational analysis of glycan interacting residues. Most of these mutants exhibited decreased binding to CD16a or C1q. In particular, the mutants E294S, Y296A, Y296H, Y296S, and R301Q exhibited a more than 10-fold decrease in binding affinity to one or both Fc-binding proteins as measured by at least one binding assay.

TABLE 5
Effector function of Fc polypeptides containing mutations predicted by
Conformational Analysis of Glycan interacting residues

| | Bridging | C1q | Bridging | C1q |
|---|---|---|---|---|
| | CD16a (F158) | ELISA | CD16a (F158) | ELISA |
| Mutation | transient | transient | purified | purified |
| E294A | =WT | =WT | =WT | ↓3X |
| E294N | ↓8X | ↓7X | | |
| E294S | ↓12X | =WT | | |
| E294T | ↓8X | =WT | | |
| Y296A | ↓13X | ↓5X | ↓5X | ↓3X |
| Y296H | ↓18X | ↓3X | | |
| Y296Q | ↓5X | ↓4X | | |
| Y296S | ↓(dead) | ↓4X | ↓20X | ↓5X |
| Y296T | ↓(dead) | ↓3X | | |
| R301A | ↓5X | =WT | | |
| R301K | ↓5X | =WT | | |
| R301N | ↓5X | ↓3X | | |
| R301Q | ↓13X | =WT | | |
| R301S | =WT | =WT | =WT | |
| R301T | =WT | =WT | | |

Table 6 summarizes the indicated assay results for altered antigen-dependent effector functions of altered Fc-polypeptides comprising amino acid mutations predicted by 3-D visualization of CD16 binding. The results demonstrate that many mutants resulted in reduced binding to CD16a. In particular, the mutants L251, D376K, and D376W, exhibited a more than 10-fold decrease in binding affinity to CD 16 as measured by at least one binding assay. In contrast, many of the mutants resulted in enhanced binding to the complement protein C1q. For example, the mutants A378F, A378W, and A378Y exhibited a more than 5-fold increase in binding affinity to C1q.

TABLE 6
Effector function of Fc polypeptides containing mutations predicted by
3-D Visualization

| | Bridging | C1q | Bridging | C1q |
|---|---|---|---|---|
| | CD16a (F158) | ELISA | CD16a (F158) | ELISA |
| Mutation | transient | transient | purified | purified |
| L251A | =WT | ↑3X | | ↑2X |
| L251G | ↓(dead) | ↑3X | | |
| L251S | =WT | =WT | | |
| D376H | ↓6X | =WT | | |
| D376K | ↓15X | =WT | | |
| D376R | ↓9X | =WT | | |
| D376W | ↓15X | ↓(dead) | ↓3X | ↓(dead) |
| D376Y | =WT | =WT | ↓3X | ↓3X |
| A378F | =WT | ↑6X | * | |
| A378H | ↓6X | =WT | | |
| A378K | =WT | ↑4X | * | |
| A378Q | =WT | =WT | | |
| A378R | =WT | =WT | | |
| A378W | =WT | ↑7X | * | |
| A378Y | =WT | ↑6X | * | |
| H435A | ↓4X | =WT | | |
| H435G | ↓4X | ↑4X | * | |
| H435S | ↓5x | ↑4X | | |

*purified with poor yields

Table 7 summarizes the indicated assay results for altered antigen-dependent effector functions of altered Fc-polypeptides comprising amino acid mutations predicted by optimization of side chain repacking. The results demonstrate that many mutants resulted in reduced binding to CD16a. In particular, the mutants S239H and S239P exhibited complete abrogation of binding to CD16a. In contrast, the mutant H268D exhibited increased binding to CDl6a.

TABLE 7
Effector function of Fc polypeptides containing mutations predicted by
optimization of side chain repacking

| | Bridging |
|---|---|
| | CD16a |
| | (F158) |
| Mutation | transient |
| L234Q | ↓5X |
| G236A | ↓2X |
| S239H | ↓(dead) |
| S239P | ↓(dead) |
| H268P | ↓3X |
| H268D | ↑3X |
| L328T | ↓3X |
| A330H | =WT |

### EOUIVALENTS

Those skilled in the art will recognize, or be able to ascertain using no more than routine experimentation, many equivalents to the specific embodiments of the invention described herein. Such equivalents are intended to be encompassed by the following claims.

1. An altered polypeptide comprising at least an FcγR binding portion of an Fc region wherein the polypeptide comprises at least one mutation compared to a starting polypeptide and wherein the at least one mutation is selected from the group consisting of:
a substitution at EU amino acid position 236;
a substitution at EU amino acid position 239 with proline;
a substitution at EU amino acid position 241 with glutamine or histidine;
a substitution at EU amino acid position 251 with a non-polar amino acid or serine;
a substitution at EU amino acid position 265 with a negatively charged amino acid;
a substitution at EU amino acid position 268 with proline or a negatively charged amino acid;
a substitution at EU amino acid position 294 with serine, threonine, or asparagine;
a substitution at EU amino acid position 301 with serine, threonine, asparagine, glutamine or a charged amino acid;
a substitution at EU amino acid position 328 with lysine;
a substitution at EU amino acid position 332 with lysine;
a substitution at EU amino acid position 376 with a polar amino acid or a charged amino acid;
a substitution at EU amino acid position 378 with a charged amino acid, phenylalanine, glutamine, arginine, tyrosine, or tryptophan;
a substitution at EU amino acid position 388; and
a substitution at EU amino acid position 435 with a polar amino acid or glycine.

2. An altered polypeptide comprising at least an FcγR binding portion of an Fc region wherein the polypeptide comprises at least one mutation compared to a starting polypeptide and wherein the at least one mutation is selected from the group consisting of:
a substitution of glycine at EU amino acid position 236;
a substitution of serine at EU amino acid position 239 with proline;
a substitution of phenylalanine at EU amino acid position 241 with glutamine or histidine;
a substitution of leucine at EU amino acid position 251 with a non-polar amino acid or serine;
a substitution of aspartate at EU amino acid position 265 with a negatively charged amino acid;
a substitution of histidine at EU amino acid position 268 with proline or a negatively charged amino acid;
a substitution of glutamine or glutamate at EU amino acid position 294 with serine, threonine, or asparagine;
a substitution of arginine at EU amino acid position 301 with serine, threonine, asparagine, glutamine or a charged amino acid;
a substitution of leucine at EU amino acid position 328 with lysine;
a substitution of isoleucine at EU amino acid position 332 with lysine;
a substitution of asparagine at EU amino acid position 376 with a polar amino acid or a charged amino acid;
a substitution of alanine at EU amino acid position 378 with a charged amino acid, phenylalanine, glutamine, arginine, tyrosine, or tryptophan;
a substitution of glutamate at EU amino acid position 388; and
a substitution of histidine at EU amino acid position 435 with a polar amino acid or glycine.

3. The altered polypeptide of claim 1, wherein the amino acid at any of EU amino acid positions 236 or 388 is replaced with a non-polar amino acid, a charged amino acid, or a polar amino acid.

4. The altered polypeptide of claim 3, wherein the charged amino acid is a negatively charged amino acid.

5. The altered polypeptide of claim 4, wherein the negatively charged amino acid is selected from the group consisting of aspartate and glutamate.

6. The altered polypeptide of claim 3, wherein the charged amino acid is a positively charged amino acid.

7. The altered polypeptide of claim 6, wherein the positively charged amino acid is selected from the group consisting of arginine, histidine, and lysine.

8. The altered polypeptide of claim 3, wherein the polar amino acid is selected from the group consisting of methionine, phenylalanine, tryptophan, serine, tyrosine, asparagine, glutamine, and cysteine.

9. The altered polypeptide of claim 3, wherein the non-polar amino acid is selected from the group consisting of alanine, leucine, isoleucine, valine, glycine, and proline.

10. The altered polypeptide of claim 1, further comprising a mutation selected from the group consisting of:
a substitution at EU amino acid position 234 with aspartate or glutamine;
a substitution at EU amino acid position 239 with aspartate, glutamate, or histidine;
a substitution at EU amino acid position 270 with glutamate;
a substitution at EU amino acid position 292 with alanine;
a substitution at EU amino acid position 293 with aspartate;
a substitution at EU amino acid position 294 with alanine or asparagine;
a substitution at EU amino acid position 296 with alanine, serine, asparagine, glutamine, threonine, histidine, or phenylalanine;
a substitution at EU amino acid position 298 with alanine or asparagine;
a substitution at EU amino acid position 301 with alanine;
a substitution at EU amino acid position 326 with aspartate, glutamate, asparagine, or glutamine;
a substitution at EU amino acid position 328 with asparagine, aspartate, glutamate, glutamine, or threonine;
a substitution at EU amino acid position 330 with histidine or leucine;
a substitution at EU amino acid position 332 with aspartate, glutamate, glutamine, or histidine;
a substitution at EU amino acid position 333 with aspartate;
a substitution at EU amino acid position 334 with asparagine, aspartate, glutamine, glutamate, valine, or arginine; and
a substitution at EU amino acid position 338 with methionine.

11. An altered polypeptide comprising at least an FcγR binding portion of an Fc region wherein the polypeptide comprises at least two mutations compared to a starting polypeptide and wherein the at least two mutations are selected from the group consisting of:
a substitution at EU position 239 with glutamate or asparate and a substitution of EU position 378 with phenylalanine, tryptophan, tyrosine, glycine, or serine;
a substitution at EU position 332 with aspartate and a substitution of EU position 378 with phenylalanine, lysine, tryptophan, or tyrosine;
a substitution at EU position 332 with aspartate and a substitution of EU position 435 with glycine or serine; and
a substitution at EU position 332 with aspartate and a substitution of EU position 261 with alanine.

12. The altered polypeptide of claim 1, wherein the altered polypeptide is an antibody or fragment thereof.

13. The altered polypeptide of claim 1, wherein the altered polypeptide is a fusion protein.

14. The altered polypeptide of claim 1, wherein the FcγR binding portion or the Fc region is derived from a human antibody.

15. The altered polypeptide of claim 14, wherein the FcγR binding portion comprises a complete Fc region.

16. The altered polypeptide of claim 15, wherein the starting polypeptide comprises the amino acid sequence of SEQ ID NO. 2.

17. The altered polypeptide of claim 12, wherein the antibody is of the IgG isotype.

18. The altered polypeptide of claim 17, wherein the IgG isotype is of the IgG1 subclass.

19. The altered polypeptide of claim 12 wherein the polypeptide comprises one or more non-human amino acids residues in a complementarity determining region (CDR) of V_{L} or V_{H}.

20. The altered polypeptide of claim 12, wherein the polypeptide binds (a) an antigen and (b) an FcR.

21. The altered polypeptide of claim 20, wherein the antigen is a tumor-associated antigen.

22. The altered polypeptide of claim 12, wherein the polypeptide binds (a) a ligand and (b) an FcR.

23. The altered polypeptide of claim 20, wherein the FcR is an FcγR.

24. The altered polypeptide of claim 20, wherein the polypeptide binds the FcR with different binding affinity than the starting polypeptide that does not contain the mutation.

25. The altered polypeptide of claim 24, wherein the binding affinity of the altered polypeptide is about 1.5-fold to about 100-fold greater.

26. The altered polypeptide of claim 24, wherein the binding affinity of the altered polypeptide is about 1.5-fold to about 100-fold lower.

27. The altered polypeptide of claim 12 wherein the altered polypeptide, when administered to a patient, exhibits an antigen-dependent effector function that is different from the starting polypeptide that does not contain the mutation.

28. The altered polypeptide of claim 1, wherein the altered polypeptide binds to Protein A or G.

29. A pharmaceutical composition comprising the altered polypeptide of claim 1.

30. A nucleic acid molecule comprising a sequence encoding the polypeptide of claim 1.

31. The nucleic acid molecule of claim 30, which is in an expression vector.

32. A host cell comprising the expression vector of claim 31.

33. A method for treating a patient suffering from a disorder, the method comprising administering to the patient an altered polypeptide comprising at least an FcγR binding portion of an Fc region which comprises at least one mutation selected from the group consisting of:
a substitution of leucine at EU amino acid position 251 with alanine or glycine;
a substitution of histidine at EU amino acid position 268 with aspartate;
a substitution of alanine at EU amino acid position 330 with leucine or histidine;
a substitution of isoleucine at EU amino acid position 332 with aspartate, glutamate, or glutamine;
a substitution of lysine at EU amino acid position 334 with arginine;
a substitution of alanine at EU amino acid position 378 with phenylalanine, lysine, tryptophan, or tyrosine; and
a substitution of histidine at EU amino acid position 435 with glycine or serine wherein the altered polypeptide exhibits an antigen-dependent effector function that is enhanced relative to the starting polypeptide that does not contain the mutation.

34. The method of claim 33 wherein the altered polypeptide further comprises of a serine at EU amino acid position 239 with aspartate or glutamate.

35. The method of claim 34, wherein the altered polypeptide comprises two mutations, wherein the two mutations are selected from the group consisting of: S239E/I332D, S239E/I332E, S239D/I332D, S239D/I332E, S239D/A378F, S239D/A378K, S239D/A378F, S239D/A378W, S239D/A378Y, S239D/A378G, S239D/A378S, I332D/A378F, I332D/A378W, or I332D/A378Y.

36. A method for treating a patient suffering from a disorder, the method comprising administering to the patient an an altered polypeptide comprising at least an FcγR binding portion of an Fc region which comprises at least one mutation selected from the group consisting of:
a substitution of glycine at EU amino acid position 236 with alanine;
a substitution of serine at EU amino acid position 239 with proline;
a substitution of phenylalanine at EU amino acid position 241 with glutamine or histidine;
a substitution of leucine at EU amino acid position 251 with glycine;
a substitution of leucine at EU amino acid position 261 with alanine;
a substitution of aspartate at EU amino acid position 265 with glutamate;
a substitution of leucine at EU amino acid position 268 with proline;
a substitution of glutamate at EU amino acid position 293 with aspartate;
a substitution of glutamate at EU amino acid position 294 with serine or threonine;
a substitution of arginine at EU amino acid position 301 with lysine, asparagine, glutamine, serine, or threonine;
a substitution of leucine at EU amino acid position 328 with glutamine, aspartate, lysine, or threonine;
a substitution of isoleucine at EU amino acid position 332 with lysine;
a substitution of asparagine at EU amino acid position 376 with arginine, lysine, histidine, phenylalanine, or tryptophan;
a substitution of alanine at EU amino acid position 378 with histidine; and
a substitution of histidine at EU amino acid position 435 with alanine, serine, or glycine wherein the altered polypeptide exhibits an antigen-dependent effector function that is reduced relative to the starting polypeptide that does not contain the mutation.

37. A method of producing the altered polypeptide of claim 1, the method comprising:
(a) transfecting a cell with the nucleic acid molecule comprising a nucleotide sequence that encodes the altered polypeptide; and
(b) purifying the altered polypeptide from the cell or cell supernatant.

38. A method of producing the antibody of claim 1, the method comprising:
(a) providing a first nucleic acid molecule comprising a nucleotide sequence that encodes the variable (V_{L}) and constant regions (C_{L}) of the antibody's light chain;
(b) providing a second nucleic acid molecule comprising a nucleotide sequence that encodes the variable (V_{H}) and constant regions (CH₁, CH₂, and CH₃) of the antibody's heavy chain;
(c) transfecting a cell with the first and second nucleic acid molecules under conditions that permit expression of the altered antibody comprising the encoded light and heavy chains; and
(d) purifying the antibody from the cell or cell supernatant.

39. The method of claim 38, wherein the cell is a 293 cell.

40. A method for identifying a polypeptide with an altered binding affinity for a FcγR compared to a starting polypeptide, the method comprising:
(a) determining a spatial representation of an optimal charge distribution of the amino acids of the starting polypeptide and an associated change in binding free energy of the starting polypeptide when bound to the FcγR in a solvent;
(b) identifying at least one candidate amino acid residue position of the starting polypeptide to be modified to alter the binding free energy of the starting polypeptide when bound to the FcγR; and
(c) identifying an elected amino acid at the amino acid position, such that substitution of the elected amino acid into the starting polypeptide results in an altered polypeptide with an altered binding affinity for the FcγR.

41. The method of claim 40, further comprising incorporating the elected amino acid in the starting polypeptide to form an altered polypeptide.

42. The method of claim 41, further comprising calculating the change in the free energy of binding of the altered Fc-containing polypeptide when bound to the FcγR, as compared to the starting polypeptide when bound to the FcγR.

43. The method of claim 42, wherein the calculating step first comprises modeling the mutation in the starting polypeptide in silico, and then calculating the change in free energy of binding.

44. The method of claim 43, wherein the calculating step uses at least one determination selected from the group consisting of a determination of the electrostatic binding energy using a method based on the Poisson-Boltzmann equation, a determination of the van der Waals binding energy, and a determination of the binding energy using a method based on solvent accessible surface area.

45. The method of claim 43, wherein the amino acid substitution results in incorporation of an elected amino acid with a different charge than the candidate amino acid.

46. The method of claim 43, wherein the amino acid substitution results in incorporation of an elected amino acid with a different solvation effect than the candidate amino acid.

47. The method of claim 43, wherein the amino acid substitution results in incorporation of an elected amino acid with a different dielectric constant than the candidate amino acid.

48. The method of claim 43, wherein the substitution increases the free energy of binding between altered Fc-containing polypeptide and FcγR when bound in a solvent, thereby decreasing binding affinity of the altered Fc-containing polypeptide for FcγR.

49. The method of claim 43, wherein the substitution decreases the free energy of binding between altered Fc-containing polypeptide and FcγR when bound in a solvent, thereby increasing binding affinity of the altered Fc-containing polypeptide for FcγR.

50. An altered polypeptide comprising at least one amino acid mutation not found in a starting polypeptide, wherein the altered polypeptide exhibits a different binding affinity for an FcR as compared to the starting polypeptide, and wherein the altered polypeptide comprises an amino acid sequence predicted by the method of claim 40.

51. A pharmaceutical composition comprising the polypeptide of claim 50.

52. A nucleic acid molecule comprising a nucleotide sequence encoding the polypeptide of claim 51.

53. The method of claim 52, wherein the polypeptide exhibits at least one altered antigen dependent effector function selected from the group consisting of: opsonization, phagocytosis, complement dependent cytotoxicity, antigen-dependent cellular cytotoxicity (ADCC), or effector cell modulation.

54. The method of claim 40, wherein the FcγR is an activating FcγR.

55. The method of claim 54, wherein the activating FcγR is an FcγRI, FcγRIIa, or FcγRIIIa.

56. The method of claim 54, wherein the FcγR is an inhibitory FcγR.

57. The method of claim 56, wherein the inhibitory FcγR is FcγRIIb.


SEQUENCE LISTING (10 sequences)

SEQ ID NO: 1 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 2 - protein, 106 aa, Homo sapiens [sequence omitted]

SEQ ID NO: 3 - DNA, 1344 bp, Artificial Sequence
  Description of Artificial Sequence Synthetic murine/human chimeric heavy chain sequence
caggtccaactgcagcagcctggggctgagctggtgaggcctggggcttcagtgaagctgtcctgcaaggcttctggctacacgttcaccagctactggatgaactgggttaagcagaggcctgagcaaggccttgagtggattggaaggattgatcctcacgatagtgagactcactaccgtcaaaagttcaaggacatggccattttgactgtggacaaatcctccaggacagcctacatgcaacttagcagcctgacatctgaggactctgcggtctattactgtgcaagagggactatgcttgatggtatggactactggggtcaaggaacctcagtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 4 - protein, 447 aa, Artificial Sequence
  Description of Artificial Sequence Synthetic murine/human chimeric heavy chain protein sequence
QVQLQQPGAELVRPGASVKLSCKASGYTFTSYWMNWVKQRPEQGLEWIGRIDPHDSETHYRQKFKDMAILTVDKSSRTAYMQLSSLTSEDSAVYYCARGTMLDGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - DNA, 642 bp, Artificial Sequence
  Description of Artificial Sequence Synthetic murine/human chimeric light chain sequence
caaattgttctcacccagtctccagcaatcatgtctgcatctccaggggagaaggtcaccatgacctgccgtgccagctcaagtgtaagtcacatgcactggtaccagcagaagtcaggcacctcccccaaaagatggatttatgacacatccaaactggcttctggagtccctgctcgcttcagtggcagtgggtctgggacctcttactctctcacaatcagcagcgtggaggctgaagatgctgccacttattactgccagcagtggagtagtaacccgctcacgttcggtgctgggaccaagctggagctgaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 6 - protein, 213 aa, Artificial Sequence
  Description of Artificial Sequence Synthetic murine/human chimeric light chain protein sequence
QIVLTQSPAIMSASPGEKVTMTCRASSSVSHMHWYQQKSGTSPKRWIYDTSKLASGVPARFSGSGSGTSYSLTISSVEAEDAATYYCQQWSSNPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 15 aa, Artificial Sequence
  Description of Artificial Sequence Synthetic linker peptide
GGGGSGGGGSGGGGS

SEQ ID NO: 8 - protein, 11 aa, Artificial Sequence
  Description of Artificial Sequence Synthetic linker peptide
GGGSSGGGSGG

SEQ ID NO: 9 - protein, 25 aa, Artificial Sequence
  Description of Artificial Sequence Synthetic linker peptide
GGGGSGGGGSGGGGSGGGGSGGGAS

SEQ ID NO: 10 - protein, 6 aa, Artificial Sequence
  Description of Artificial Sequence Synthetic 6xHis tag
HHHHHH